(19) United States
(12) Patent Application Publication
(10) Pub. No.: US 2023/0126893 A1
(43) Pub. Date: Apr. 27, 2023

(54) [(1,4,5-TRISUBSTITUTED-1H-PYRAZOL-3-YL)SULFANYL]ACETIC ACID DERIVATIVES, SALTS THEREOF, AND USE THEREOF AS ACTIVE HERBICIDAL INGREDIENTS

(71) Applicant: BAYER AKTIENGESELLSCHAFT, Leverkusen (DE)

(72) Inventors: Thomas MUELLER, Frankfurt (DE); Michael Gerhard HOFFMANN, Konstanz (DE); Estella BUSCATO ARSEQUELL, Frankfurt am Main (DE); Harald JAKOBI, Leverkusen (DE); Dirk SCHMUTZLER, Hattersheim (DE); Christopher Hugh ROSINGER, Hofheim am Taunus (DE); Anu Bheemaiah MACHETTIRA, Frankfurt (DE); Elisabeth ASMUS, Hösbach (DE)

(21) Appl. No.: 17/795,676

(22) PCT Filed: Jan. 28, 2021
(86) PCT No.: PCT/EP2021/051920

§ 371 Date: Jul. 27, 2022

(30) Foreign Application Priority Data
Jan. 31, 2020 (EP) 20154965.6

(51) Int. Cl. C07D 231/18; A01N 43/56; C07D 401/04; C07D 409/04; C07D 417/04; A01N 43/78; C07D 403/04; A01N 43/60

(52) U.S. Cl. CPC C07D 231/18; A01N 43/56; C07D 401/04; C07D 409/04; C07D 417/04; A01N 43/78; C07D 403/04; A01N 43/60

(57) ABSTRACT

The present invention relates to the technical field of crop protection products, especially that of herbicides for control of broad-leaved weeds and weed grasses in crops of useful plants.

Specifically, this invention relates to novel substituted [(1,4,5-trisubstituted 1H-pyrazol yl)sulfanyl]acetic acid derivatives of the formula (I) or an agrochemically acceptable salt thereof, to processes for preparation thereof and to the use thereof as herbicides.

The present invention relates to the technical field of crop protection products, especially that of herbicides for control of broad-leaved weeds and weed grasses in crops of useful plants.

Specifically, this invention relates to novel substituted [(1,4,5-trisubstituted 1H-pyrazol-3-yl)sulfanyl]acetic acid derivatives of the formula (I) or an agrochemically acceptable salt thereof, to processes for preparation thereof and to the use thereof as herbicides.

Crop protection products known to date for control of harmful plants in crops of useful plants or active ingredients for control of unwanted vegetation sometimes have disadvantages on application, be it (a) that they have no or else insufficient herbicidal activity against particular harmful plants, (b) that the spectrum of harmful plants which can be controlled with an active ingredient is not wide enough, (c) that their selectivity in crops of useful plants is too low and/or (d) that they have a toxicologically unfavourable profile. Furthermore, some active ingredients which can be used as plant growth regulators for a number of useful plants cause undesirably reduced harvest yields in other useful plants or are compatible with the crop plant only within a narrow application rate range, if at all. Some of the known active ingredients cannot be produced economically on an industrial scale owing to precursors and reagents which are difficult to obtain, or they have only insufficient chemical stabilities. In the case of other active ingredients, the activity is too highly dependent on environmental conditions, such as weather and soil conditions.

The herbicidal activity of these known compounds, in particular at low application rates, and/or their compatibility with crop plants are still in need of improvement.

EP1122244 describes 2- ({4- [5-(4-tert-butyl-3-methyl-2,6-dioxo-3,6-dihydropyrimidin-1(2H)-yl)-2-chloro-4-fluorophenoxy]-1,5-dimethyl-1H-pyrazol-3- yl)sulfanyl)propionic acid derivatives as herbicides. WO2018/080859 describes [(1,5-disubstituted 1H-pyrazol-3-yl)sulfanyl]acetic acid derivatives that have fungicidal properties. Russian Chemical Bulletin 2010, 59(9), 1786-1790 and Yakugaku Zasshi 1971, 91(3), 311-323 disclose the synthesis of [(1,5-dimethyl-4-nitro-1H-pyrazol-3-yl)sulfanyl]acetic acid derivatives and 1,2,3,4-tetrasubstituted 3-pyrazoline-5-thione derivatives. SciFinder® mentions ethyl {[4-nitro-1-phenyl-5-(1H-tetrazol-5-yl)-1H-pyrazol-3-yl]sulfanyl}acetate CAS [1360702-86-8], ethyl {[5-(dichloromethyl)-4-nitro-1-phenyl-1H-pyrazol-3-yl]sulfanyl}acetate CAS [1360691-01-5], methyl {[5-(dichloromethyl)-4-nitro-1-phenyl-1H-pyrazol-3-yl]sulfanyl}acetate CAS [1360690-82-9] and methyl {[5-(5-methyl-1,3,4-oxadiazol-2-yl)-4-nitro-1-phenyl-1H-pyrazol yl]sulfanyl}acetate CAS [1360690-60-3], without giving a literature reference.

By contrast, there has been no description to date of the use of [(1,4,5-trisubstituted 1H-pyrazol yl)sulfanyl]acetic acid derivatives or salts thereof as active herbicidal ingredients that bear one of the phenyl or hetaryl radicals having defined substitution on the pyrazole. It has now been found that, surprisingly, substituted [(1,4,5-trisubstituted 1H-pyrazol-3-yl)sulfanyl]acetic acid derivatives or salts thereof as active herbicidal ingredients that bear one of the phenyl or hetaryl radicals having defined substitution on the pyrazole are of particularly good suitability as herbicides.

The present invention thus provides substituted [(1,4,5-trisubstituted 1H-pyrazol-3-yl)sulfanyl]acetic acid derivatives of the formula (I) or an agrochemically acceptable salt thereof,

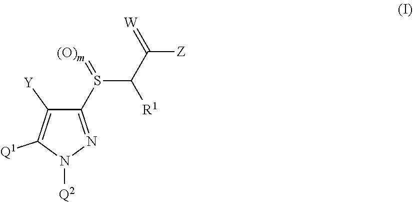

in which

Q¹ is phenyl and hetaryl,

where the phenyl and the hetaryl are unsubstituted or each independently substituted by m radicals selected from the group consisting of hydrogen, halogen, cyano, isocyano, nitro, hydroxy, (C₁-C₆)-alkyl, (C₃-C₆)-haloalkyl, (C₃-C₆)-cycloalkyl, (C₃-C₆)-halocycloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, (C₁-C₄)-haloalkyl-S(O)ₙ, CHO, (C₁-C₄)-alkyloxycarbonyl and NH₂ (amino),

Q² is phenyl,

which is unsubstituted or in each case independently substituted by m radicals selected from the group consisting of hydrogen, halogen, cyano, isocyano, NO₂, (C₁-C₆)-alkyl, (C₁-C₆)-haloalkyl, (C₁-C₆)-haloalkoxy, (C₃-C₆)-cycloalkyl, (C₃-C₆)-halocycloalkyl, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, (C₁-C₄)-haloalkyl-S(O)ₙ, CHO, (C₁-C₄)-alkyloxycarbonyl and NH₂ (amino),

Z is the groups

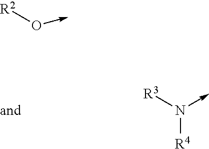

Y is halogen, cyano, isocyano, NO₂, NH₂ (amino), (C₁-C₆)-alkyl, (C₁-C₆)-haloalkyl, (C₁-C₆)-cyanoalkyl, (C₁-C₆)-hydroxyalkyl, (C₁-C₆)-alkoxy-(C₁-C₆)-alkyl, (C₃-C₆)-cycloalkyl, (C₁-C₄)-alkyloxycarbonyl, (C₁-C₆)-alkylcarbonyl, CHO, (C₃-C₆)-halocycloalkyl, (C₁-C₆)-alkoxy, (C₁-C₆)-haloalkoxy, (C₁-C₄)-alkyl-S(O)ₙ, (C₁-C₄)-haloalkyl-S(O)ₙ, (C₂-C₆)-alkynyl, (C₂-C₆)-haloalkynyl, (C₂-C₆)-alkenyl and (C₂-C₆)-haloalkenyl,

W is oxygen or sulfur,

R¹ is hydrogen, cyano, (Cₗ-C₆)-alkyl, (C₁-C₆)-haloalkyl, (C₁-C₆)-alkoxy, (C₁-C₆)-alkoxy-(C₁-C₆)-alkyl, (C₃-C₆)-cycloalkyl, (C₃-C₆)-halocycloalkyl, (C₂-C₆)-alkynyl, (C₂-C₆)-haloalkynyl, (C₂-C₆)-alkenyl and (C₂-C₆)-haloalkenyl,

R² is hydrogen, (C₁-C₁₀)-alkyl and (C₃-C₁₀)-cycloalkyl,

where the alkyl and cycloalkyl are unsubstituted or each independently substituted by m radicals selected from the group consisting of halogen, cyano, isocyano, nitro, NH₂ (amino), (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, CHO, (C₁-C₄)-alkyloxycarbonyl, heterocyclyl-(C₁-C₄)-alkyl, heteroaryl-(C₁-C₄)-alkyl and aryl-(C₁-C₄)-alkyl, where the aryl, heterocyclyl and heteroaryl are unsubstituted or each independently substituted by m radicals selected from the group consisting of halogen, (C₁-C₆)-alkyl and (C₁-C₆)-haloalkyl,

R³ is hydrogen and (C₁-C₁₂)-alkyl,

R⁴ is hydrogen, cyano, nitro, (C₁-C₁₂)-alkyl, (C₁-C₁₀)-haloalkyl, (C₂-C₁₀)-alkenyl, (C₃-C₁₀)-alkynyl, (C₁-C₁₀)-alkoxy-(C₁-C₁₀)-alkyl, (C₁-C₁₀)-alkoxy-(C₁-C₁₀)-alkyl, (C₁-C₄)-alkyl-S(O)ₙ—(C₁-C₄)-alkyl, (C₁-C₄)-haloalkyl-S(O)ₙ—(C₁-C₄)-alkyl, (C₃-C₁₀)-cycloalkyl, (C₃-C₁₀)-halocycloalkyl, hydroxy-(C₁-C₁₀)-alkylcarbonyl, amino-(C₁-C₁₀)-alkyl, (C₁-C₁₀)-alkoxycarbonyl-(C₁-C₁₀)-alkyl, (C₁-C₁₀)-cyanoalkyl, S(O)ₙR⁵, OR⁵, SO₂NR⁶R⁷, CO₂R⁸, COR⁸, NR⁶R⁸, NR⁶COR⁸, NR⁶CO₂R⁸, NR⁶SO₂R⁸, aryl, heteroaryl and heterocyclyl,

which are unsubstituted or each independently substituted by m radicals selected from the group consisting of hydrogen, halogen, cyano, nitro, OR⁵, S(O)ₙR⁵, SO₂NR⁶R⁷, CO₂R⁸, CONR⁶R⁸, COR⁶, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;

or

R³ and R⁴ together with the nitrogen atom to which they are bonded form a fully saturated or partly saturated 3- to 10-membered monocyclic or bicyclic ring optionally interrupted by heteroatoms and optionally having further substitution,

R⁵ is hydrogen, (C₁-C₈)-alkyl, (C₃-C₆)-cycloalkyl, (C₁-C₈)-haloalkyl and aryl,

R⁶ is hydrogen and R⁵,

R⁷ is hydrogen, (C₁-C₆)-alkyl, (C₃-C₆)-cycloalkyl, (C₃-C₄)-alkenyl, (C₃-C₄)-alkynyl and (C₁-C₁₀ alkylcarbonyl-(C₁-C₆)-alkyl,

R⁸ is hydrogen, (C₁-C₆)-alkyl, (C₃-C₆)-cycloalkyl, (C₃-C₄)-alkenyl, and (C₃-C₄)-alkynyl,
m is 0, 1 or 2, and n is 0, 1 or 2, excluding the compounds ethyl {[4-nitro-1-phenyl-5-(1H-tetrazol-5-yl)-1H-pyrazol-3-yl]sulfanyl}acetate CAS [1360702-86-8] and methyl {[5-(5-methyl-1,3,4-oxadiazol-2-yl)-4-nitro-1-phenyl-1H-pyrazol-3-yl]sulfanyl}acetate CAS [1360690-60-3].

The compounds of the general formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-toluenesulfonic acid, onto a basic group, for example amino, alkylamino, dialkylamino, piperidino, morpholino or pyridino. In such a case, these salts comprise the conjugate base of the acid as the anion. Suitable substituents in deprotonated form, for example sulfonic acids, particular sulfonamides or carboxylic acids, are capable of forming internal salts with groups, such as amino groups, which are themselves protonatable. Salts may also be formed by action of a base on compounds of the general formula (I). Suitable bases are, for example, organic amines such as trialkylamines, morpholine, piperidine and pyridine, and the hydroxides, carbonates and hydrogencarbonates of ammonium, alkali metals or alkaline earth metals, especially sodium hydroxide, potassium hydroxide, sodium carbonate, potassium carbonate, sodium hydrogencarbonate and potassium hydrogencarbonate. These salts are compounds in which the acidic hydrogen is replaced by an agriculturally suitable cation, for example metal salts, especially alkali metal salts or alkaline earth metal salts, in particular sodium and potassium salts, or else ammonium salts, salts with organic amines or quaternary ammonium salts, for example with cations of the formula [NR^{a}R^{b}R^{c}R^{d}]⁺in which R^{a} to R^{d} are each independently an organic radical, especially alkyl, aryl, arylalkyl or alkylaryl. Also suitable are alkylsulfonium and alkylsulfoxonium salts, such as (C₁-C₄)-trialkylsulfonium and (C₁-C₄)-trialkylsulfoxonium salts.

The inventive substituted [(1,4,5-trisubstituted 1H-pyrazol-3-yl)sulfanyl]acetic acid derivatives of the general formula (I) may, depending on external conditions such as pH, solvent and temperature, exist in various tautomeric structures, all of which are embraced by the general formula (I).

The compounds of the formula (I) used in accordance with the invention and salts thereof are referred to hereinafter as “compounds of the general formula (I)”.

The invention preferably provides compounds of the general formula (I) in which

Q¹ is the groups Q¹-1.1 to Q¹-6.5

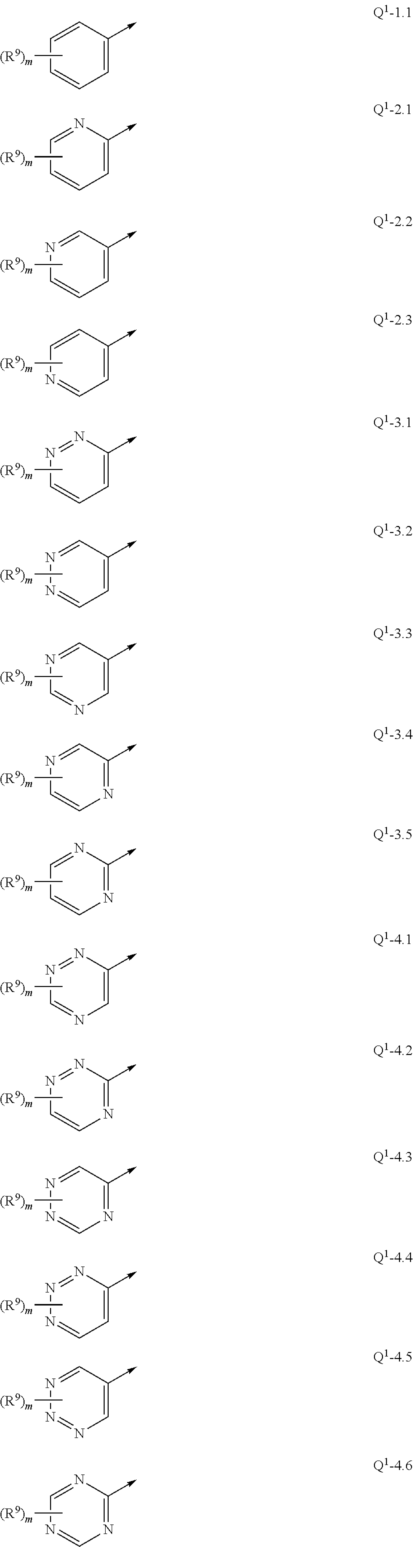

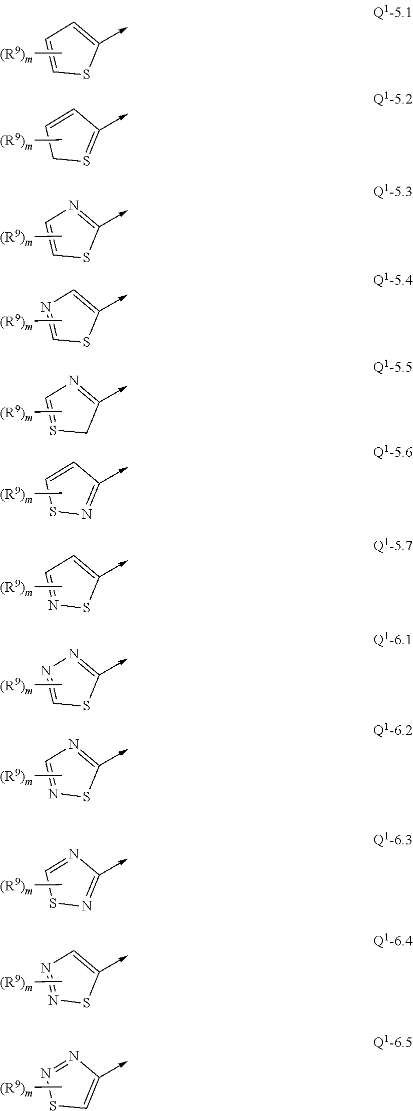

Q² is phenyl,

which is unsubstituted or in each case independently substituted by m radicals selected from the group consisting of hydrogen, halogen, cyano, isocyano, NO₂, (C₁-C₆)-alkyl, (C₁-C₆)-haloalkyl, (C₁-C₆)-haloalkoxy, (C₃-C₆)-cycloalkyl, (C₃-C₆)-halocycloalkyl, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, (C₁-C₄)-haloalkyl-S(O)ₙ, CHO, (C₁-C₄)-alkyloxycarbonyl and NH₂ (amino),

Z is the groups

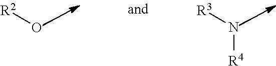

Y is halogen, cyano, isocyano, NO₂, NH₂ (amino), (C₁-C₆)-alkyl, (C₁-C₆)-haloalkyl, (C₁-C₆)-cyanoalkyl, (C₁-C₆)-hydroxyalkyl, (C₁-C₆)-alkoxy-(C₁-C₆)-alkyl, (C₃-C₆)-cycloalkyl, (C¹-C₄)-alkyloxycarbonyl, (C₁-C₆)-alkylcarbonyl, CHO, (C₃-C₆)-halocycloalkyl, (C₁-C₆)-alkoxy, (C₁-C₆)-haloalkoxy, (C₁-C₄)-alkyl-S(O)ₙ, (C₁-C₄)-haloalkyl-S(O)ₙ, (C₂-C₆)-alkynyl, (C₂-C₆)-haloalkynyl, (C₂-C₆)-alkenyl and (C₂-C₆)-haloalkenyl,

W is oxygen or sulfur,

R₁ is hydrogen, cyano, (C₁-C₆)-alkyl, (C₁-C₆)-haloalkyl, (C₁-C₆)-alkoxy, (C₁-C₆)-alkoxy-(C₁-C₆)-alkyl, (C₃-C₆)-cycloalkyl, (C₃-C₆)-halocycloalkyl, (C₂-C₆)-alkynyl, (C₂-C₆)-haloalkynyl, (C₂-C₆)-alkenyl and (C₂-C₆)-haloalkenyl,

R² is hydrogen, (C₁-C₁₀)-alkyl and (C₃-C₁₀)-cycloalkyl,

where the alkyl and cycloalkyl are unsubstituted or each independently substituted by m radicals selected from the group consisting of halogen, cyano, isocyano, nitro, NH₂ (amino), (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkenyl, (C₂-C₃)-haloalkynyl, (C₁-C₄)-alkyl-S(O)ₙ, CHO, (C₁-C₄)-alkyloxycarbonyl, heterocyclyl-(C₁-C₆)-alkyl, heteroaryl-(C₁-C₄)-alkyl and aryl-(C₁-C₄)-alkyl, where the aryl, heterocyclyl and heteroaryl are unsubstituted or each independently substituted by m radicals selected from the group consisting of halogen, (C₁-C₆)-alkyl and (C₁-C₆)-haloalkyl,

R³ is hydrogen and (C₁-C₁₀)-alkyl,

R⁴ is hydrogen, cyano, nitro, (C₁-C₁₀)-alkyl, (C₁-C₁₀)-haloalkyl, (C₂-C₁₀)-alkenyl, (C₃-C₁₀)-alkynyl, (C₁-C₁₀)-alkoxy-(C₁-C₁₀)-alkyl, (C₁-C₁₀ haloalkoxy-(C₁-C₁₀-alkyl, (C₁-C₄)-alkyl-S(O)ₙ—(C₁-C₄)-alkyl, (C₁-C₄)-haloalkyl-S(O)ₙ—(C₁-C₄)-alkyl, (C₃-C₁₀)-cycloalkyl, (C₃-C₁₀)-halocycloalkyl, hydroxy-(C₁-C₁₀)-alkylcarbonyl, amino-(C₁-C₁₀-alkyl, (C₁-C₁₀)-alkoxycarbonyl-(C₁-C₁₀-alkyl, (C₁-C₁₀)-cyanoalkyl, S(O)ₙR⁵, OR⁵, SO₂NR⁶R⁷, CO₂R⁸, COR⁶, NR⁶R⁸, NR⁶COR⁸, NR⁶CO₂R⁸, NR⁶SO₂R⁸; aryl, heteroaryl and heterocyclyl,

which are unsubstituted or each independently substituted by m radicals selected from the group consisting of hydrogen, halogen, cyano, nitro, OR⁵, S(O)ₙR⁵, SO₂NR⁶R⁷, CO₂R⁸, CONR⁶R⁸, COR⁶, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;
or R³ and R⁴ together with the nitrogen atom to which they are bonded form a fully saturated or partly saturated 3- to 10-membered monocyclic or bicyclic ring optionally interrupted by heteroatoms and optionally having further substitution,

R⁵ is hydrogen, (C₁-C₈)-alkyl, (C₃-C₆)-cycloalkyl, (C₁-C₈)-haloalkyl and aryl,

R⁶ is hydrogen and R⁵,

R⁷ is hydrogen, (C₁-C₆)-alkyl, (C₃-C₆)-cycloalkyl, (C₃-C₄)-alkenyl, (C₃-C₄)-alkynyl and (C₁-C₆)-alkylcarbonyl-(C₁-C₄)-alkyl,

R⁸ is hydrogen, (C₁-C₆)-alkyl, (C₃-C₆)-cycloalkyl, (C₃-C₄)-alkenyl and (C₃-C₄)-alkynyl,

R⁹ is hydrogen, halogen, cyano, isocyano, nitro, hydroxy, (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, CHO, (C₁-C₄)-alkyloxycarbonyl and NH₂ (amino),

m is 0, 1 and 2,

n is 0, 1 and 2.

The invention more preferably provides compounds of the general formula (I) in which

Q¹ is the groups Q¹-1.1 to Q¹-6.3,

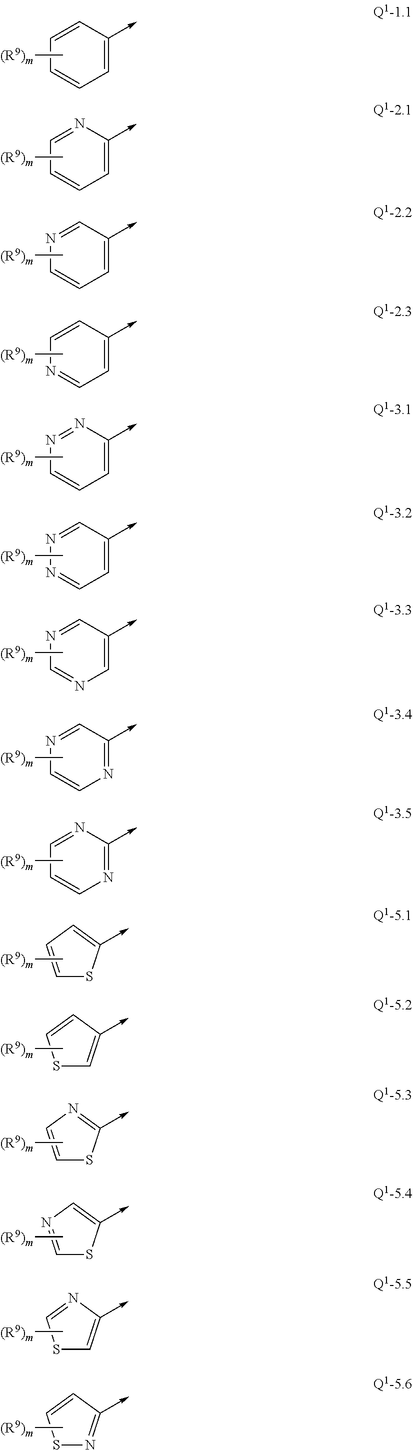

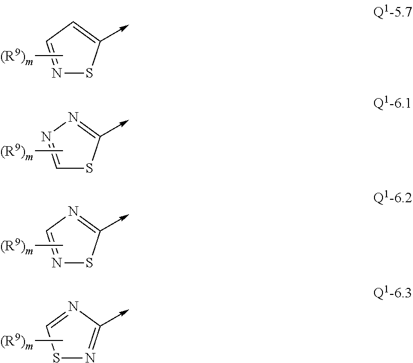

Q² is phenyl,

which is unsubstituted or in each case independently substituted by m radicals selected from the group consisting of hydrogen, halogen, cyano, (C₁-C₆)-alkyl, (C₁-C₆)-haloalkyl, (C₁-C₆)-haloalkoxy, (C₃-C₆)-cycloalkyl, (C₁-C₆)-alkoxy, (C₁-C₄)-alkyl-S(O)ₙ and (C₁-C₄)-haloalkyl-S(O)ₙ,

Z is the groups

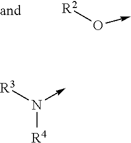

Y is halogen, cyano, isocyano, NO₂, (C₁-C₆)-alkyl, (C₁-C₆)-haloalkyl, (C₁-C₆)-alkoxy-(C₁-C₆)-alkyl, (C₁-C₄)-alkyloxycarbonyl, (C₁-C₆)-alkylcarbonyl, (C₁-C₆)-alkoxy, (C₁-C₆)-haloalkoxy, (Cₗ-C₄)-alkyl-S(O)ₙ, (C₁-C₄)-haloalkyl-S(O)ₙ, (C₂-C₆)-alkynyl, (C₂-C₆)-haloalkynyl, (C₂-C₆)-alkenyl and (C₂-C₆)-haloalkenyl,

W is oxygen,

R₁ is hydrogen, cyano, (Cₗ-C₆)-alkyl, (C₁-C₆)-haloalkyl, (C₁-C₆)-alkoxy, (C₁-C₄)-alkoxy-(C₁-C₄)-alkyl, (C₃-C₆)-cycloalkyl, (C₂-C₆)-alkynyl, (C₂-C₆)-haloalkynyl, (C₂-C₆)-alkenyl and (C₂-C₆)-haloalkenyl,

R² is hydrogen, (C₁-C₆)-alkyl and (C₃-C₆)-cycloalkyl, where the alkyl and cycloalkyl are unsubstituted or each independently substituted by m radicals selected from the group consisting of halogen, cyano, nitro, NH₂ (amino), (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (Cₗ-C₄)-alkyl-S(O)ₙ, CHO, (C₁-C₄)-alkyloxycarbonyl, heterocyclyl-(C₁-C₄)-alkyl, heteroaryl-(C₁-C₄)-alkyl and aryl-(C₁-C₄)-alkyl, where the aryl, heterocyclyl and heteroaryl are unsubstituted or each independently substituted by m radicals selected from the group consisting of halogen, (C₁-C₆)-alkyl and (C₁-C₆)-haloalkyl,

R³ is hydrogen and (C₁-C₆)-alkyl,

R⁴ is hydrogen, cyano, nitro, (C₁-C₆)-alkyl, (C₁-C₆)-haloalkyl, (C₂-C₆)-alkenyl, (C₃-C₆)-alkynyl, (C₁-C₆)-alkoxy-(C₁-C₆)-alkyl, (C₁-C₆)-haloalkoxy-(C₁-C₆)-alkyl, (C₁-C₄)-alkyl-S(O)ₙ—(C₁-C₄)-alkyl and (C₁-C₄)-haloalkyl-S(O)ₙ—(C₁-C₄)-alkyl, (C₃-C₆)-cycloalkyl, (C₃-C₆)-halocycloalkyl, hydroxy-(C₁-C₆)-alkylcarbonyl, amino-(C₁-C₆)-alkyl, (C₁-C₆)-alkoxycarbonyl-(C₁-C₆)-alkyl, S(O)ₙR⁵, OR⁵, SO₂NR⁶R⁷, CO₂R⁸, COR⁶, NR⁶R⁸, NR⁶COR⁸, NR⁶CO₂R⁸, NR⁶SO₂R⁸; aryl, heteroaryl and heterocyclyl, which are unsubstituted or each independently substituted by m radicals selected from the group consisting of hydrogen, halogen, cyano, nitro, OR⁵, S(O)ₙR⁵, SO₂NR⁶R⁷, CO₂R⁸, CONR⁶R⁸, COR⁶, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;

or R³ and R⁴ together with the nitrogen atom to which they are bonded form a fully saturated or partly saturated 3- to 10-membered monocyclic or bicyclic ring optionally interrupted by heteroatoms and optionally having further substitution,

R⁵ is hydrogen, (C₁-C₈)-alkyl, (C₃-C₆)-cycloalkyl, (C₁-C₈)-haloalkyl and aryl,

R⁶ is hydrogen and R⁵,

R⁷ is hydrogen, (C₁-C₆)-alkyl, (C₃-C₆)-cycloalkyl, (C₃-C₄)-alkenyl, and (C₃-C₄)-alkynyl,

R⁸ is hydrogen, (C₁-C₆)-alkyl, (C₃-C₆)-cycloalkyl, (C₃-C₄)-alkenyl, (C₃-C₄)-alkynyl and (C₁-C₁₀ alkylcarbonyl-(C₁-C₆)-alkyl,

R⁹ is hydrogen, halogen, cyano, isocyano, nitro, hydroxy, (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, CHO, (C₁-C₄)-alkyloxycarbonyl and NH₂ (amino),

m is 0, 1 and 2,

n is 0, 1 and 2.

The invention even more preferably provides compounds of the general formula (I) in which

Q¹ is the groups Q¹-1.1 to Q¹-6.3,

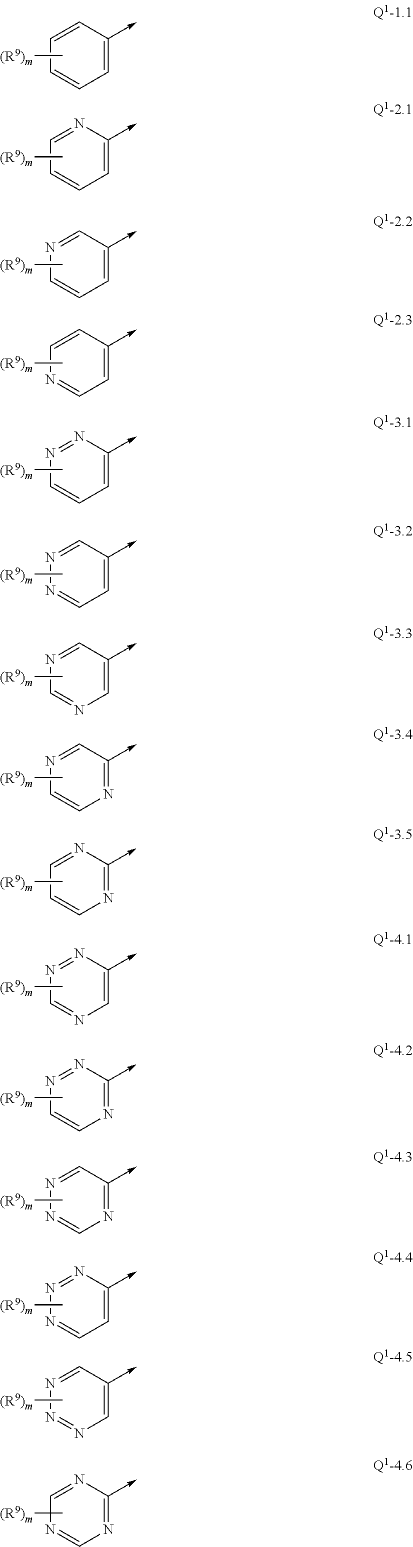

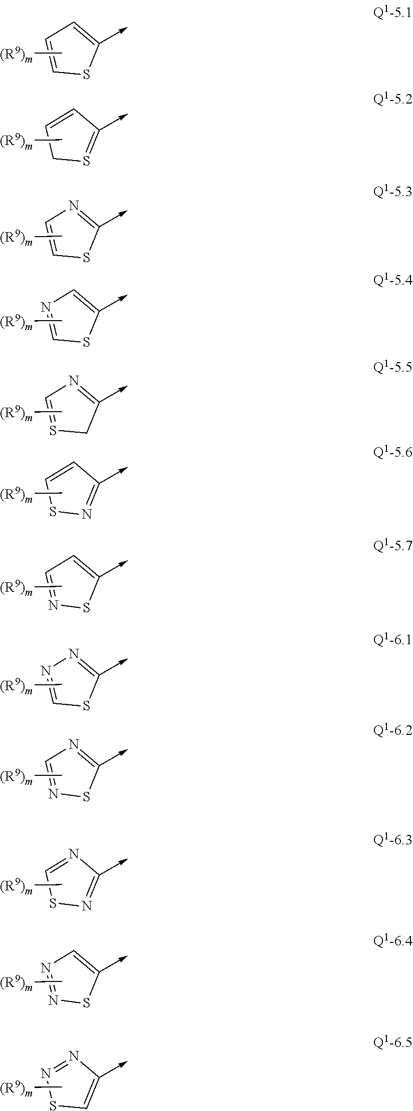

Q² is phenyl,

which is unsubstituted or in each case independently substituted by m radicals selected from the group consisting of hydrogen, fluorine, chlorine and bromine,

Z is the groups

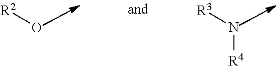

Y is fluorine, chlorine, bromine, cyano, NO₂, (C₁-C₂)-alkyl, (C₁-C₂)-haloalkyl, (C₁-C₂)-alkylcarbonyl, (C₁-C₂)-alkoxy, (C₁-C₂)-haloalkoxy, (C₁-C₂)-alkyl-S(O)ₙ, and (C₁-C₂)-haloalkyl-S(O)ₙ,

W is oxygen,

R¹ is hydrogen, (C₁-C₄)-alkyl, (C₁-C₄)-haloalkyl and (C₁-C₆)-alkoxy,

R² is hydrogen, (C₁-C₆)-alkyl and (C₃-C₆)-cycloalkyl,

where the alkyl and cycloalkyl are unsubstituted or each independently substituted by m radicals selected from the group consisting of halogen, cyano, nitro, NH₂ (amino), (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, CHO, (C₁-C₄)-alkyloxycarbonyl, heterocyclyl-(C₁-C₄)-alkyl, heteroaryl-(C₁-C₄)-alkyl and aryl-(C₁-C₄)-alkyl, where the aryl, heterocyclyl and heteroaryl are unsubstituted or each independently substituted by m radicals selected from the group consisting of halogen, (C₁-C₆)-alkyl and (C₁-C₆)-haloalkyl,

R³ is hydrogen and (C₁-C₆)-alkyl,

R⁴ is hydrogen, cyano, nitro, (C₁-C₆)-alkyl, (C₁-C₆)-haloalkyl, (C₂-C₆)-alkenyl, (C₃-C₆)-alkynyl, (C₁-C₆)-alkoxy-(C₁-C₆)-alkyl, (C₁-C₆)-haloalkoxy-(C₁-C₆)-alkyl, (C₁-C₄)-alkyl-S(O)ₙ—(C₁-C₄)-alkyl and (C₁-C₄)-haloalkyl-S(O)ₙ—(C₁-C₄)-alkyl, (C₃-C₆)-cycloalkyl, (C₃-C₆)-halocycloalkyl, hydroxy-(C₁-C₆)-alkylcarbonyl, amino-(C₁-C₆)-alkyl, (C₁-C₆)-alkoxycarbonyl-(Cₗ-C₆)-alkyl, S(O)ₙR⁵, OR⁵, SO₂NR⁶R⁷, CO₂R⁸, COR⁶, NR⁶R⁸, NR⁶COR⁸, NR⁶CO₂R⁸, NR⁶SO₂R⁸; aryl, heteroaryl and heterocyclyl,

which are unsubstituted or each independently substituted by m radicals selected from the group consisting of hydrogen, halogen, cyano, nitro, OR⁵, S(O)ₙR⁵, SO₂NR⁶R⁷, CO₂R⁸, CONR⁶R⁸, COR^{E}, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;

or R³ and R⁴ together with the nitrogen atom to which they are bonded form a fully saturated or partly saturated 3- to 10-membered monocyclic or bicyclic ring optionally interrupted by heteroatoms and optionally having further substitution,

R⁵ is hydrogen, (C₁-C₈)-alkyl, (C₃-C₆)-cycloalkyl, (C₁-C₈)-haloalkyl and aryl,

R⁶ is hydrogen and R⁵,

R⁷ is hydrogen, (C₁-C₆)-alkyl, (C₃-C₆)-cycloalkyl, (C₃-C₄)-alkenyl, (C₃-C₄)-alkynyl and (C₁-C₁₀ alkylcarbonyl-(C₁-C₆)-alkyl,

R⁸ is hydrogen, (C₁-C₆)-alkyl, (C₃-C₆)-cycloalkyl, (C₃-C₄)-alkenyl and (C₃-C₄)-alkynyl,

R⁹ is hydrogen, halogen, cyano, isocyano, nitro, hydroxy, (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, CHO, (C₁-C₄)-alkyloxycarbonyl and NH₂ (amino),

m is 0, 1 and 2,

n is 0, 1 and 2.

The invention especially preferably provides compounds of the general formula (I) in which Q¹ is the groups Q¹-1.1 to Q¹-5.7

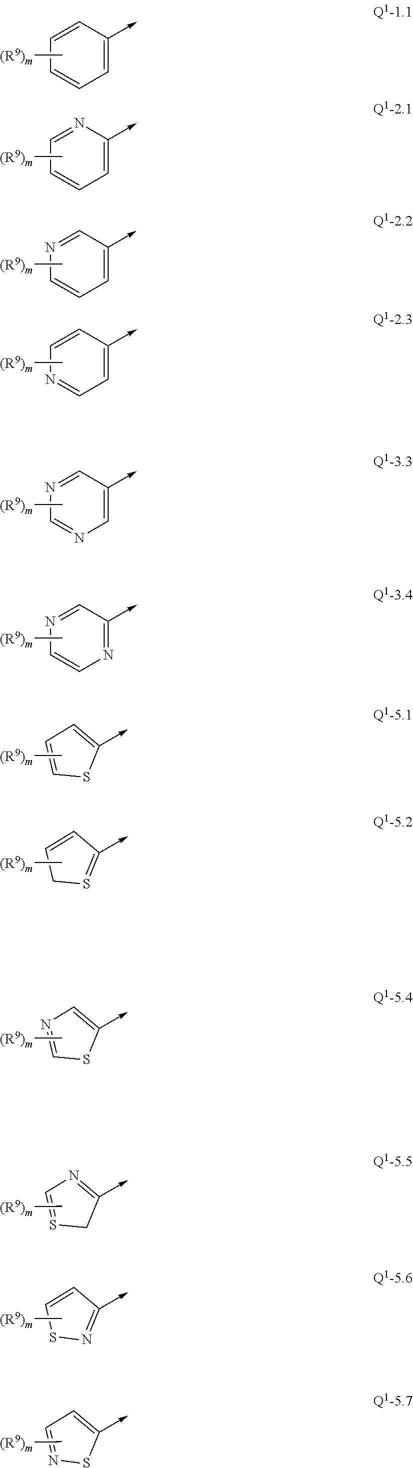

Q² is phenyl,

which is unsubstituted or in each case independently substituted by m radicals selected from the group consisting of hydrogen, fluorine, chlorine and bromine,

Z is the groups

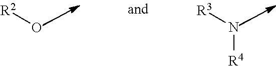

Y is fluorine, chlorine, bromine, cyano, NO₂, methyl, CF₃ and OCF₃,

W is oxygen,

R¹ is hydrogen, methyl and ethyl,

R² is hydrogen, methyl, ethyl, allyl, propargyl and PhCH₂,

R³ is hydrogen,

R⁴ is (C₁-C₆)-alkyl, S(O)ₙR⁵, OR⁵, SO₂NR⁶R⁷, CO₂R⁸, COR⁶,

which are unsubstituted or in each case independently of one another substituted by m radicals selected from the group consisting of hydrogen, OR⁵, S(O)ₙR⁵, SO₂NR⁶R⁷, CO₂R⁸, COR^{E}, NR⁶CO₂R⁸,

R⁵ is methyl, ethyl, CF₃, CH₂CF₃,

R⁶ is hydrogen and R⁵,

R⁷ is hydrogen, methyl, ethyl, and ethyl-2-ethanoyl,

R⁸ is methyl and ethyl,

R⁹ is hydrogen, fluorine, chlorine, bromine, cyano, hydroxy, methyl, ethyl, OCH₃, CF₃, and OCF₃,

m is 0, 1 and 2,

n is 0, 1 and 2.

The abovementioned general or preferred radical definitions apply both to the end products of the general formula (I) and, correspondingly, to the starting materials or the intermediates required in each case for the preparation. These radical definitions can be combined with one another as desired, i.e. including combinations between the given preferred ranges.

Of particular interest, primarily for reasons of higher herbicidal activity, better selectivity and/or better preparability, are inventive compounds of the general formula (I) given or salts thereof or the inventive use thereof in which individual radicals have one of the preferred meanings already specified or specified below, or in particular those in which one or more of the preferred meanings already specified or specified below occur in combination.

With regard to the compounds of the invention, the terms used above and further down will be elucidated. These are familiar to the person skilled in the art and especially have the definitions elucidated hereinafter:

Unless defined differently, names of chemical groups are generally to be understood such that attachment to the skeleton or the remainder of the molecule is via the structural element of the relevant chemical group mentioned last, i.e. for example in the case of (C₂-C₈)-alkenyloxy via the oxygen atom and in the case of heterocyclyl-(C₁-C₈)-alkyl or R¹²O(O)C—(C₁-C₈)-alkyl in each case via the carbon atom of the alkyl group.

According to the invention, “alkylsulfonyl”—alone or as part of a chemical group—refers to straight-chain or branched alkylsulfonyl, preferably having 1 to 8 or 1 to 6 carbon atoms, for example (but not limited to) (C₁-C₆)-alkylsulfonyl such as methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl.

According to the invention, “heteroarylsulfonyl” refers to optionally substituted pyridylsulfonyl, pyrimidinylsulfonyl, pyrazinylsulfonyl or optionally substituted polycyclic heteroarylsulfonyl, here in particular optionally substituted quinolinylsulfonyl, for example substituted by fluorine, chlorine, bromine, iodine, cyano, nitro, alkyl, haloalkyl, haloalkoxy, amino, alkylamino, alkylcarbonylamino, dialkylamino or alkoxy groups.

According to the invention, “alkylthio”—alone or as part of a chemical group—refers to straight-chain or branched S-alkyl, preferably having 1 to 8 or 1 to 6 carbon atoms, such as (C₁-C₁₀)—, (C₁-C₆)—or (C₁-C₄)-alkylthio, for example (but not limited to) (C₁-C₆)-alkylthio such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio.

According to the invention, “cycloalkylthio” denotes a cycloalkyl radical which is attached via a sulfur atom.

According to the invention, “alkylsulfinyl(alkyl-S(═O)—)”, unless defined differently elsewhere, denotes alkyl radicals which are bonded to the skeleton via—S(═O)—, such as (C₁-C₁₀)—, (C₁-C₆)—or (C₁-C₄)-alkylsulfinyl, for example (but not limited to) (C₁-C₆)-alkylsulfinyl such as methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl.

“Alkoxy” denotes an alkyl radical attached via an oxygen atom, for example (but not limited to) (C₁-C₆)-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. Alkenyloxy denotes an alkenyl radical attached via an oxygen atom, and alkynyloxy denotes an alkynyl radical attached via an oxygen atom, such as (C₂-C₁₀)—, (C₂-C₆)—or (C₂-C₄)-alkenoxy and (C₃-C₁₀)—, (C₃-C₆)—or (C₃-C₄)-alkynoxy.

“Cycloalkyloxy” denotes a cycloalkyl radical attached via an oxygen atom.

According to the invention, “alkylcarbonyl” (alkyl-C(═O)—), unless defined differently elsewhere, represents alkyl radicals bonded to the skeleton via —C(═O)—, such as (C₁-C₁₀)—, (C₁-C₆)—or (C₁-C₄)-alkylcarbonyl. The number of the carbon atoms here relates to the alkyl radical in the alkylcarbonyl group.

“Alkoxycarbonyl(alkyl-O—C(═O)—)”, unless defined differently elsewhere: alkyl radicals bonded to the skeleton via —O—C(═O)—, such as (C₁-C₁₀)—, (C₁-C₆)—or (C₁-C₄)-alkoxycarbonyl. The number of the carbon atoms here relates to the alkyl radical in the alkoxycarbonyl group. Analogously, “alkenyloxycarbonyl” and “alkynyloxycarbonyl”, unless defined differently elsewhere, in accordance with the invention, respectively represent alkenyl and alkynyl radicals bonded to the skeleton via —O—C(═O)—, such as (C₂-C₁₀)—, (C₂-C₆)—or (C₂-C₄)-alkenyloxycarbonyl and (C₃-C₁₀)—, (C₃-C₆)—or (C₃-C₄)-alkynyloxycarbonyl. The number of the carbon atoms here refers to the alkenyl or alkynyl radical in the alkenyloxycarbonyl or alkynyloxycarbonyl group.

According to the invention, the term “alkylcarbonyloxy” (alkyl-C(═O)—O—), unless defined differently elsewhere, represents alkyl radicals bonded to the skeleton via the oxygen of a carbonyloxy group (—C(═O)—O—), such as (C₁-C₁₀, (C₁-C₆)—or (C₁-C₄)-alkylcarbonyloxy. The number of the carbon atoms here relates to the alkyl radical in the alkylcarbonyloxy group.

The term “aryl” denotes an optionally substituted mono-, bi- or polycyclic aromatic system having preferably 6 to 14, especially 6 to 10, ring carbon atoms, for example phenyl, naphthyl, anthryl, phenanthrenyl and the like, preferably phenyl.

The term “optionally substituted aryl” also includes polycyclic systems, such as tetrahydronaphthyl, indenyl, indanyl, fluorenyl, biphenylyl, where the bonding site is on the aromatic system. In systematic terms, “aryl” is generally also encompassed by the term “optionally substituted phenyl”. Preferred aryl substituents here are, for example, hydrogen, halogen, alkyl, cycloalkyl, cycloalkylalkyl, cycloalkenyl, halocycloalkyl, alkenyl, alkynyl, aryl, arylalkyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, bisalkylaminoalkoxy, tris[alkyl]silyl, bis[alkyl]arylsilyl, bis[alkyl]alkylsilyl, tris[alkyl]silyl alkynyl, alkylalkynyl, cycloalkylalkynyl, haloalkylalkynyl, heterocyclyl-N-alkoxy, nitro, cyano, amino, alkylamino, bisalkylamino, alkylcarbonylamino, cycloalkylcarbonylamino, arylcarbonylamino, alkoxycarbonylamino, alkoxycarbonylalkylamino, arylalkoxycarbonylalkylamino, hydroxycarbonyl, alkoxycarbonyl, aminocarbonyl, alkylaminocarbonyl, cycloalkylaminocarbonyl, bisalkylaminocarbonyl, heteroarylalkoxy, arylalkoxy.

A heterocyclic radical (heterocyclyl) contains at least one heterocyclic ring (=carbocyclic ring in which at least one carbon atom has been replaced by a heteroatom, preferably by a heteroatom from the group of N, O, S, P) which is saturated, unsaturated, partly saturated or heteroaromatic and may be unsubstituted or substituted, in which case the bonding site is localized on a ring atom. If the heterocyclyl radical or the heterocyclic ring is optionally substituted, it may be fused to other carbocyclic or heterocyclic rings. In the case of optionally substituted heterocyclyl, polycyclic systems are also included, for example 8-azabicyclo[3.2.1]octanyl, 8-azabicyclo[2.2.2]octanyl or 1-azabicyclo[2.2.1]heptyl. Optionally substituted heterocyclyl also includes spirocyclic systems, for example 1-oxa-5-azaspiro[2.3]hexyl. Unless defined differently, the heterocyclic ring preferably contains 3 to 9 ring atoms, especially 3 to 6 ring atoms, and one or more, preferably 1 to 4, especially 1, 2 or 3, heteroatoms in the heterocyclic ring, preferably from the group of N, O and S, but no two oxygen atoms should be directly adjacent, for example with one heteroatom from the group of N, O and S: 1- or 2- or 3-pyrrolidinyl, 3,4-dihydro-2H-pyrrol-2- or -3-yl, 2,3-dihydro-1H-pyrrol-1- or -2- or -3- or -4- or -5-yl; 2,5-dihydro-1H-pyrrol-1- or -2- or -3-yl, 1- or 2- or 3- or 4-piperidinyl; 2,3,4,5-tetrahydropyridin-2- or -3- or -4- or -5-yl or -6-yl; 1,2,3,6-tetrahydropyridin-1- or -2- or -3- or -4- or -5- or -6-yl; 1,2,3,4-tetrahydropyridin-1- or -2- or -3- or -4- or -5- or -6-yl; 1,4-dihydropyridin-1- or -2- or -3- or -4-yl; 2,3-dihydropyridin-2- or -3- or -4- or -5- or -6-yl; 2,5-dihydropyridin-2- or -3- or -4- or -5- or -6-yl, 1- or 2- or 3- or 4-azepanyl; 2,3,4,5-tetrahydro-1H-azepin-1- or -2- or -3- or -4- or -5- or -6- or -7-yl; 2,3,4,7-tetrahydro-1H-azepin-1- or -2- or -3- or -4- or -5- or -6- or -7-yl; 2,3,6,7-tetrahydro-1H-azepin-1- or -2- or -3- or -4-yl; 3,4,5,6-tetrahydro-2H-azepin-2- or -3-or -4- or -5- or -6- or -7-yl; 4,5-dihydro-1H-azepin-1- or -2- or -3- or -4-yl; 2,5-dihydro-1H-azepin-1- or -2- or -3- or -4- or -5- or -6- or -7-yl; 2,7-dihydro-1H-azepin-1- or -2- or -3- or -4-yl; 2,3-dihydro-1H-azepin-1- or -2- or -3- or -4- or -5- or -6- or -7-yl; 3,4-dihydro-2H-azepin-2- or -3- or -4- or -5- or -6- or -7-yl; 3,6-dihydro-2H-azepin-2- or -3- or -4- or -5- or -6- or -7-yl; 5,6-dihydro-2H-azepin-2- or -3- or -4-or -5- or -6- or -7-yl; 4,5-dihydro-3H-azepin-2- or -3- or -4- or -5- or -6- or -7-yl; 1H-azepin-1- or -2- or -3- or -4- or -5- or -6- or -7-yl; 2H-azepin-2- or -3- or -4- or -5- or -6- or -7-yl; 3H-azepin-2- or -3- or or -5- or -6- or -7-yl; 4H-azepin-2- or -3- or -4- or -5- or -6- or -7-yl, 2- or 3-oxolanyl(=2- or 3-tetrahydrofuranyl); 2,3-dihydrofuran-2- or -3- or -4- or -5-yl; 2,5-dihydrofuran-2- or -3-yl, 2- or 3- or 4-oxanyl(=2- or 3- or 4-tetrahydropyranyl); 3,4-dihydro-2H-pyran-2- or -3- or -4- or -5- or -6-yl; 3,6-dihydro-2H-pyran-2- or -3- or -4- or -5- or -6-yl; 2H-pyran-2- or -3- or -4- or -5- or -6-yl; 4H-pyran-2- or -3- or -4-yl, 2- or 3- or 4-oxepanyl; 2,3,4,5-tetrahydrooxepin-2- or -3- or -4- or -5- or -6- or -7-yl; 2,3,4,7-tetrahydrooxepin-2- or -3- or -4- or -5- or -6- or -7-yl; 2,3,6,7-tetrahydrooxepin-2- or -3- or -4-yl; 2,3-dihydrooxepin-2- or -3- or -4- or -5- or -6- or -7-yl; 4,5-dihydrooxepin-2- or -3- or -4-yl; 2,5-dihydrooxepin-2- or -3- or -4- or -5- or -6- or -7-yl; oxepin-2- or -3- or -4- or -5- or -6- or -7-yl; 2- or 3-tetrahydrothiophenyl; 2,3-dihydrothiophen-2- or -3- or -4- or -5-yl; 2,5-dihydrothiophen-2- or -3-yl; tetrahydro-2H-thiopyran-2- or -3- or -4-yl; 3,4-dihydro-2H-thiopyran-2- or -3- or -4- or -5- or -6-yl; 3,6-dihydro-2H-thiopyran-2- or -3- or -4- or -5- or -6-yl; 2H-thiopyran-2- or -3- or -4- or -5- or -6-yl; 4H-thiopyran-2- or -3- or -4-yl. Preferred 3-membered and 4-membered heterocycles are, for example, 1- or 2-aziridinyl, oxiranyl, thiiranyl, 1- or 2- or 3-azetidinyl, 2- or 3-oxetanyl, 2- or 3-thietanyl, 1,3-dioxetan-2-yl. Further examples of “heterocyclyl” are a partly or fully hydrogenated heterocyclic radical having two heteroatoms from the group of N, O and S, for example 1- or 2- or 3- or 4-pyrazolidinyl; 4,5-dihydro-3H-pyrazol-3- or 4- or 5-yl; 4,5-dihydro-1H-pyrazol-1- or 3- or 4- or 5-yl; 2,3-dihydro-1H-pyrazol-1- or 2- or 3- or 4- or 5-yl; 1- or 2- or 3- or 4- imidazolidinyl; 2,3-dihydro-1H-imidazol-1- or 2- or 3- or 4-yl; 2,5-dihydro-1H-imidazol-1- or 2- or 4- or 5-yl; 4,5-dihydro-1H-imidazol-1- or 2- or 4- or 5-yl; hexahydropyridazin-1- or 2- or 3- or 4-yl; 1,2,3,4-tetrahydropyridazin-1- or 2- or 3- or 4- or 5- or 6-yl; 1,2,3,6-tetrahydropyridazin-1- or 2- or 3- or 4- or 5- or 6-yl; 1,4,5,6-tetrahydropyridazin-1- or 3- or 4- or 5- or 6-yl; 3,4,5,6-tetrahydropyridazin-3- or 4- or 5-yl; 4,5-dihydropyridazin-3- or 4-yl; 3,4-dihydropyridazin-3- or 4- or 5- or 6-yl; 3,6-dihydropyridazin-3- or 4-yl; 1,6-dihydropyriazin-1- or 3- or 4- or 5- or 6-yl; hexahydropyrimidin-1- or 2- or 3- or 4-yl; 1,4,5,6-tetrahydropyrimidin-1- or 2- or 4- or 5- or 6-yl; 1,2,5,6-tetrahydropyrimidin-1- or 2- or 4- or 5- or 6-yl; 1,2,3,4-tetrahydropyrimidin-1- or 2- or 3- or 4- or 5- or 6-yl; 1,6-dihydropyrimidin-1- or 2- or 4- or 5- or 6-yl; 1,2-dihydropyrimidin-1- or 2- or 4- or 5- or 6-yl; 2,5-dihydropyrimidin-2- or 4- or 5-yl; 4,5-dihydropyrimidin-4- or 5- or 6-yl; 1,4-dihydropyrimidin-1- or 2- or 4- or 5- or 6-yl; 1- or 2- or 3-piperazinyl; 1,2,3,6-tetrahydropyrazin-1- or 2-or 3- or 5- or 6-yl; 1,2,3,4-tetrahydropyrazin-1- or 2- or 3- or 4- or 5- or 6-yl; 1,2-dihydropyrazin-1- or 2-or 3- or 5- or 6-yl; 1,4-dihydropyrazin-1- or 2- or 3-yl; 2,3-dihydropyrazin-2- or 3- or 5- or 6-yl; 2,5-dihydropyrazin-2- or 3-yl; 1,3-dioxolan-2- or 4- or 5-yl; 1,3-dioxol-2- or 4-yl; 1,3-dioxan-2- or 4- or 5-yl; 4H-1,3-dioxin-2- or 4- or 5- or 6-yl; 1,4-dioxan-2- or 3- or 5- or 6-yl; 2,3-dihydro-1,4-dioxin-2- or 3- or 5- or 6-yl; 1,4-dioxin-2- or 3-yl; 1,2-dithiolan-3- or 4-yl; 3H-1,2-dithiol-3- or 4- or 5-yl; 1,3-dithiolan-2-or 4-yl; 1,3-dithiol-2- or 4-yl; 1,2-dithian-3- or 4-yl; 3,4-dihydro-1,2-dithiin-3- or 4- or 5- or 6-yl; 3,6-dihydro-1,2-dithiin-3- or 4-yl; 1,2-dithiin-3- or 4-yl; 1,3-dithian-2- or 4- or 5-yl; 4H-1,3-dithiin-2- or 4-or 5- or 6-yl; isoxazolidin-2- or 3- or 4- or 5-yl; 2,3-dihydroisoxazol-2- or 3- or 4- or 5-yl; 2,5-dihydroisoxazol-2- or 3- or 4- or 5-yl; 4,5-dihydroisoxazol-3- or 4- or 5-yl; 1,3-oxazolidin-2- or 3- or 4-or 5-yl; 2,3-dihydro-1,3-oxazol-2- or 3- or 4- or 5-yl; 2,5-dihydro-1,3-oxazol-2- or 4- or 5-yl; 4,5-dihydro-1,3-oxazol-2- or 4- or 5-yl; 1,2-oxazinan-2- or 3- or 4- or 5- or 6-yl; 3,4-dihydro-2H-1,2-oxazin-2- or 3-or 4- or 5- or 6-yl; 3,6-dihydro-2H-1,2-oxazin-2- or 3- or 4- or 5- or 6-yl; 5,6-dihydro-2H-1,2-oxazin or 3- or 4- or 5- or 6-yl; 5,6-dihydro-4H-1,2-oxazin-3- or 4- or 5- or 6-yl; 2H-1,2-oxazin-2- or 3- or 4- or 5- or 6-yl; 6H-1,2-oxazin-3- or 4- or 5- or 6-yl; 4H-1,2-oxazin-3- or 4- or 5- or 6-yl; 1,3-oxazinan-2- or 3-or 4- or 5- or 6-yl; 3,4-dihydro-2H-1,3-oxazin-2- or 3- or 4- or 5- or 6-yl; 3,6-dihydro-2H-1,3-oxazin or 3- or 4- or 5- or 6-yl; 5,6-dihydro-2H-1,3-oxazin-2- or 4- or 5- or 6-yl; 5,6-dihydro-4H-1,3-oxazin or 4- or 5- or 6-yl; 2H-1,3-oxazin-2- or 4- or 5- or 6-yl; 6H-1,3-oxazin-2- or 4- or 5- or 6-yl; 4H-1,3-oxazin-2- or 4- or 5- or 6-yl; morpholin-2- or 3- or 4-yl; 3,4-dihydro-2H-1,4-oxazin-2- or 3- or 4- or 5- or 6-yl; 3,6-dihydro-2H-1,4-oxazin-2- or 3- or 5- or 6-yl; 2H-1,4-oxazin-2- or 3- or 5- or 6-yl; 4H-1,4-oxazin-2- or 3-yl; 1,2-oxazepan-2- or 3- or 4- or 5- or 6- or 7-yl; 2,3,4,5-tetrahydro-1,2-oxazepin-2- or 3- or 4- or 5- or 6- or 7-yl; 2,3,4,7-tetrahydro-1,2-oxazepin-2- or 3- or 4- or 5- or 6- or 7-yl; 2,3,6,7-tetrahydro-1,2-oxazepin-2- or 3- or 4- or 5- or 6- or 7-yl; 2,5,6,7-tetrahydro-1,2-oxazepin-2- or 3- or 4- or 5- or 6- or 7-yl; 4,5,6,7-tetrahydro-1,2-oxazepin-3- or 4- or 5- or 6- or 7-yl; 2,3-dihydro-1,2-oxazepin-2- or 3- or 4- or 5- or 6- or 7-yl; 2,5-dihydro-1,2-oxazepin-2- or 3- or 4- or 5- or 6- or 7-yl; 2,7-dihydro-1,2-oxazepin-2-or 3- or 4- or 5- or 6- or 7-yl; 4,5-dihydro-1,2-oxazepin-3- or 4- or 5- or 6- or 7-yl; 4,7-dihydro-1,2-oxazepin-3- or 4- or 5- or 6- or 7-yl; 6,7-dihydro-1,2-oxazepin-3- or 4- or 5- or 6- or 7-yl; 1,2-oxazepin-3- or 4- or 5- or 6- or 7-yl; 1,3-oxazepan-2- or 3- or 4- or 5- or 6- or 7-yl; 2,3,4,5-tetrahydro-1,3-oxazepin-2- or 3- or 4- or 5- or 6- or 7-yl; 2,3,4,7-tetrahydro-1,3-oxazepin-2- or 3- or 4- or 5- or 6- or 7-yl; 2,3,6,7-tetrahydro-1,3-oxazepin-2- or 3- or 4- or 5- or 6- or 7-yl; 2,5,6,7-tetrahydro-1,3-oxazepin-2- or 4- or 5- or 6- or 7-yl; 4,5,6,7-tetrahydro-1,3-oxazepin-2- or 4- or 5- or 6- or 7-yl; 2,3-dihydro-1,3-oxazepin-2- or 3-or 4- or 5- or 6- or 7-yl; 2,5-dihydro-1,3-oxazepin-2- or 4- or 5- or 6- or 7-yl; 2,7-dihydro-1,3-oxazepin-2- or 4- or 5- or 6- or 7-yl; 4,5-dihydro-1,3-oxazepin-2- or 4- or 5- or 6- or 7-yl; 4,7-dihydro-1,3-oxazepin-2- or 4- or 5- or 6- or 7-yl; 6,7-dihydro-1,3-oxazepin-2- or 4- or 5- or 6- or 7-yl; 1,3-oxazepin-2- or 4- or 5- or 6- or 7-yl; 1,4-oxazepan-2- or 3- or 5- or 6- or 7-yl; 2,3,4,5-tetrahydro-1,4-oxazepin-2- or 3- or 4- or 5- or 6- or 7-yl; 2,3,4,7-tetrahydro-1,4-oxazepin-2- or 3- or 4- or 5- or 6- or 7-yl; 2,3,6,7-tetrahydro-1,4-oxazepin-2- or 3- or 5- or 6- or 7-yl; 2,5,6,7-tetrahydro-1,4-oxazepin-2- or 3- or 5- or 6- or 7-yl; 4,5,6,7-tetrahydro-1,4-oxazepin-2- or 3- or 4- or 5- or 6- or 7-yl; 2,3-dihydro-1,4-oxazepin-2- or 3- or 5- or 6- or 7-yl; 2,5-dihydro-1,4-oxazepin-2- or 3- or 5- or 6- or 7-yl; 2,7-dihydro-1,4-oxazepin-2- or 3- or 5- or 6-or 7-yl; 4,5-dihydro-1,4-oxazepin-2- or 3- or 4- or 5- or 6- or 7-yl; 4,7-dihydro-1,4-oxazepin-2- or 3- or 4- or 5- or 6- or 7-yl; 6,7-dihydro-1,4-oxazepin-2- or 3- or 5- or 6- or 7-yl; 1,4-oxazepin-2- or 3- or 5- or 6- or 7-yl; isothiazolidin-2- or 3- or 4- or 5-yl; 2,3-dihydroisothiazol-2- or 3- or 4- or 5-yl; 2,5-dihydroisothiazol-2- or 3- or 4- or 5-yl; 4,5-dihydroisothiazol-3- or 4- or 5-yl; 1,3-thiazolidin-2- or 3- or 4- or 5-yl; 2,3-dihydro-1,3-thiazol-2- or 3- or 4- or 5-yl; 2,5-dihydro-1,3-thiazol-2- or 4- or 5-yl; 4,5-dihydro-1,3-thiazol-2- or 4- or 5-yl; 1,3-thiazinan-2- or 3- or 4- or 5- or 6-yl; 3,4-dihydro-2H-1,3-thiazin-2- or 3- or 4- or 5- or 6-yl; 3,6-dihydro-2H-1,3-thiazin-2- or 3- or 4- or 5- or 6-yl; 5,6-dihydro-2H-1,3-thiazin-2- or 4- or 5- or 6-yl; 5,6-dihydro-4H-1,3-thiazin-2- or 4- or 5- or 6-yl; 2H-1,3-thiazin-2- or 4- or 5- or 6-yl; 6H-1,3-thiazin-2- or 4- or 5- or 6-yl; 4H-1,3-thiazin-2- or 4- or 5- or 6-yl. Further examples of “heterocyclyl” are a partly or fully hydrogenated heterocyclic radical having 3 heteroatoms from the group of N, O and S, for example 1,4,2-dioxazolidin-2- or 3- or 5-yl; 1,4,2-dioxazol-3- or 5-yl; 1,4,2-dioxazinan-2- or -3- or 5- or 6-yl; 5,6-dihydro-1,4,2-dioxazin-3- or 5- or 6-yl; 1,4,2-dioxazin-3- or 5- or 6-yl; 1,4,2-dioxazepan-2- or 3- or 5- or 6- or 7-yl; 6,7-dihydro-5H-1,4,2-dioxazepin-3- or 5- or 6- or 7-yl; 2,3-dihydro-7H-1,4,2-dioxazepin-2- or 3- or 5- or 6- or 7-yl; 2,3-dihydro-5H-1,4,2-dioxazepin-2- or 3- or 5- or 6- or 7-yl; 5H-1,4,2-dioxazepin-3- or 5- or 6- or 7-yl; 7H-1,4,2-dioxazepin-3- or 5- or 6- or 7-yl. Structural examples of heterocycles which are optionally substituted further are also listed below:

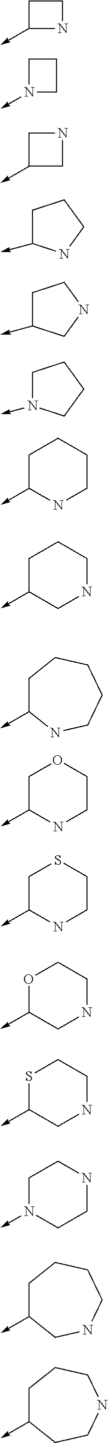

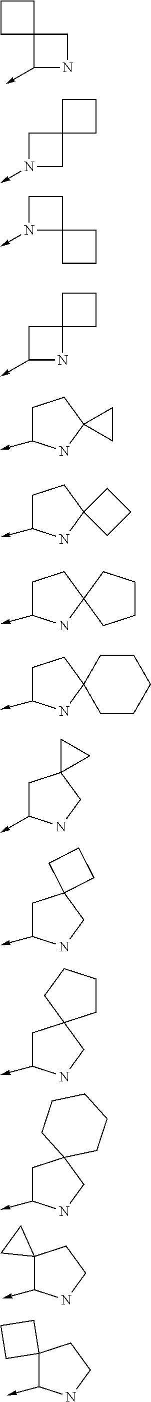

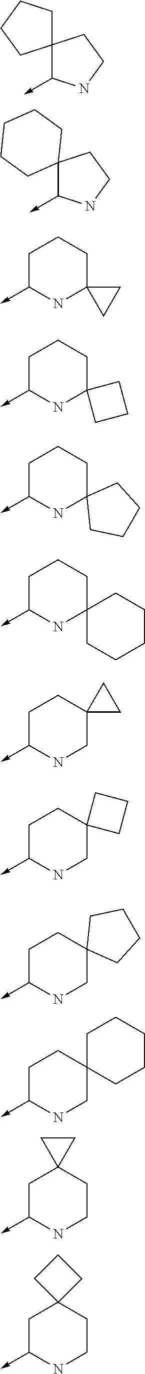

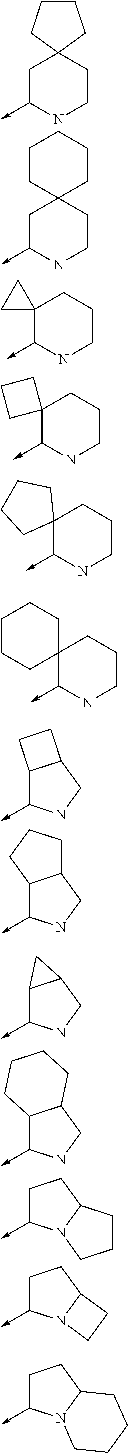

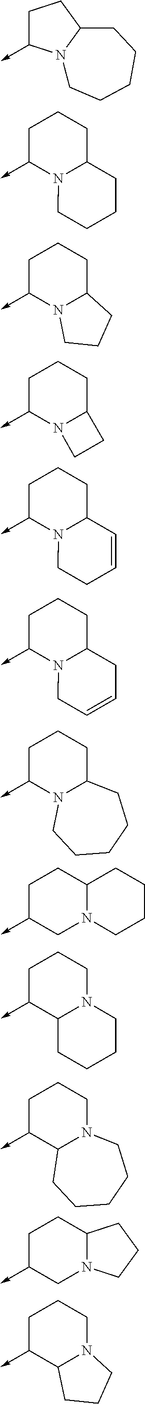

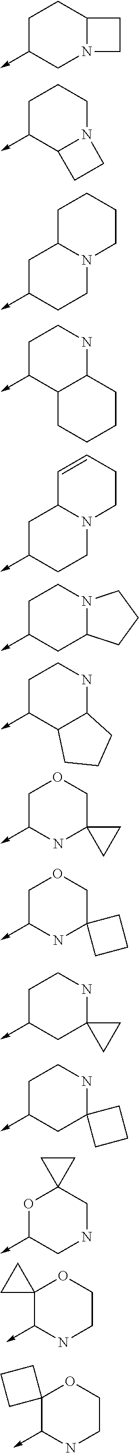

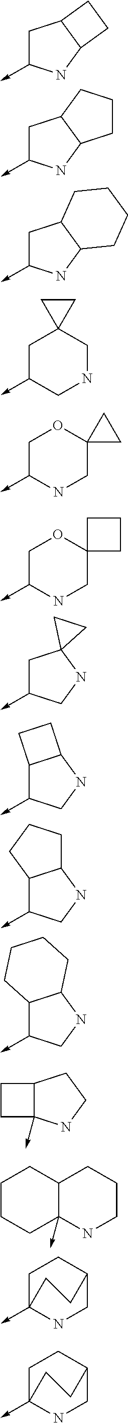

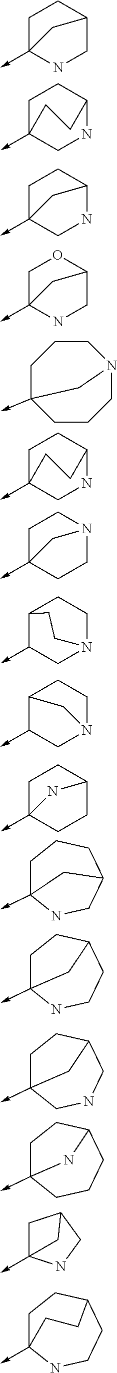

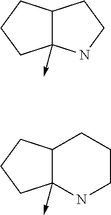

The heterocycles listed above are preferably substituted, for example, by hydrogen, halogen, alkyl, haloalkyl, hydroxyl, alkoxy, cycloalkoxy, aryloxy, alkoxyalkyl, alkoxyalkoxy, cycloalkyl, halocycloalkyl, aryl, arylalkyl, heteroaryl, heterocyclyl, alkenyl, alkylcarbonyl, cycloalkylcarbonyl, arylcarbonyl, heteroarylcarbonyl, alkoxycarbonyl, hydroxycarbonyl, cycloalkoxycarbonyl, cycloalkylalkoxycarbonyl, alkoxycarbonylalkyl, arylalkoxycarbonyl, arylalkoxycarbonylalkyl, alkynyl, alkynylalkyl, alkylalkynyl, trisalkylsilylalkynyl, nitro, amino, cyano, haloalkoxy, haloalkylthio, alkylthio, hydrothio, hydroxyalkyl, oxo, heteroarylalkoxy, arylalkoxy, heterocyclylalkoxy, heterocyclylalkylthio, heterocyclyloxy, heterocyclylthio, heteroaryloxy, bisalkylamino, alkylamino, cycloalkylamino, hydroxycarbonylalkylamino, alkoxycarbonylalkylamino, arylalkoxycarbonylalkylamino, alkoxycarbonylalkyl(alkyl)amino, aminocarbonyl, alkylaminocarbonyl, bisalkylaminocarbonyl, cycloalkylaminocarbonyl, hydroxycarbonylalkylaminocarbonyl, alkoxycarbonylalkylaminocarbonyl, arylalkoxycarbonylalkylaminocarbonyl.

When a base structure is substituted “by one or more radicals” from a list of radicals (=group) or a generically defined group of radicals, this in each case includes simultaneous substitution by a plurality of identical and/or structurally different radicals.

In the case of a partly or fully saturated nitrogen heterocycle, this may be joined to the remainder of the molecule either via carbon or via the nitrogen.

Suitable substituents for a substituted heterocyclic radical are the substituents specified further down, and additionally also oxo and thioxo. The oxo group as a substituent on a ring carbon atom is then, for example, a carbonyl group in the heterocyclic ring. As a result, lactones and lactams are preferably also included. The oxo group may also occur on the ring heteroatoms, which may exist in different oxidation states, for example in the case of N and S, and in that case form, for example, the divalent —N(O)—, —S(O)— (also SO for short) and —S(O)₂— (also SO₂ for short) groups in the heterocyclic ring. In the case of —N(O)—and —S(O)— groups, both enantiomers in each case are included.

According to the invention, the expression “heteroaryl” represents heteroaromatic compounds, i.e. fully unsaturated aromatic heterocyclic compounds, preferably 5- to 7-membered rings having 1 to 4, preferably 1 or 2, identical or different heteroatoms, preferably 0, S or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepinyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazinyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepinyl, thiepinyl, 1,2,4-triazolonyl and 1,2,4-diazepinyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The heteroaryl groups of the invention may also be substituted by one or more identical or different radicals. If two adjacent carbon atoms are part of a further aromatic ring, the systems are fused heteroaromatic systems, such as benzofused or polyannelated heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, 2H-indazol-7-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

The term “halogen” denotes, for example, fluorine, chlorine, bromine or iodine. If the term is used for a radical, “halogen” denotes, for example, a fluorine, chlorine, bromine or iodine atom.

According to the invention, “alkyl” means a straight-chain or branched open-chain, saturated hydrocarbon radical which is optionally mono- or polysubstituted, and in the latter case is referred to as “substituted alkyl”. Preferred substituents are halogen atoms, alkoxy, haloalkoxy, cyano, alkylthio, haloalkylthio, amino or nitro groups, particular preference being given to methoxy, methyl, fluoroalkyl, cyano, nitro, fluorine, chlorine, bromine or iodine. The prefix “bis” also includes the combination of different alkyl radicals, e.g. methyl(ethyl) or ethyl(methyl).

“Haloalkyl”, “-alkenyl” and “-alkynyl” respectively denote alkyl, alkenyl and alkynyl partly or fully substituted by identical or different halogen atoms, for example monohaloalkyl such as CH₂CH₂Cl, CH₂CH₂Br, CHClCH₃, CH₂Cl, CH₂F; perhaloalkyl such as CCl₃, CClF₂, CFC12, CF₂CClF₂, CF₂CClFCF₃; polyhaloalkyl such as CH₂CHFC1, CF₂CClFH, CF₂CBrFH, CH₂CF₃; the term perhaloalkyl also encompasses the term perfluoroalkyl.

“Partly fluorinated alkyl” denotes a straight-chain or branched, saturated hydrocarbon which is mono- or polysubstituted by fluorine, where the fluorine atoms in question may be present as substituents on one or more different carbon atoms of the straight-chain or branched hydrocarbon chain, for example CHFCH₃, CH₂CH₂F, CH₂CH₂CF₃, CHF2, CH₂F, CHFCF₂CF₃.

“Partly fluorinated haloalkyl” denotes a straight-chain or branched, saturated hydrocarbon which is substituted by different halogen atoms with at least one fluorine atom, where any other halogen atoms optionally present are selected from the group consisting of fluorine, chlorine or bromine, iodine. The corresponding halogen atoms may be present as substituents on one or more different carbon atoms of the straight-chain or branched hydrocarbon chain. Partly fluorinated haloalkyl also includes full substitution of the straight or branched chain by halogen including at least one fluorine atom.

“Haloalkoxy” is, for example, OCF₃, OCHF₂, OCH₂F, OCF₂CF₃, OCH₂CF₃ and OCH₂CH₂C₁; this applies correspondingly to haloalkenyl and other halogen-substituted radicals.

The expression “(C₁-C₄)-alkyl” mentioned here by way of example is a brief notation for straight-chain or branched alkyl having one to 4 carbon atoms according to the range stated for carbon atoms, i.e. encompasses the methyl, ethyl, 1-propyl, 2-propyl, 1-butyl, 2-butyl, 2-methylpropyl or tert-butyl radicals. General alkyl radicals with a larger specified range of carbon atoms, e.g. “(C₁-C₆)-alkyl”, correspondingly also encompass straight-chain or branched alkyl radicals with a greater number of carbon atoms, i.e. according to the example also the alkyl radicals having 5 and 6 carbon atoms.

Unless stated specifically, preference is given to the lower carbon skeletons, for example having from 1 to 6 carbon atoms, or having from 2 to 6 carbon atoms in the case of unsaturated groups, in the case of the hydrocarbon radicals such as alkyl, alkenyl and alkynyl radicals, including in composite radicals. Alkyl radicals, including in composite radicals such as alkoxy, haloalkyl, etc., are, for example, methyl, ethyl, n-propyl or i-propyl, n-, i-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

The term “alkenyl” also includes, in particular, straight-chain or branched open-chain hydrocarbon radicals having more than one double bond, such as 1,3-butadienyl and 1,4-pentadienyl, but also allenyl or cumulenyl radicals having one or more cumulated double bonds, for example allenyl(1,2-propadienyl), 1,2-butadienyl and 1,2,3-pentatrienyl. Alkenyl denotes, for example, vinyl which may optionally be substituted by further alkyl radicals, for example (but not limited thereto) (C₂-C₆)-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “alkynyl” also includes, in particular, straight-chain or branched open-chain hydrocarbon radicals having more than one triple bond, or else having one or more triple bonds and one or more double bonds, for example 1,3-butatrienyl or 3-penten-1-yn-1-yl. (C₂-C₆)-Alkynyl denotes, for example, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl pentynyl, 1,1-dimethyl-2-butynyl, 1,1-dimethyl-3-butynyl, 1,2-dimethyl-3-butynyl, 2,2-dimethyl butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

The term “cycloalkyl” refers to a carbocyclic saturated ring system having preferably 3-8 ring carbon atoms, for example cyclopropyl, cyclobutyl, cyclopentyl or cyclohexyl, which optionally has further substitution, preferably by hydrogen, alkyl, alkoxy, cyano, nitro, alkylthio, haloalkylthio, halogen, alkenyl, alkynyl, haloalkyl, amino, alkylamino, bisalkylamino, alkoxycarbonyl, hydroxycarbonyl, arylalkoxycarbonyl, aminocarbonyl, alkylaminocarbonyl, cycloalkylaminocarbonyl. In the case of optionally substituted cycloalkyl, cyclic systems with substituents are included, also including substituents with a double bond on the cycloalkyl radical, for example an alkylidene group such as methylidene. In the case of optionally substituted cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[1.1.1]pentan-1- yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl and adamantan-2-yl, but also systems such as 1,1′-bi(cyclopropyl)-1-yl, 1,1′-bi(cyclopropyl)-2-yl, for example. The term “(C₃-C₇)-cycloalkyl” is a brief notation for cycloalkyl having three to 7 carbon atoms, corresponding to the range specified for carbon atoms.

In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl.

“Cycloalkenyl” denotes a carbocyclic, nonaromatic, partly unsaturated ring system having preferably 4-8 carbon atoms, e.g. 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

The term “alkylidene”, also, for example, in the form (C₁-C₁₀)-alkylidene, denotes the radical of a straight-chain or branched open-chain hydrocarbon radical which is attached via a double bond. Possible bonding sites for alkylidene are naturally only positions on the base structure where two hydrogen atoms can be replaced by the double bond; radicals are, for example, ═CH₂, ═CH—CHs, ═C(CH₃)—CH₃, ═C(CH₃)—C₂H₅ or ═C(C₂H₅)—C₂H₅Cycloalkylidene denotes a carbocyclic radical bonded via a double bond.

“Alkoxyalkyl” represents an alkoxy radical bonded via an alkyl group and “alkoxyalkoxy” denotes an alkoxyalkyl radical bonded via an oxygen atom, for example (but not limited to) methoxymethoxy, methoxyethoxy, ethoxyethoxy, methoxy-n-propyloxy.

“Alkylthioalkyl” represents an alkylthio radical bonded via an alkyl group and “alkylthioalkylthio” denotes an alkylthioalkyl radical bonded via an oxygen atom.

“Arylalkoxyalkyl” represents an aryloxy radical bonded via an alkyl group and “heteroaryloxyalkyl” denotes a heteroaryloxy radical bonded via an alkyl group.

“Haloalkoxyalkyl” represents a bonded haloalkoxy radical and “haloalkylthioalkyl” denotes a haloalkylthio radical bonded via an alkyl group.

“Arylalkyl” represents an aryl radical bonded via an alkyl group, “heteroarylalkyl” denotes a heteroaryl radical bonded via an alkyl group, and “heterocyclylalkyl” denotes a heterocyclyl radical bonded via an alkyl group.

“Cycloalkylalkyl” represents a cycloalkyl radical bonded via an alkyl group, for example (but not limited thereto) cyclopropylmethyl, cyclobutylmethyl, cyclopentylmethyl, cyclohexylmethyl, 1-cyclopropyleth-1-yl, 2-cyclopropyleth-1-yl, 1-cyclopropylprop-1-yl, 3-cyclopropylprop-1-yl.

According to the invention, “haloalkylthio”—on its own or as constituent part of a chemical group—represents straight-chain or branched S-haloalkyl, preferably having 1 to 8, or having 1 to 6 carbon atoms, such as (C₁-C₈)—, (C₁-C₆)—or (C₁-C₄)-haloalkylthio, for example (but not limited thereto) trifluoromethylthio, pentafluoroethylthio, difluoromethyl, 2,2-difluoroeth-1-ylthio, 2,2,2-difluoroeth-1-ylthio, 3,3,3-prop-1-ylthio.

“Halocycloalkyl” and “halocycloalkenyl” denote cycloalkyl or cycloalkenyl, respectively, which are partly or fully substituted by identical or different halogen atoms, such as F, C₁ and Br, or by haloalkyl, such as trifluoromethyl or difluoromethyl, for example 1-fluorocycloprop-1-yl, 2-fluorocycloprop-1-yl, 2,2-difluorocycloprop-1-yl, 1-fluorocyclobut-1-yl, 1-trifluoromethylcycloprop-1-yl, 2-trifluoromethylcycloprop-1-yl, 1-chlorocycloprop-1-yl, 2-chlorocycloprop-1-yl, 2,2-dichlorocycloprop-1-yl, 3,3-difluorocyclobutyl.

According to the invention, “trialkylsilyl”—on its own or as constituent part of a chemical group—represents straight-chain or branched Si-alkyl, preferably having 1 to 8, or having 1 to 6 carbon atoms, such as tri[(C₁-C₈)—, (C₁-C₆)—or (C₁-C₄)-alkyl]silyl, for example (but not limited thereto) trimethylsilyl, triethylsilyl, tri(n-propyl)silyl, tri(isopropyl)silyl, tri(n-butyl)silyl, tri(1-methylprop-1-yl)silyl, tri(2-methylprop-1-yl)silyl, tri(1,1-dimethyleth-1-yl)silyl, tri(2,2-dimethyleth-1-yl)silyl.

If the compounds can form, through a hydrogen shift, tautomers whose structure would not formally be covered by the general formula (I), these tautomers are nevertheless encompassed by the definition of the inventive compounds of the general formula (I), unless a particular tautomer is under consideration. For example, many carbonyl compounds may be present both in the keto form and in the enol form, both forms being encompassed by the definition of the compound of the general formula (I).

According to the nature of the substituents and the way in which they are joined, the compounds of the general formula (I) may take the form of stereoisomers. The possible stereoisomers defined by the specific three-dimensional form thereof, such as enantiomers, diastereomers, Z and E isomers, are all encompassed by the general formula (I). If, for example, one or more alkenyl groups are present, diastereomers (Z and E isomers) may occur. If, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or else on the preparative scale to produce test specimens for biological testing. It is likewise possible to selectively prepare stereoisomers by using stereoselective reactions with use of optically active starting materials and/or auxiliaries. The invention thus also relates to all stereoisomers which are embraced by the general formula (I) but are not shown in their specific stereomeric form, and to mixtures thereof.

If the compounds are obtained as solids, the purification can also be carried out by recrystallization or digestion. If individual compounds (I) cannot be obtained in a satisfactory manner by the routes described below, they can be prepared by derivatization of other compounds (I).

Suitable isolation methods, purification methods and methods for separating stereoisomers of compounds of the general formula (I) are methods generally known to the person skilled in the art from analogous cases, for example by physical processes such as crystallization, chromatographic methods, in particular column chromatography and HPLC (high pressure liquid chromatography), distillation, optionally under reduced pressure, extraction and other methods, any mixtures that remain can generally be separated by chromatographic separation, for example on chiral solid phases. Suitable for preparative amounts or on an industrial scale are processes such as crystallization, for example of diastereomeric salts which can be obtained from the diastereomer mixtures using optically active acids and, if appropriate, provided that acidic groups are present, using optically active bases.

Synthesis of substituted pyrazoles of the general formula (I).

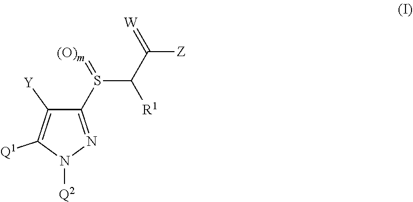

The inventive substituted pyrazoles of the general formula (I) can be prepared proceeding from known processes. The synthesis routes employed and investigated start with commercially available or easily preparable amines, with appropriately substituted aldehydes and with commercially available chemicals such as malonic acid derivatives and nitromethane. In the schemes which follow, the moieties Q¹, Q², Y, W, R′, R², R³, R⁴, R⁵, R⁶, R⁷, R⁸, R⁹, m, n and o of the general formula (I) have the definitions given above, unless illustrative but non-limiting definitions are given.

The inventive compounds of the general formula (Ia) are synthesized via an amide coupling of an acid of the general formula (II) with an amine of the general formula (III) in the presence of an amide coupling reagent, for example T3P, dicyclohexylcarbodiimide, N-(3-dimethylaminopropyl)-N′-ethylcarbodiimide, N,N′-carbonyldiimidazole, 2-chloro-1,3-dimethylimidazolium chloride or 2-chloro-1-methylpyridinium iodide (see Chemistry of Peptide Synthesis, Ed. N. Leo Benoiton, Taylor & Francis, 2006, ISBN-10: 1-57444-454-9). Polymer-supported reagents, for example polymer-supported dicyclohexylcarbodiimide, are also suitable for this coupling reaction. The reaction takes place preferably within the temperature range between 0° C. and 80° C., in an appropriate solvent, for example dichloromethane, acetonitrile, N,N-dimethylformamide or ethyl acetate, and in the presence of a base, for example triethylamine, N,N-diisopropylethylamine or 1,8-diazabicyclo[5.4.0]undec-7-ene (see Scheme 1). For T3P peptide coupling conditions see Organic Process Research & Development 2009, 13, 900-906.

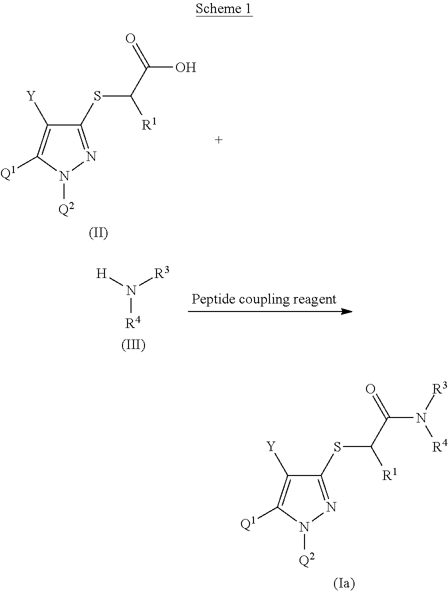

The acid of the general formula (II) can be synthesized by hydrolysis of the compound of the general formula (IV) by or analogously to methods known to the person skilled in the art (Scheme 2). The hydrolysis can be carried out in the presence of a base or a Lewis acid. The base may be a hydroxide salt of an alkali metal (for example lithium, sodium or potassium), and the hydrolysis reaction preferably takes place within the temperature range between room temperature and 120° C.

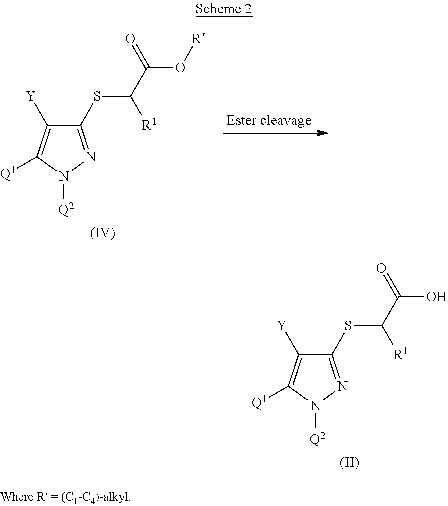

The synthesis of the compound of the general formula (IV) can be prepared by alkylation of the compound of the general formula (V) using a halide of the general formula (VI) in the presence of a base, by or analogously to methods known to the person skilled in the art (see Scheme 3). The base may be a carbonate salt of an alkali metal (for example lithium, sodium, potassium or caesium), and the hydrolysis reaction preferably takes place within the temperature range between room temperature and 150° C. in an appropriate solvent, for example dichloromethane, acetonitrile, N,N-dimethylformamide or ethyl acetate. See J. Med. Chem. 2011, 54(16), 5820-5835 and WO2010/010154.

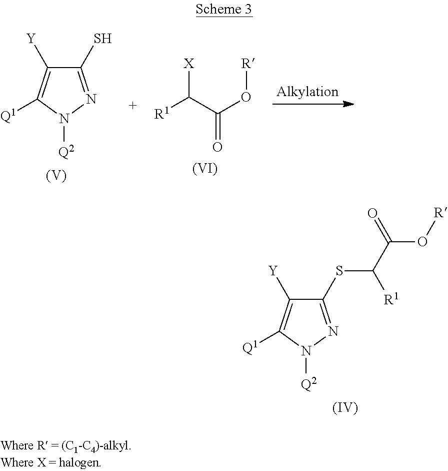

Scheme 4 describes the synthesis of the compound of the general formula (V) by reaction of a pyrazolone of the general formula (VII) in the presence of a sulfurizing reagent, for example phosphorus pentasulfide or Lawesson's reagent in an appropriate solvent, for example toluene.

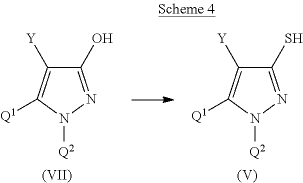

Scheme 5 describes the synthesis of the compound of the general formula (VII) by reaction of a pyrazole of the general formula (VIII) with a halosuccinimide of the general formula (IX) in an appropriate solvent, for example DMF.

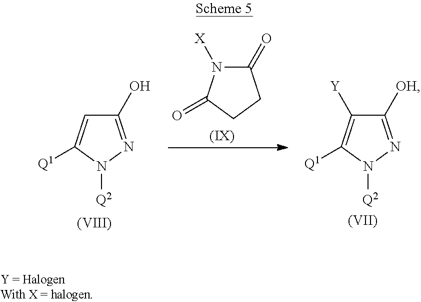

The 3-hydroxypyrazoles (VIII) can be prepared analogously to methods known from the literature from substituted 3-azinylpropynoic acid derivatives and phenyl hydrazines (Scheme 8; e.g.: Adv. Synth. Catal. 2014, 356, 3135-3147) or from substituted azinylacrylic acid derivatives and phenyl hydrazines (Scheme 8; e.g.: J. Heterocyclic Chem., 49, 130 (2012)).

The compounds of the general formula (X) are synthesized via an amide coupling of an acid of the general formula (XII) with an aryl hydrazine or hetaryl hydrazine of the general formula (XI) in the presence of an amide coupling reagent, for example T3P, dicyclohexylcarbodiimide, N-(3-dimethylaminopropyl)-N′-ethylcarbodiimide, N,N′-carbonyldiimidazole, 2-chloro-1,3-dimethylimidazolium chloride or 2-chloro-1-methylpyridinium iodide (see Chemistry of Peptide Synthesis, Ed. N. Leo Benoiton, Taylor & Francis, 2006, ISBN-10: 1-57444-454-9). Polymer-bound reagents, for example polymer-bound dicyclohexylcarbodiimide, are also suitable for this coupling reaction. The reaction takes place preferably within the temperature range between 0° C. and 80° C., in an appropriate solvent, for example dichloromethane, tetrahydrofuran, acetonitrile, N,N-dimethylformamide or ethyl acetate, and in the presence of a base, for example triethylamine, N,N-diisopropylethylamine or 1,8-diazabicyclo[5.4.0]undec-7-ene (see Scheme 8). For T3P peptide coupling conditions see Organic Process Research & Development 2009, 13, 900-906.

The synthesis of the 3-hydroxypyrazoles of the general formula (VIII) takes place by reaction of the compounds of the general formula (X) in the presence of a copper halide such as copper(I) iodide, copper(I) bromide or an acid such as methanesulfonic acid. The reaction preferably takes place in the temperature range between 0° C. and 120° C., in an appropriate solvent, for example 1,2-dichloroethane, acetonitrile, N,N-dimethylformamide, n-propanol or ethyl acetate. Preferably, the reaction takes place in N,N-dimethylformamide.

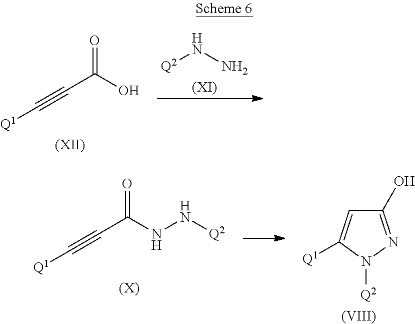

Alternatively, compounds of the general formula (VIII) can be synthesized via an amide coupling of an acid of the general formula (XIV) with an aryl hydrazine or hetaryl hydrazine of the general formula (XI) in the presence of an amide coupling reagent, for example T3P, dicyclohexylcarbodiimide, N-(3-dimethylaminopropyl)-N′-ethylcarbodiimide, N, N′-carbonyldiimidazole, 2-chloro-1,3-dimethylimidazolium chloride or 2-chloro-1-methylpyridinium iodide. The reaction takes place preferably within the temperature range between 0° C. and 80° C., in an appropriate solvent, for example dichloromethane, acetonitrile, N,N-dimethylformamide or ethyl acetate, and in the presence of a base, for example triethylamine, N,N-diisopropylethylamine or 1,8-diazabicyclo[5.4.0]undec-7-ene (see Scheme 7).

The 3-hydroxypyrazoles of the general formula (VIII) are synthesized by reaction of the compounds of the general formula (XIII) in the presence of an iron halide, for example iron(III) chloride. The reaction preferably takes place in the temperature range between 0° C. and 120° C. in an appropriate solvent such as 1,2-dichloroethane, acetonitrile, N,N-dimethylformamide or ethyl acetate.

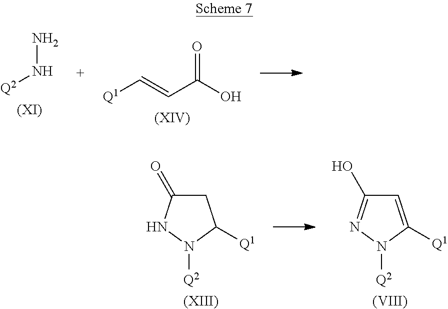

Compounds of the general formula (XVI) can be synthesized by N-arylation of a 3-hydroxypyrazole of the general formula (XVIII) with an aryl halide of the general formula (XVII) in the presence of a copper halide, for example copper(I) iodide. The reaction takes place preferably within the temperature range between 0° C. and 120° C., in an appropriate solvent, for example acetonitrile or N,N-dimethylformamide, and in the presence of a base, for example triethylamine or caesium carbonate (see Scheme 8). The compounds of the general formula (XVIII) can be prepared to methods analogously known to the person skilled in the art (Chem. Med. Chem. 2015, 10, 1184-1199).

The 5-iodopyrazoles of the general formula (XV) are synthesized by reaction of the compounds of the general formula (XVI) in the presence of a base such as lithium diisopropylamide and iodine. The reaction (Scheme 8) preferably takes place in the temperature range between −78° C. and −60° C. in an appropriate solvent such as diethyl ether and tetrahydrofuran.

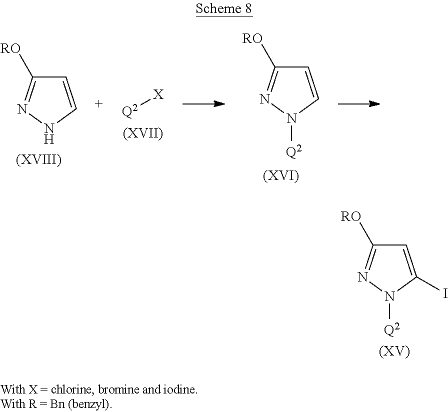

A compound of the general formula (XIX) can be prepared by reaction of a compound of the formula (XV) in a suitable solvent with a Q¹-M (XX) with addition of an appropriate amount of a transition metal catalyst, in particular palladium catalysts such as palladium diacetate or bis(triphenylphosphine)palladium(II) dichloride or nickel catalysts, for example nickel(II) acetylacetonate or bis(triphenylphosphine)nickel(II) chloride, preferably at elevated temperature in an organic solvent such as 1,2-dimethoxyethane (Scheme 9). The “M” radical represents, for example, Mg—X, Zn—X, Sn((C₁-C₄)-alkyl)₃, lithium, copper or B(OR^{b})(OR^{c}), where the R^{b} and R^{c} radicals are independently, for example, hydrogen, (C₁-C₄)-alkyl, or, if the radicals R^{b} and R^{c} are bonded to one another, together are ethylene or propylene. Generally suitable are cross-coupling methods described in R. D. Larsen, Organometallics in Process Chemistry 2004 Springer Verlag, in I. Tsuji, Palladium Reagents and Catalysts 2004 Wiley, and in M. Beller, C. Bolm, Transition Metals for Organic Synthesis 2004 VCH-Wiley. Further suitable synthesis methods are described in Chem. Rev. 2006, 106, 2651; Platinum Metals Review, 2009, 53, 183; Platinum Metals Review 2008, 52, 172 and Acc. Chem. Res. 2008, 41, 1486.

The 3-hydroxypyrazoles of the general formula (VIII) can be synthesized by reaction of a compound of the general formula (XIX) in the presence of a Brønsted acid, for example trifluoromethanesulfonic acid. The reaction preferably takes place in the temperature range between 0° C. and 120° C., in an appropriate solvent, for example dichloromethane (Scheme 9).

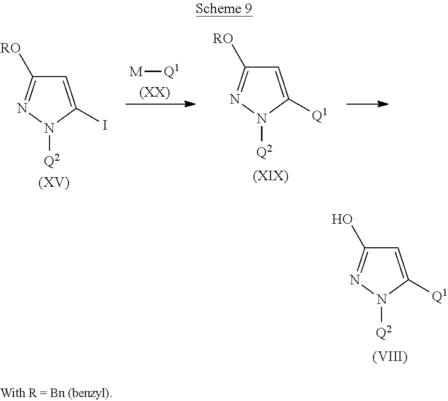

The compound of the general formula (XXII) can be prepared by alkylation of the compound of the general formula (XXIII) with a halide of the general formula (VI) in the presence of a base, by or analogously to methods known to the person skilled in the art (see Scheme 10). The base may be a carbonate salt of an alkali metal (for example lithium, sodium, potassium or caesium), and the reaction preferably takes place within the temperature range between room temperature and 150° C. in an appropriate solvent, for example dichloromethane, acetonitrile, N,N-dimethylformamide or ethyl acetate.

Compounds of the general formula (XXI) can be prepared by diazotization or Sandmeyer reaction with the compound of the general formula (XXII) using the customary organic and inorganic nitrites such as 1,1-dimethylethyl nitrite, tert-butyl nitrite or isoamyl nitrite in the presence of usable reagents such as mixtures of copper(I) and copper(II) bromide/chloride or iodine (Scheme 10). The reaction preferably takes place in the temperature range between room temperature and 0° C. and 120° C. in an appropriate solvent such as dichloromethane, acetonitrile, N,N-dimethylformamide or diiodomethane. Scheme 10 describes the synthesis of the compound of the general formula (XXI) by reaction of a pyrazole of the general formula (XXII) with a halosuccinimide of the general formula (IX) in an appropriate solvent, for example DMF.

A compound of the general formula (IV) can be prepared by reaction of a compound of the formula (XXI) in a suitable solvent with a Q¹-M (XX) with addition of an appropriate amount of a transition metal catalyst, in particular palladium catalysts such as palladium diacetate or bis(triphenylphosphine)palladium(II) dichloride or nickel catalysts, for example nickel(II) acetylacetonate or bis(triphenylphosphine)nickel(II) chloride, preferably at elevated temperature in an organic solvent such as 1,2-dimethoxyethane (Scheme 9). The “M” radical represents, for example, Mg—X, Zn—X, Sn((C₁-C₄)-alkyl)₃, lithium, copper or B(OR^{b})(OR^{c}), where the R^{b} and R^{c} radicals are independently, for example, hydrogen, (C₁-C₄)-alkyl, or, if the radicals R^{b} and R^{c} are bonded to one another, together are ethylene or propylene. Generally suitable are cross-coupling methods described in R. D. Larsen, Organometallics in Process Chemistry 2004 Springer Verlag, in I. Tsuji, Palladium Reagents and Catalysts 2004 Wiley, and in M. Beller, C. Bolm, Transition Metals for Organic Synthesis 2004 VCH-Wiley. Further suitable synthesis methods are described in Chem. Rev. 2006, 106, 2651; Platinum Metals Review, 2009, 53, 183; Platinum Metals Review 2008, 52, 172 and Acc. Chem. Res. 2008, 41, 1486.

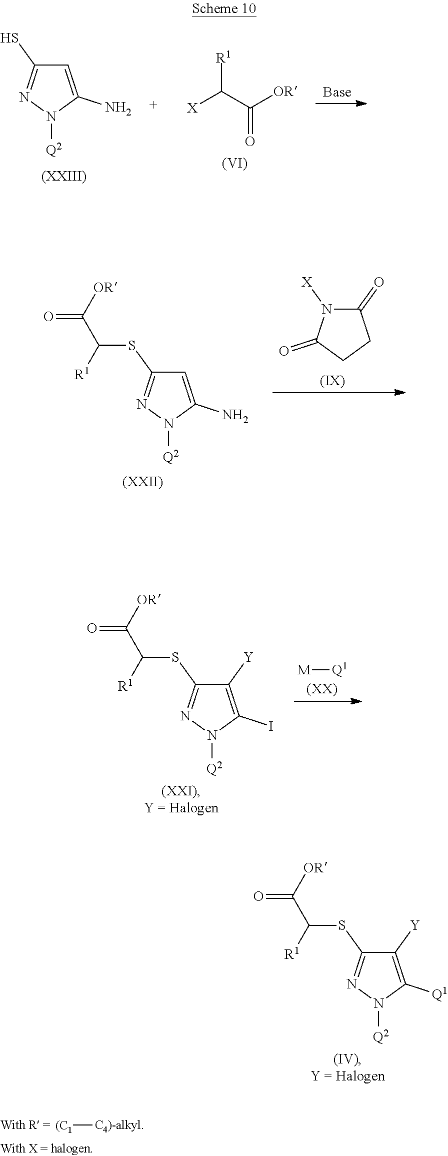

Scheme 11 describes the synthesis of the compound of the general formula (XXIII) by reaction of a pyrazolone of the general formula (XXIV) in the presence of a sulfurizing reagent, for example phosphorus pentasulfide or Lawesson's reagent in an appropriate solvent, for example toluene. The compounds of the general formula (XXIV) are commercially available or can be prepared analogously to methods known to the person skilled in the art.

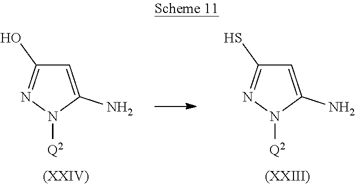

The compound of the general formula (IV) can be prepared by reaction of a 3-aminopyrazole of the general formula (XXVI) with a disulfide of the general formula (XXV) in the presence of an organic nitrite, for example 1,1-dimethylethyl nitrite, tert-butyl nitrite or isoamyl nitrite, in the presence of a metal, for example copper (see Scheme 12). The reaction preferably takes place within the temperature range between room temperature and 120° C. in an appropriate solvent, for example dichloromethane, acetonitrile, N,N-dimethylformamide or 1,2-dichloroethane.

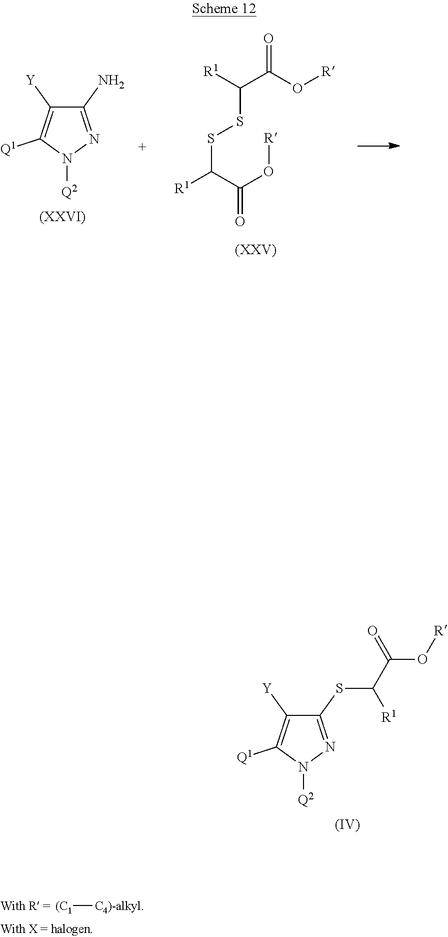

The acid of the general formula (XXVI) can be synthesized by reaction of the compound of the general formula (XXVII) in the presence of a Lewis acid, for example trifluoroacetic acid, by or analogously to methods known to the person skilled in the art (Scheme 13). The reaction preferably takes place within the temperature range between room temperature and 140° C.

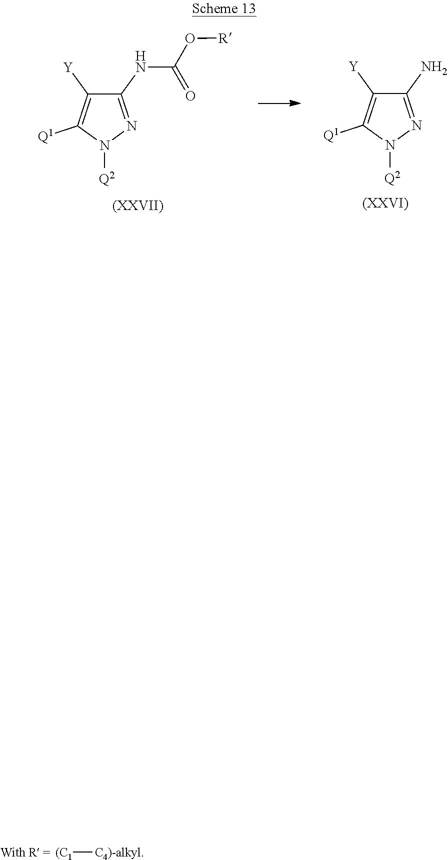

The compounds of the general formula (XXVII) are synthesized via a Curtius reaction of an acid of the general formula (XXIX) with an azide of the general formula (XXVIII). The reaction takes place preferably within the temperature range between 0° C. and 100° C., in an appropriate solvent, for example tert-butanol, and in the presence of a base, for example triethylamine, N,N-diisopropylethylamine or 1,8-diazabicyclo[5.4.0]undec-7-ene (see Scheme 14).

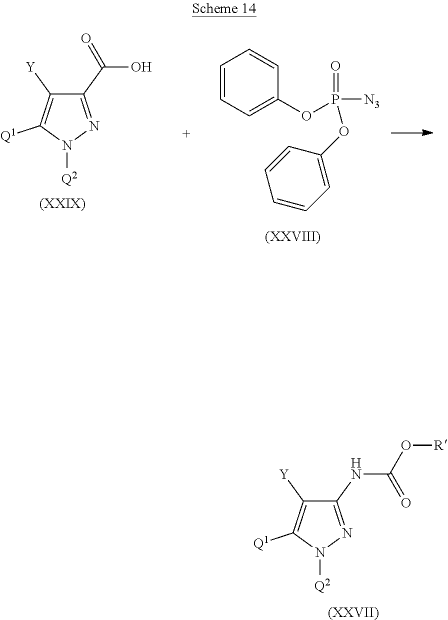

The acid of the general formula (XXIX) can be synthesized by hydrolysis of the compound of the general formula (XXX) by or analogously to methods known to the person skilled in the art (Scheme 15). The hydrolysis can be carried out in the presence of a base or a Lewis acid. The base may be a hydroxide salt of an alkali metal (for example lithium, sodium or potassium), and the hydrolysis reaction preferably takes place within the temperature range between room temperature and 120° C.

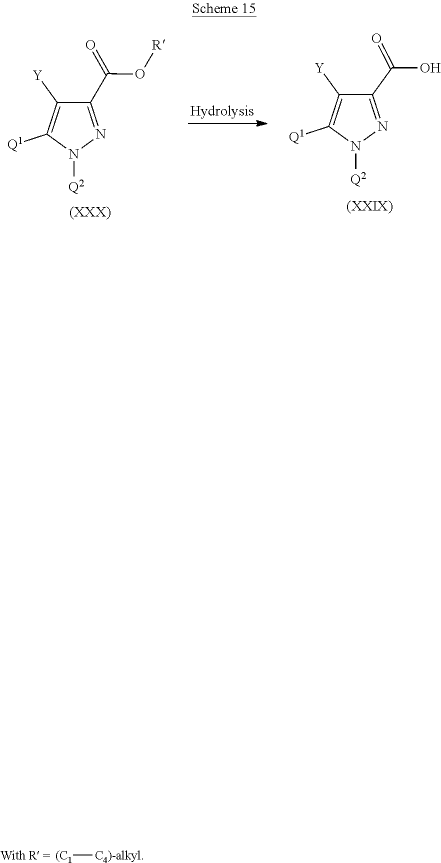

Scheme 16 describes the synthesis of the compound of the general formula (XXX) by reaction of a pyrazole of the general formula (XXXI) with a halosuccinimide of the general formula (IX) in an appropriate solvent, for example DMF.

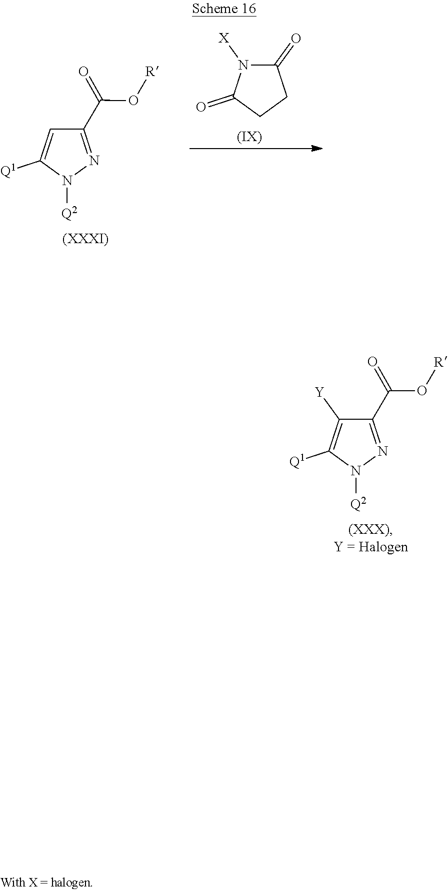

The compounds of the general formula (XXXI) are synthesized via a condensation of a diketo ester of the general formula (XXXIII) with an aryl hydrazine or hetaryl hydrazine of the general formula (XI) in the presence of a Brønsted acid, for example acetic acid or hydrogen chloride, in an appropriate solvent, for example methanol, ethanol, isopropanol, n-butanol, tetrahydrofuran, dioxane, toluene or chlorobenzene (Scheme 17). The reaction preferably takes place in the temperature range between 0° C. and 150° C. The compounds of the general formulas (XI) and (XXXIII) are commercially available or can be prepared analogously to methods known to the person skilled in the art.

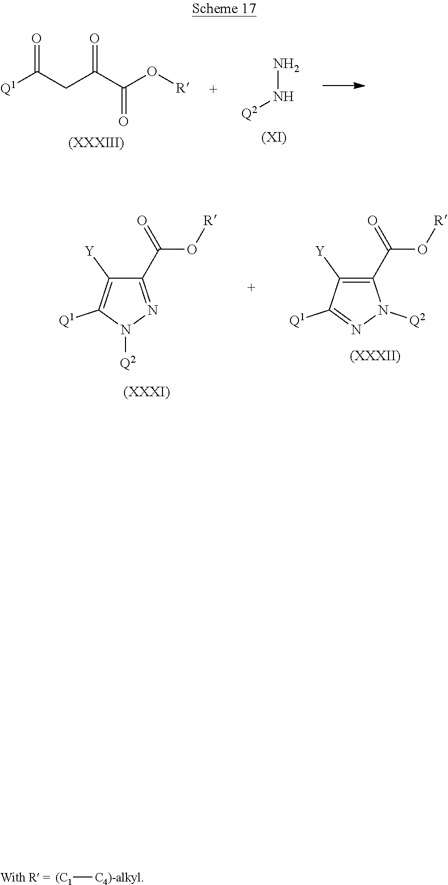

A compound of the general formula (XXXIV) can be prepared, for example, by reaction of a compound of the formula (VII) in a suitable solvent with a metal cyanide M-CN(XXXV) with addition of an appropriate amount of a transition metal catalyst, in particular palladium catalysts such as palladium(O)tetrakis(triphenylphosphine) or palladium diacetate or bis(triphenylphosphine)palladium(II) dichloride or nickel catalysts such as nickel(II) acetylacetonate or bis(triphenylphosphine)nickel(II) chloride, preferably at elevated temperature in an organic solvent, for example 1,2-dimethoxyethane or N,N-dimethylformamide (Scheme 18). The “M” radical represents, for example, magnesium, zinc, lithium or sodium. Cross-coupling methods that are suitable in general are those described in R. D. Larsen, Organometallics in Process Chemistry 2004 Springer Verlag, in I. Tsuji, Palladium Reagents and Catalysts 2004 Wiley, and in M. Beller, C. Bolm, Transition Metals for Organic Synthesis 2004 VCH-Wiley. Further suitable synthesis methods are described in Chem. Rev. 2006, 106, 2651; Platinum Metals Review, 2009, 53, 183; Platinum Metals Review 2008, 52, 172 and Acc. Chem. Res. 2008, 41, 1486.

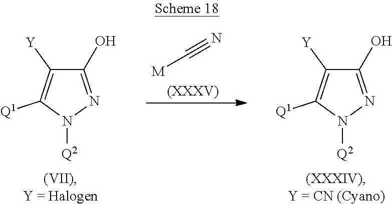

Compounds of the general formulae (XXXVI) and (XXXVII) can be prepared by reaction of a compound of the formula (IV) in the presence of an oxidizing agent, for example mCPBA (3-chloroperbenzoic acid), in an appropriate solvent, for example dichloromethane or 1,2-dichloroethane (Scheme 19). The reaction preferably takes place in the temperature range between −30° C. and 100° C.

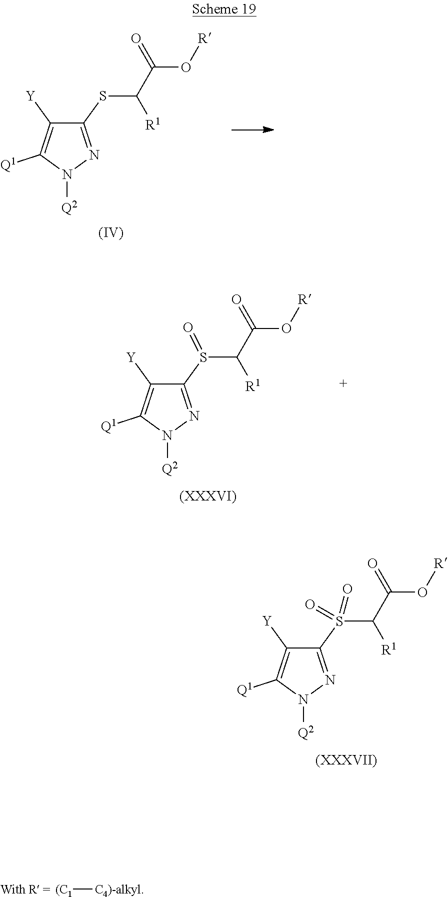

Scheme 20 describes the synthesis of the compound of the general formula (IV) by reaction of a pyrazole of the general formula (XXXVIII) with a halosuccinimide of the general formula (IX) in an appropriate solvent, for example DMF.

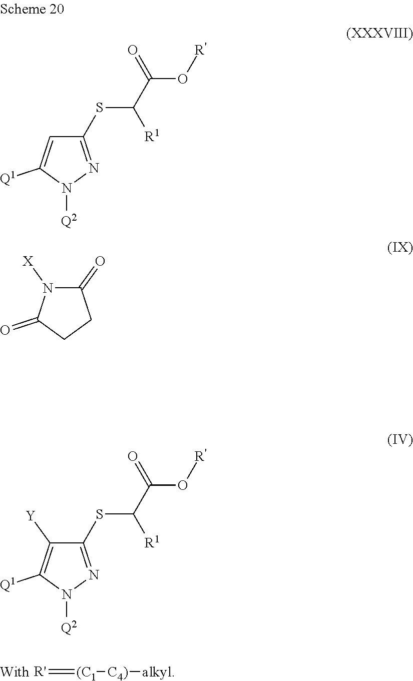

Selected detailed synthesis examples for the inventive compounds of the general formula (I) are adduced below. The example numbers mentioned correspond to the numbering scheme in Tables I.1 to I.49 below. The ¹H NMR, ¹³C-NMR and ¹⁹F-NMR spectroscopy data reported for the chemical examples described in the sections which follow (400 MHz for ¹H NMR and 150 MHz for ¹³C-NMR and 375 MHz for ¹⁹F-NMR, solvent CDCl₃, CD₃OD or d₆-DMSO, internal standard: tetramethylsilane δ=0.00 ppm) were obtained on a Bruker instrument, and the signals listed have the meanings given below: br=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets. In the case of diastereomer mixtures, either the significant signals for each of the two diastereomers are reported or the characteristic signal of the main diastereomer is reported. The abbreviations used for chemical groups have, for example, the following meanings: Me=CH₃, Et=CH₂CH₃, t-Hex=C(CH₃)₂CH(CH₃)₂, t-Bu=C(CH₃)₃, n-Bu=unbranched butyl, n-Pr=unbranched propyl, i-Pr=branched propyl, c-Pr=cyclopropyl, c-Hex=cyclohexyl.

### SYNTHESIS EXAMPLES

### Synthesis Example No. 1-005

Synthesis stage 1: Ethyl(3Z)-4-(3,4-difluorophenyl)-4-hydroxy-2-oxobut-3-enoate

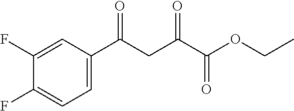 [SMILES: CCOC(=O)C(=O)CC(=O)c1ccc(F)c(F)c1]

A 1 M lithium trimethyl-N-(trimethylsilyl)silanaminide solution in THF (33.63 ml, 33.63 mmol, 1.05 equiv) was dissolved in diethyl ether (80 ml) under nitrogen atmosphere and cooled to −78° C. with a dry ice bath. Added dropwise to the solution within 10 min was a solution of 1-(3,4-difluorophenyl)ethanone (5.0 g, 35.02 mmol, 1.0 equiv.) in diethyl ether (20 ml). The resulting reaction mixture was stirred at −78° C. for 3 h and then at room temperature overnight. Subsequently, the suspension was cooled down to 0 to 4° C. with an ice bath, 1 M hydrochloric acid was added thereto and the mixture was stirred at room temperature for 30 min. The reaction mixture was extracted three times with 100 ml each time of ethyl acetate. The organic phase was dried over magnesium sulfate and the solvent was removed under reduced pressure. The desired ethyl(3Z)-4-(3,4-difluorophenyl)-4-hydroxy-2-oxobut enoate product was isolated in the form of a white solid (8.00 g, 97% of theory). ¹H-NMR (400 MHz, CDCl₃δ, ppm) 15.05 (bs, 1H), 7.87-7.77 (m, 2H), 7.33-7.29 (m, 1H), 7.00 (s, 1H), 4.41 (q, 2H), 1.42 (t, 3H).

Synthesis stage 2: Ethyl 5-(3,4-difluorophenyl)-1-(2-fluorophenyl)-1H-pyrazole-3-carboxylate

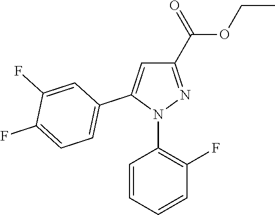 [SMILES: CCOC(=O)c1cc(-c2ccc(F)c(F)c2)n(-c2ccccc2F)n1]

Ethyl(3Z)-4-(3,4-difluorophenyl)-4-hydroxy-2-oxobut-3-enoate (2.0 g, 7.81 mmol, 1.0 equiv) and (2-fluorophenyl)hydrazine hydrochloride (1:1) (1.90 g, 11.71 mmol, 1.5 equiv) were suspended in 1.25 M hydrogen chloride in ethanol (30 ml) and heated to boiling for 4 h. The reaction mixture was neutralized with a saturated sodium hydrogencarbonate solution and then extracted three times with 50 ml each time of dichloromethane. The organic phase was dried over magnesium sulfate and the solvent was removed under reduced pressure. By final purification of the resulting crude product by column chromatography (ethyl acetate/heptane gradient), ethyl 5-(3,4-difluorophenyl)-1-(2-fluorophenyl)-1H-pyrazole-3-carboxylate was isolated in the form of a yellow oil (3.02 g, 98% of theory). ¹H-NMR (400 MHz, CDCl_{3 δ, ppm)} 7.57-7.53 (m, 1H), 7.47-7.41 (m, 1H), 7.29-7.25 (m, 1H), 7.13-7.01 (m, 4H), 6.97-6.93 (m, 1H), 4.46 (q, 2H), 1.43 (t, 3H).

Synthesis stage 3: Ethyl 4-bromo-5-(3,4-difluorophenyl)-1-(2-fluorophenyl)-1H-pyrazole-3-carboxylate

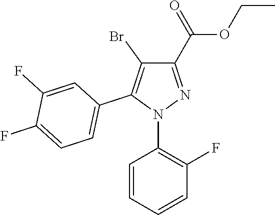 [SMILES: CCOC(=O)c1nn(-c2ccccc2F)c(-c2ccc(F)c(F)c2)c1Br]

Ethyl 5-(3,4-difluorophenyl)-1-(2-fluorophenyl)-1H-pyrazole-3-carboxylate (2.38 g, 6.87 mmol, 1.0 equiv) was dissolved in acetic acid (20 ml), and bromine (1.10 g, 6.87 mmol, 1.0 equiv) was added at room temperature. The resulting reaction mixture was stirred at room temperature for 2 h, and then more bromine (0.55 g, 3.43 mmol, 0.5 equiv) was added. The reaction was stirred at room temperature overnight and then ice-water (150 ml) was added, which resulted in a precipitate. The precipitate was dissolved in dichloromethane (100 ml) and extracted with saturated sodium thiosulfate solution. The organic phase was dried over magnesium sulfate and the solvent was removed under reduced pressure. By final purification of the resulting crude product by column chromatography (ethyl acetate/heptane gradient), ethyl 4-bromo-5-(3,4-difluorophenyl)-1-(2-fluorophenyl)-1H-pyrazole-3-carboxylate was isolated in the form of a white solid (2.4 g, 78% of theory). ¹H-NMR (400 MHz, CDCl₃δ, ppm) 7.51-7.45 (m, 1H), 7.44-7.39 (m, 1H), 7.27-7.22 (m, 1H), 7.17-6.98 (m, 4H), 4.49 (q, 2H), 1.44 (t, 3H).

Synthesis stage 4:4-Bromo-5-(3,4-difluorophenyl)-1-(2-fluorophenyl)-1H-pyrazole-3-carboxylic acid

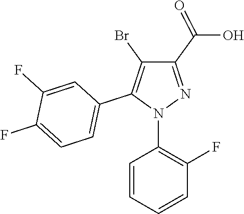 [SMILES: O=C(O)c1nn(-c2ccccc2F)c(-c2ccc(F)c(F)c2)c1Br]

Ethyl 4-bromo-5-(3,4-difluorophenyl)-1-(2-fluorophenyl)-1H-pyrazole-3-carboxylate (2.08 g, 4.88 mmol, 1.0 equiv) and lithium hydroxide (350.6 mg, 14.64 mmol, 3.0 equiv) were dissolved in a mixture of THF and water (18 ml, THF:water=7:2) and heated to boiling for 6 h. After cooling, the solution was acidified with 2 M hydrochloric acid and extracted twice with ethyl acetate (50 ml). The organic phase was dried over magnesium sulfate and the solvent was removed under reduced pressure. 4-Bromo-5-(3,4-difluorophenyl)-1-(2-fluorophenyl)-1H-pyrazole-3-carboxylic acid was isolated in the form of a white solid (1.51 g, 77% of theory). ¹H-NMR (400 MHz, CDCl₃δ, ppm) 7.52-7.42 (m, 1H), 7.29-7.26 (m, 1H), 7.48 (m, 1H), 7.19-7.08 (m, 3H), 7.03-7.00 (m, 1H).

Synthesis stage 5: tert-Butyl[4-bromo-5-(3,4-difluorophenyl)-1-(2-fluorophenyl)-1H-pyrazol-3-yl]carbamate

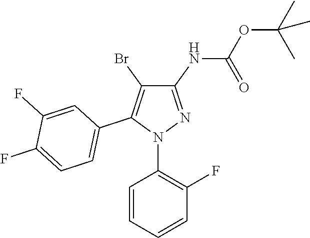 [SMILES: CC(C)(C)OC(=O)Nc1nn(-c2ccccc2F)c(-c2ccc(F)c(F)c2)c1Br]

4-Bromo-5-(3,4-difluorophenyl)-1-(2-fluorophenyl)-1H-pyrazole-3-carboxylic acid (1.02 g, 2.57 mmol, 1.0 equiv) and triethylamine (0.90 ml, 6.42 mmol, 2.5 equiv) were dissolved in tert-butanol (15 ml), and diphenylphosphorus azide (0.55 ml, 2.57 mmol, 1.0 equiv) was added at room temperature. The reaction was stirred at 60° C. for 4 h, in the course of which evolution of gas occurred. After cooling to room temperature, a saturated sodium hydrogencarbonate solution (10 ml) was added to the reaction solution, and the mixture was extracted twice with ethyl acetate (50 ml). The organic phase was dried over magnesium sulfate and the solvent was removed under reduced pressure. By final purification of the resulting crude product by column chromatography (ethyl acetate/heptane gradient), tert-butyl[4-bromo-5-(3,4-difluorophenyl)-1-(2-fluorophenyl)-1H-pyrazol-3-yl]carbamate was isolated in the form of a white solid (625 mg, 51% of theory). ¹H-NMR (400 MHz, CDCl₃δ, ppm) 7.52-7.48 (m, 1H), 7.38-7.32 (m, 1H), 7.21 (dt, 1H), 7.15-7.09 (m, 2H), 7.05-6.97 (m, 2H), 6.48 (bs, 1H), 1.56 (s, 9H).

Synthesis stage 6:4-Bromo-5-(3,4-difluorophenyl)-1-(2-fluorophenyl)-1H-pyrazole-3-amine

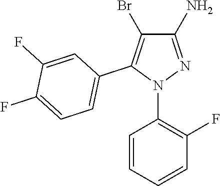 [SMILES: Nc1nn(-c2ccccc2F)c(-c2ccc(F)c(F)c2)c1Br]

tert-Butyl[4-bromo-5-(3,4-difluorophenyl)-1-(2-fluorophenyl)-1H-pyrazol-3-yl]carbamate (625 mg, 1.34 mmol, 1.0 equiv) and trifluoroacetic acid (0.21 ml, 2.67 mmol, 2.0 equiv) were dissolved in dichloromethane (20 ml) and heated to boiling for 2 h. After cooling, the reaction mixture was diluted with saturated sodium hydrogencarbonate solution (10 ml), and then extracted twice with DCM (50 ml). 4-Bromo-5-(3,4-difluorophenyl)-1-(2-fluorophenyl)-1H-pyrazole-3-amine was isolated in the form of a white solid (480 mg, 99% of theory). ¹H-NMR (400 MHz, CDCl₃δ, ppm) 7.96 (bs, 2H), 7.46-7.36 (m, 2H), 7.26-7.23 (m, 1H), 7.18-7.10 (m, 3H), 7.08-7.00 (m, 1H).

Synthesis stage 7: Ethyl {[4-bromo-5-(3,4-difluorophenyl)-1-(2-fluorophenyl)-1H-pyrazol-3-yl]sulfanyl}acetate (Example No. 1-002)

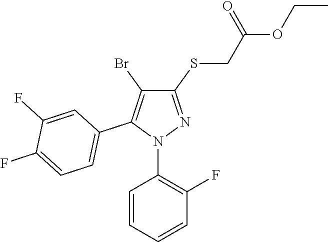 [SMILES: CCOC(=O)CSc1nn(-c2ccccc2F)c(-c2ccc(F)c(F)c2)c1Br]

4-Bromo-5-(3,4-difluorophenyl)-1-(2-fluorophenyl)-1H-pyrazole-3-amine (223 mg, 0.61 mmol, 1.0 equiv), diethyl 2,2′-disulfanediyldiacetate (0.22 ml, 1.21 mmol, 2 equiv) and copper powder (7.70 mg, 0.12 mmol, 0.2 equiv) were suspended in 1,2-dichloroethane (10 ml) and heated to 60° C. for 15 min. Subsequently, tert-butyl nitrite (0.22 ml, 1.21 mmol, 2.0 equiv) was added dropwise and the reaction mixture was heated to 80° C. for 4 h. After cooling, the reaction mixture was diluted with water (10 ml) and dichloromethane (30 ml), and then extracted twice with DCM (50 ml). The phases were separated with a phase separator. By final purification of the resulting crude product by column chromatography (ethyl acetate/heptane gradient), ethyl {[4-bromo-5-(3,4-difluorophenyl)-1-(2-fluorophenyl)-1H-pyrazol-3-yl]sulfanyl}acetate was isolated in the form of a white solid (129 mg, 44% of theory). ¹H-NMR (400 MHz, CDCl₃δ, ppm) 7.44-7.34 (m, 2H), 7.26-7.20 (m, 1H), 7.13-7.02 (m, 3H), 6.99-6.96 (m, 1H), 4.22 (q, 2H), 3.89 (s, 2H), 1.24 (t, 3H).

Synthesis stage 8: {[4-Bromo-5-(3,4-difluorophenyl)-1-(2-fluorophenyl)-1H-pyrazol-3-yl]sulfanyl}acetic acid (Example No. 1-005)

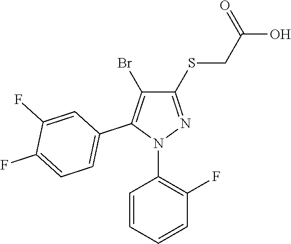 [SMILES: O=C(O)CSc1nn(-c2ccccc2F)c(-c2ccc(F)c(F)c2)c1Br]

Ethyl {[4-bromo-5-(3,4-difluorophenyl)-1-(2-fluorophenyl)-1H-pyrazol-3-yl]sulfanyl}acetate (83 mg, 0.18 mmol, 1.0 equiv) and lithium hydroxide (12.6 mg, 0.53 mmol, 3.0 equiv) were dissolved in a mixture of THF and water (9 ml, THF:water=7:2) and heated to boiling for 6 h. After cooling, the solution was acidified with 2 M hydrochloric acid and extracted twice with ethyl acetate (50 ml). The organic phase was dried over magnesium sulfate and the solvent was removed under reduced pressure. {[4-Bromo-5-(3,4-difluorophenyl)-1-(2-fluorophenyl)-1H-pyrazol-3-yl]sulfanyll acetic acid was isolated in the form of a white solid (56 mg, 68% of theory). ¹H-NMR (400 MHz, CDCl₃δ, ppm) 7.45-7.35 (m, 2H), 7.27-7.22 (m, 1H), 7.18-7.09 (m, 3H), 7.01-6.97 (m, 3H), 3.86 (s, 2H).

### Synthesis Example No. 1-008

Synthesis stage 1: Ethyl {[4-bromo-1-(2,3-difluorophenyl)-5-phenyl-1H-pyrazol-3-yl]sulfonyl}acetate

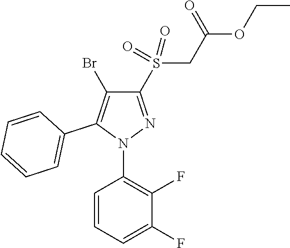 [SMILES: CCOC(=O)CS(=O)(=O)c1nn(-c2cccc(F)c2F)c(-c2ccccc2)c1Br]

Ethyl {[4-bromo-1-(2,3-difluorophenyl)-5-phenyl-1H-pyrazol-3-yl]sulfanyl}acetate (115 mg, 0.25 mmol, 1.0 equiv) was dissolved in dichloromethane (5 ml). 3-Chloroperoxybenzoic acid (114 mg, 0.51 mmol, 2.0 equiv, 77% pure) was added to the solution. The resulting reaction mixture was stirred at room temperature overnight. The reaction mixture was diluted with dichloromethane (30 ml) and extracted with a saturated sodium hydrogencarbonate solution. The organic phase was dried over magnesium sulfate and the solvent was removed under reduced pressure. By final purification of the resulting crude product by column chromatography (ethyl acetate/heptane gradient), ethyl {[4-bromo (2,3-difluorophenyl)-5-phenyl-1H-pyrazol-3-yl]sulfonyllacetate was isolated in the form of a colourless solid (90 mg, 55% of theory, 80% pure). ¹H-NMR (400 MHz, CDCl₃δ, ppm) 7.71-7.63 (m, 2H), 7.58-7.32 (m, 6H), 4.76 (s, 2H), 4.12 (q, 2H), 1.13 (t, 3H).

### Synthesis Example No. II-001

Synthesis stage 1:1H-Pyrazol-3-ol

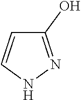

Methyl (2E)-3-methoxyacrylate (20.00 g, 172.24 mmol, 1.0 equiv) was dissolved in methanol (15 ml), and hydrazine hydrate (1:1) (8.38 ml, 172.24 mmol, 1 equiv) was added dropwise, in the course of which the reaction mixture heated up to boiling. Subsequently, the reaction mixture was heated to boiling for 2 h and then concentrated under reduced pressure. The crude product was used in the next synthesis stage without further purification. 1H-Pyrazol-3-ol was isolated in the form of a yellow oil (15.30 g, 95% of theory). ¹H-NMR (400 MHz, CDCl₃δ, ppm) 9.85 (bs, 1H), 7.35 (d, 1H), 5.43 (d, 1H).

Synthesis stage 2:1-(3-Hydroxy-1H-pyrazol-1-yl)ethanone

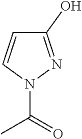

1H-Pyrazol-3-ol (15.30 g, 181.97 mmol, 1.0 equiv) was dissolved in pyridine (100 ml). Added dropwise to the solution within 30 min was a mixture of acetic anhydride (19.00 ml, 201.37 mmol) and pyridine (10 ml). The resulting reaction mixture was heated to a temperature of 100° C. for 2 h. After cooling to room temperature, the solvent was removed under reduced pressure. The residue was stirred with diethyl ether (50 ml), in the course of which it crystallized. 1-(3-Hydroxy-1H-pyrazol-1-yl)ethanone was isolated in the form of a pale yellow solid (18.00 g, 78% of theory). ¹H-NMR (400 MHz, CDCl₃δ, ppm) 10.98 (bs, 1H), 8.12 (d, 1H), 6.01 (d, 2H), 2.47 (s, 3H).

Synthesis stage 3:3-(Benzyloxy)-1H-pyrazole

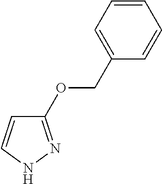 [SMILES: c1ccc(COc2cc[nH]n2)cc1]

1-(3-Hydroxy-1H-pyrazol-1-yl)ethanone (18.00 g, 142.73 mmol, 1.0 equiv), (bromomethyl)benzene (16.98 ml, 142.73 mmol, 1.0 equiv) and potassium carbonate (19.73 g, 142.73 mmol, 1.0 equiv) were suspended in butan-2-one (200 ml) and heated to boiling for 3 h. After cooling, the reaction mixture was filtered and the filtrate was concentrated under reduced pressure. The residue was dissolved in THF (150 ml) and methanol (150 ml), and a 6 M sodium hydroxide solution (24 ml) was added. The reaction mixture was stirred at room temperature for 1 h and then the solvent was concentrated under reduced pressure. The residue was dissolved in dichloromethane (150 ml) and washed twice with water (50 ml). The organic phase was dried over magnesium sulfate and the solvent was removed under reduced pressure. The crude product was used in the next synthesis stage without further purification. 3-(Benzyloxy)-1H-pyrazole was isolated in the form of a yellow oil (19.82 g, 75% of theory). ¹H-NMR (400 MHz, CDCl₃δ, ppm) 10.55 (bs, 1H), 7.43-7.23 (m, 6H), 5.74 (d, 1H), 5.20 (s, 2H).

Synthesis stage 4:3-(Benzyloxy)-1-(2-fluorophenyl)-1H-pyrazole

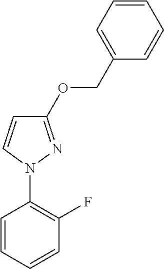 [SMILES: Fc1ccccc1-n1ccc(OCc2ccccc2)n1]

3-(Benzyloxy)-1H-pyrazole (2.74 g, 15.73 mmol, 1.0 equiv), 1-fluoro-2-iodobenzene (1.83 ml, 15.73 mmol, 1 equiv), copper(I) iodide (299 mg, 1.57 mmol, 0.10 equiv), potassium carbonate (5.43 g, 39.31 mmol, 2.50 equiv) and N,N′-dimethylethane-1,2-diamine (0.87 ml, 7.88 mmol, 0.10 equiv) were suspended in toluene (25 ml) and heated to 115° C. for 5 h. The solvent was removed under reduced pressure, and the residue was extracted with dichloromethane (50 ml) and water (20 ml). The organic phase was dried over magnesium sulfate and the solvent was removed under reduced pressure. By final purification of the resulting crude product by column chromatography (ethyl acetate/heptane gradient), 3-(benzyloxy)-1-(2-fluorophenyl)-1H-pyrazole was isolated in the form of a yellow solid (2.09 g, 49% of theory, 80% pure). ¹H-NMR (400 MHz, CDCl₃δ, ppm) 7.91-7.85 (m, 2H), 7.51-7.49 (m, 2H), 7.42-7.32 (m, 3H), 7.25-7.16 (m, 3H), 5.95 (d, 1H), 5.32 (s, 2H).

Synthesis stage 5:3-(Benzyloxy)-1-(2-fluorophenyl)-5-iodo-1H-pyrazole

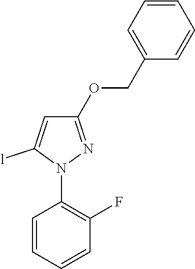

3-(Benzyloxy)-1-(2-fluorophenyl)-1H-pyrazole (4.81 g, 17.92 mmol, 1.0 equiv) was dissolved in THF (140 ml) and cooled to −78° C. with a cold bath (dry ice/acetone). Added dropwise to this cooled solution within 10 min was a 2 M lithium diisopropylamide solution in THF, and the mixture was stirred at this temperature for a further 30 min. This was followed by dropwise addition of iodine (4.55 g, 17.92 mmol, 1 equiv) dissolved in THF (10 ml) within 10 min. Thereafter, the reaction mixture was warmed to room temperature overnight and extracted with water (300 ml) and ethyl acetate (250 ml). The organic phase was washed with a saturated sodium thiosulfate solution (75 ml). Subsequently, the organic phase was dried over magnesium sulfate, and the solvent was removed under reduced pressure. By final purification of the resulting crude product by column chromatography (ethyl acetate/heptane gradient), 3-(benzyloxy)-1-(2-fluorophenyl)-5-iodo-1H-pyrazole was isolated in the form of a brown oil (5.20 g, 73% of theory). ¹H-NMR (400 MHz, CDCl₃δ, ppm) 7.90-7.84 (m, 2H), 7.52-7.47 (m, 2H), 7.41-7.31 (m, 2H), 7.25-7.15 (m, 3H), 5.94 (s, 1H), 5.31 (s, 2H).

Synthesis stage 6:5-[3-(Benzyloxy)-1-(2-fluorophenyl)-1H-pyrazol-5-yl]-2-fluoropyridine

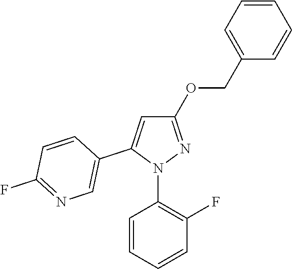 [SMILES: Fc1ccc(-c2cc(OCc3ccccc3)nn2-c2ccccc2F)cn1]

3-(Benzyloxy)-1-(2-fluorophenyl)-5-iodo-1H-pyrazole (2.20 g, 5.58 mmol, 1.0 equiv), (6-fluoropyridin-3-yl)boronic acid (1.18 mg, 8.37 mmol, 1.5 equiv) and caesium carbonate (7.27 mg, 22.32 mmol, 4 equiv) were suspended in a solvent mixture consisting of toluene (56 ml), ethanol (16 ml) and water (8 ml), and degassed and flooded with argon three times in succession. Bis(di-tert-butyl(4-dimethylaminophenyl)phosphino)palladium(II) chloride was added to the suspension. Then the reaction mixture was heated to boiling for 2 h. The solvent was removed under reduced pressure, and the residue was extracted with dichloromethane (750 ml) and water (30 ml). The organic phase was dried over magnesium sulfate and the solvent was removed under reduced pressure. By final purification of the resulting crude product by column chromatography (ethyl acetate/heptane gradient), 5-[3-(benzyloxy) (2-fluorophenyl)-1H-pyrazol-5-yl]-2-fluoropyridine was isolated in the form of a yellow solid (1.01 g, 45% of theory). ¹H-NMR (400 MHz, DMSO-d⁶δ, ppm) 8.15 (d, 1H), 7.81-7.77 (m, 1H), 7.63-7.59 (m, 1H), 7.54-7.48 (m, 3H), 7.43-7.30 (m, 5H), 7.22-7.19 (m, 1H), 6.44 (s, 1H), 5.24 (s, 2H).

Synthesis stage 7:5-[3-(Benzyloxy)-4-bromo-1-(2-fluorophenyl)-1H-pyrazol-5-yl]-2-fluoropyridine

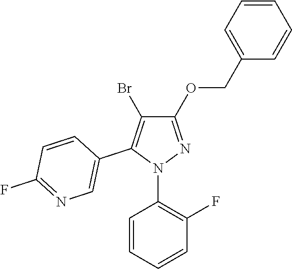 [SMILES: Fc1ccc(-c2c(Br)c(OCc3ccccc3)nn2-c2ccccc2F)cn1]

5-[3-(Benzyloxy)-1-(2-fluorophenyl)-1H-pyrazol-5-yl]-2-fluoropyridine (870 mg, 1.92 mmol, 1.0 equiv, 80% pure) was dissolved in DMF (10 ml), and 1-bromopyrrolidine-2,5-dione (0.51 g, 2.87 mmol, 1.5 equiv) was added at room temperature. The resulting reaction mixture was stirred at room temperature overnight and then admixed with ice-water (50 ml), which resulted in a precipitate. The precipitate was dissolved in dichloromethane (100 ml) and extracted with saturated sodium thiosulfate solution. The organic phase was dried over magnesium sulfate and the solvent was removed under reduced pressure. By final purification of the resulting crude product by column chromatography (ethyl acetate/heptane gradient), 5-[3-(benzyloxy)-4-bromo-1-(2-fluorophenyl)-1H-pyrazol-5-yl]-2-fluoropyridine was isolated in the form of a white solid (847 mg, 99% of theory). ¹H-NMR (400 MHz, DMSO-d⁶δ, ppm) 8.20 (d, 1H), 7.96-7.91 (m, 1H), 7.61 (dt, 1H), 7.52-7.27 (m, 9H), 5.33 (s, 2H).

Synthesis stage 8:4-Bromo-1-(2-fluorophenyl)-5-(6-fluoropyridin-3-yl)-1H-pyrazol-3-ol

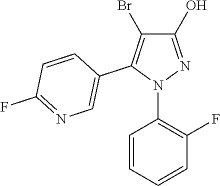 [SMILES: Oc1nn(-c2ccccc2F)c(-c2ccc(F)nc2)c1Br]

5-[3-(Benzyloxy)-4-bromo-1-(2-fluorophenyl)-1H-pyrazol-5-yl]-2-fluoropyridine (811 mg, 1.83 mmol, 1.0 equiv) was dissolved in dichloromethane (25 ml), and trifluoromethanesulfonic acid (0.33 ml, 3.67 mmol, 2.0 equiv) was added at room temperature. The resulting reaction mixture was stirred at room temperature for 2 h and then extracted with a saturated sodium hydrogencarbonate solution (20 ml). The aqueous phase was extracted twice with dichloromethane (25 ml). The combined organic phase was dried over magnesium sulfate, and the solvent was removed under reduced pressure. The crude product was used in the next synthesis stage without further purification. 4-Bromo-1-(2-fluorophenyl)-5-(6-fluoropyridin-3-yl)-1H-pyrazol-3-ol was isolated in the form of a white solid (700 mg, 92% of theory, 90% pure). ¹H-NMR (400 MHz, CDCl₃δ, ppm) 9.51 (bs, 1H), 8.12 (d, 1H), 7.76 (dt, 1H), 7.48-7.43 (m, 1H), 7.41-7.38 (m, 1H), 7.27-7.24 (m, 1H), 7.08-7.06 (m, 1H), 6.96-6.94 (m, 1H).

Synthesis stage 8:4-Bromo-1-(2-fluorophenyl)-5-(6-fluoropyridin-3-yl)-1H-pyrazole-3-thiol

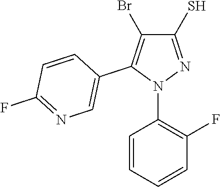 [SMILES: Fc1ccc(-c2c(Br)c(S)nn2-c2ccccc2F)cn1]

4-Bromo-1-(2-fluorophenyl)-5-(6-fluoropyridin-3-yl)-1H-pyrazol-3-ol (930 mg, 2.64 mmol, 1.0 equiv) was suspended in toluene (20 ml), and phosphorus pentasulfide (2.35 g, 5.28 mmol, 2.0 equiv) was added at room temperature. The resulting reaction mixture was heated to boiling for 6 h (evolution of gas). After cooling to room temperature, the precipitate was filtered off, and this was extracted by stirring with toluene (50 ml). The toluene phases were combined, and the solvent was removed under reduced pressure. The crude product was used in the next synthesis stage without further purification. 4-Bromo-1-(2-fluorophenyl)-5-(6-fluoropyridin-3-yl)-1H-pyrazole-3-thiol was isolated in the form of a yellow solid (515 mg, 52% of theory). ¹H-NMR (400 MHz, CDCl₃δ, ppm)

Synthesis stage 9: Ethyl {[4-bromo-1-(2-fluorophenyl)-5-(6-fluoropyridin-3-yl)-1H-pyrazol-3-yl]sulfanyl}acetate (Example No. II-001)

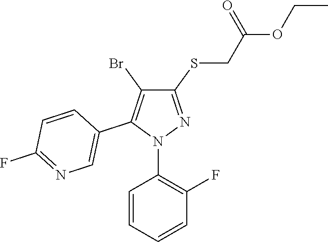 [SMILES: CCOC(=O)CSc1nn(-c2ccccc2F)c(-c2ccc(F)nc2)c1Br]

4-Bromo-1-(2-fluorophenyl)-5-(6-fluoropyridin-3-yl)-1H-pyrazole-3-thiol (515 mg, 1.40 mmol, 1.0 equiv) and sodium acetate (172 mg, 2.10 mmol, 1.5 equiv) was suspended in ethanol (10 ml), and then ethyl bromoacetate (0.17 ml, 1.54 mmol, 1.1 equiv) was added. The resulting reaction mixture was heated to boiling for 3 h. After cooling to room temperature, the solvent was removed under reduced pressure, and the residue was extracted with dichloromethane (50 ml) and water (10 ml), then stirred with toluene (50 ml). The organic phase was dried over magnesium sulfate and the solvent was removed under reduced pressure. By final purification of the resulting crude product by column chromatography (ethyl acetate/heptane gradient), ethyl {[4-bromo-1-(2-fluorophenyl)-5-(6-fluoropyridin-3-yl)-1H-pyrazol yl]sulfanyl}acetate was isolated in the form of a white solid (21 mg, 3% of theory). ¹H-NMR (400 MHz, CDCl₃δ, ppm) 8.10 (d, 1H), 7.72 (dt, 1H), 7.48-7.42 (m, 1H), 7.41-7.37 (m, 1H), 7.27-7.23 (m, 1H), 7.07-7.03 (m, 1H), 6.97-6.92 (m, 1H), 4.22 (q, 2H), 3.90 (s, 2H), 1.25 (t, 3H).

In analogy to the preparation examples cited above and recited at the appropriate point, and taking account of the general details relating to the preparation of substituted pyrazoles, the compounds cited below are obtained:

TABLE I

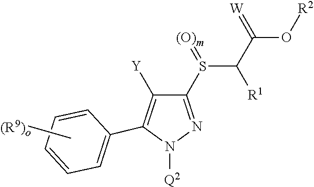

| Example | | | | | | |
|---|---|---|---|---|---|---|
| number | R¹ | R² | Y | Q² | m | (R⁹)ₒ |
| I-001 | H | Et | Cl | 2-F—Ph | 0 | 3,4-DiF |
| I-002 | H | Et | Br | 2-F—Ph | 0 | 3,4-DiF |
| I-003 | H | Et | Br | 2-F—Ph | 0 | 3-Cl-5-F |
| I-004 | H | Et | Br | 2,3-DiF—Ph | 0 | H |
| I-005 | H | H | Br | 2-F—Ph | 0 | 3,4-DiF |
| I-006 | H | H | Br | 2,3-DiF—Ph | 0 | H |
| I-007 | H | Et | Br | 2-F—Ph | 1 | 3,4-DiF |
| I-008 | H | Et | Br | 2,3-DiF—Ph | 2 | H |
| I-009 | H | H | Br | 2-F—Ph | 0 | 3-Cl-5-F |
| I-010 | H | Et | Br | 2,3-DiF—Ph | 1 | H |
| I-011 | H | Et | Br | 2-F—Ph | 0 | 4-I |
| I-012 | H | Et | CN | 2-F—Ph | 0 | 3,4-DiF |
| I-013 | H | Et | Br | 2,5-DiF | 0 | 3,4-DiF |
| I-014 | H | H | Cl | 2-F—Ph | 0 | 3,4-DiF |
| I-015 | Me | Et | Br | 2-F—Ph | 0 | 3,4-DiF |
| I-016 | H | Et | Br | 2,4-DiF—Ph | 0 | 4-F |
| I-017 | H | H | Br | 2-F—Ph | 0 | 4-I |
| I-018 | H | Et | CF₃ | 2-F—Ph | 0 | 3,4-DiF |
| I-019 | H | Et | Br | 4-F—Ph | 0 | 4-F |
| I-020 | H | Et | Br | 2,5-DiF—Ph | 0 | 4-F |
| I-021 | H | Et | Br | 2,3-DiCl—Ph | 0 | 4-F |
| I-022 | Me | H | Br | 2-F—Ph | 0 | 3,4-DiF |
| I-023 | H | Et | Br | 2-H₃CO—Ph | 0 | 4-F |
| I-024 | H | H | NO₂ | 2-F—Ph | 0 | 3,4-DiF |
| I-025 | H | Et | Br | 2,4-DiCl—Ph | 0 | 2,4-DiF |
| I-026 | H | Et | Br | 2,4-DiCl—Ph | 0 | 3,4-DiF |
| I-027 | H | Et | Br | 2,4-DiF—Ph | 0 | 3,4-DiF |

TABLE II

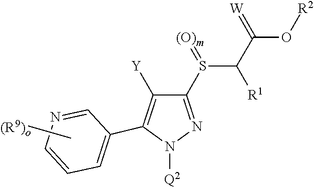

| Example | | | | | | |
|---|---|---|---|---|---|---|
| number | R¹ | R² | Y | Q² | m | (R⁹)ₒ |
| II-001 | H | Et | Br | 2-F—Ph | 0 | 6-F |
| II-002 | H | Et | Br | 2-F—Ph | 0 | 5-F |
| II-003 | H | Et | Br | 2-F—Ph | 2 | 5-F |
| II-004 | H | Et | Br | 2-F—Ph | 0 | 6-OH |
| II-005 | H | H | Br | 2-F—Ph | 0 | 5-F |
| II-006 | H | H | Br | 2-F—Ph | 0 | 6-OH |
| II-007 | H | Et | Br | 2-F—Ph | 0 | 5-Cl |
| II-008 | H | H | Br | 2-F—Ph | 0 | 5-Cl |
| II-009 | H | Et | Br | 2-F—Ph | 2 | 6-OH |
| II-010 | H | Et | Br | 2-F—Ph | 1 | 6-OH |
| II-011 | H | Et | Br | 2-F—Ph | 1 | 5-F |
| II-012 | H | Et | Br | 2-F—Ph | 0 | 6-Cl |
| II-013 | H | Et | Br | 2-F—Ph | 1 | 5-Cl |
| II-014 | H | Et | Br | 2-F—Ph | 0 | H |
| II-015 | H | Et | Br | 2-F—Ph | 0 | 6-Br |
| II-016 | H | H | Br | 2-F—Ph | 0 | 6-Cl |
| II-017 | H | H | Br | 2-F—Ph | 0 | H |

TABLE III

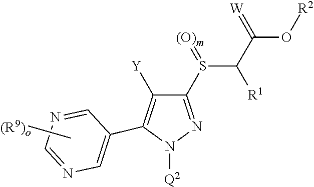

| Example | | | | | | |
|---|---|---|---|---|---|---|
| number | R¹ | R² | Y | Q² | m | (R⁹)ₒ |
| III-001 | H | Et | Br | 2-F—Ph | 0 | H |
| III-002 | H | H | Br | Ph | 0 | H |
| III-003 | H | Et | Br | Ph | 0 | H |
| III-004 | H | Et | Br | 2,3-DiF—Ph | 0 | H |
| III-005 | H | H | Br | 2,3-DiF—Ph | 0 | H |

TABLE IV

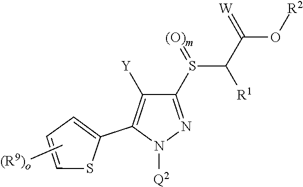

| Example | | | | | | |
|---|---|---|---|---|---|---|
| number | R¹ | R² | Y | Q² | m | (R⁹)ₒ |
| IV-001 | H | Et | Br | 2-F—Ph | 0 | 4-Cl |

TABLE V

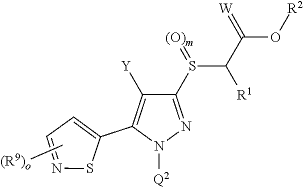

| Example | | | | | | |
|---|---|---|---|---|---|---|
| number | R¹ | R² | Y | Q² | m | (R⁹)ₒ |
| V-001 | H | Et | Br | 2-F—Ph | 0 | 4-Me |
| V-002 | H | H | Br | 2-F—Ph | 0 | 4-Me |

TABLE VI

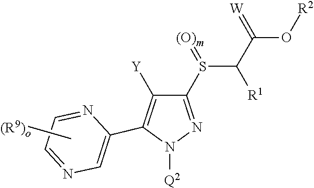

| Example | | | | | | |
|---|---|---|---|---|---|---|
| number | R¹ | R² | Y | Q² | m | (R⁹)ₒ |
| VI-001 | H | Et | Br | 2-F—Ph | 0 | H |
| VI-002 | H | H | Br | 2-F—Ph | 0 | H |

TABLE VII

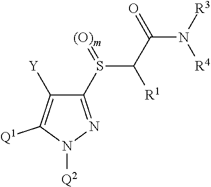

| Example | | | | | | | |
|---|---|---|---|---|---|---|---|
| number | R¹ | Y | Q¹ | Q² | m | R³ | R⁴ |
| VII-001 | H | Cl | 3,4-DiF—Ph | 2-F—Ph | 0 | H | CH₂CO₂CH₃ |
| VII-002 | H | Cl | 3,4-DiF—Ph | 2-F—Ph | 0 | Me | CH₂CO₂CH₃ |
| VII-003 | H | Br | 3,4-DiF—Ph | 2-F—Ph | 0 | H | CH₂CO₂CH₂CH₃ |
| VII-004 | H | NO₂ | 3,4-DiF—Ph | 2-F—Ph | 0 | H | CH₂CO₂CH₃ |
| VII-005 | H | Br | Ph | 2-F—Ph | 0 | H | CH₂CO₂CH₃ |
| VII-006 | H | Br | 3-pyridyl | 2-F—Ph | 0 | H | CH₂CH₂CO₂CH₃ |
| VII-007 | H | Br | 3-pyridyl | 2-F—Ph | 0 | H | CH₂CO₂CH₃ |
| VII-008 | H | Br | 3-pyridyl | 2-F—Ph | 0 | Me | CH₂CO₂CH₃ |
| VII-009 | H | Br | 5-F-3-Pyridyl | 2-F—Ph | 0 | H | CH₂CO₂CH₃ |
| VII-010 | H | Br | 5-F-3-Pyridyl | 2-F—Ph | 0 | H | CH₂CH₂CO₂CH₃ |
| VII-011 | H | Br | 3,4-DiF—Ph | 2-F—Ph | 0 | Me | CH₂CO₂CH₃ |

TABLE VIII

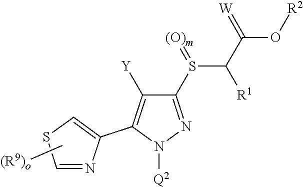

| Example | | | | | | |
|---|---|---|---|---|---|---|
| number | R¹ | R² | Y | Q² | m | (R⁹)ₒ |
| VIII-001 | H | Et | Br | 2-F—Ph | 0 | 2-Cl |

Spectroscopic data of selected table examples:

Selected detailed synthesis examples for the inventive compounds of the general formula (I) are adduced below. The ¹H NMR, ¹³C-NMR and ¹⁹F-NMR spectroscopy data reported for the chemical examples described in the sections which follow (400 MHz for ¹H NMR and 150 MHz for ¹³C-NMR and 375 MHz for ¹⁹F-NMR, solvent CDCl₃, CD₃OD or d₆-DMSO, internal standard: tetramethylsilane δ=0.00 ppm) were obtained on a Bruker instrument, and the signals listed have the meanings given below: br=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets. In the case of diastereomer mixtures, either the significant signals for each of the two diastereomers are reported or the characteristic signal of the main diastereomer is reported. The abbreviations used for chemical groups have, for example, the following meanings: Me=CH₃, Et=CH₂CH₃, t-Hex=C(CH₃)₂CH(CH₃)₂, t-Bu=C(CH₃)₃, n-Bu=unbranched butyl, n-Pr=unbranched propyl, i-Pr=branched propyl, c-Pr=cyclopropyl, c-Hex=cyclohexyl.

The spectroscopic data listed hereinafter for selected table examples were evaluated via conventional ¹H NMR interpretation or via NMR peak list methods.

Conventional ¹H NMR Interpretation

### Synthesis Example No. I-001

¹H-NMR (400 MHz, CDCl₃, δ, ppm) 7.42 (m, 2H), 7.25 (m, 1H), 7.12 (m, 2H), 7.07 (m, 1H), 4.20 (q, 2H), 3.87 (s, 2H), 1.24 (t, 3H).

### Synthesis Example No. 1-003

¹H-NMR (400 MHz, CDCl₃, δ, ppm) 8.46 (d, 1H), 8.27 (d, 1H), 7.47 (m, 1H), 7.39 (m, 2H), 7.26 (m, 1H), 7.05 (m, 1H), 4.22 (q, 2H), 3.90 (s, 2H), 1.26 (t, 3H).

### Synthesis Example No. 1-004

¹H-NMR (400 MHz, CDCl₃, δ, ppm) 7.35 (m, 3H), 7.26 (m, 2H), 7.12 (m, 3H), 4.22 (q, 2H), 3.90 (s, 2H), 1.26 (t, 3H).

### Synthesis Example No. 1-006

¹H-NMR (400 MHz, DMSO-d⁶, 6, ppm) 7.57 (m, 1H), 7.45-7.28 (m, 7H), 3.96 (s, 2H).

### Synthesis Example No. 1-007

¹H-NMR (400 MHz, CDCl₃, δ, ppm) 7.44 (m, 2H), 7.26 (m, 1H), 7.12 (m, 3H), 6.99 (m, 1H), 4.40 (s, 2H), 4.26 (q, 2H), 1.27 (t, 3H).

### Synthesis Example No. 1-009

¹H-NMR (400 MHz, CDCl₃, δ, ppm) 10.80 (br s, 1H), 7.42 (m, 2H), 7.25 (m, 1H), 7.11 (m, 3H), 3.87 (s, 2H).

### Synthesis Example No. I-010

¹H-NMR (400 MHz, CDCl₃, δ, ppm) 7.64 (m, 1H), 7.47 (m, 4H), 7.33 (m, 3H), 4.55 (q, 2H), 4.15 (m, 2H), 1.16 (t, 3H).

### Synthesis Example No. 11-002

¹H-NMR (400 MHz, CDCl₃, δ, ppm) 8.45 (d, 1H), 8.27 (d, 1H), 7.46 (m, 1H), 7.42-7.36 (m, 2H), 7.27 (m, 1H), 7.05 (m, 1H), 4.22 (q, 2H), 3.90 (s, 2H), 1.25 (t, 3H).

### Synthesis Example No. 111-002

¹H-NMR (400 MHz, CDCl₃, δ, ppm) 9.23 (s, 1H), 8.67 (s, 2H), 7.40 (m, 3H), 7.18 (m, 2H), 3.90 (s, 2H).

### Synthesis Example No. 111-003

¹H-NMR (400 MHz, DMSO-d⁶, 6, ppm) 9.23 (s, 1H), 8.77 (s, 2H), 7.43 (m, 3H), 7.29 (m, 2H), 4.13 (q, 2H), 4.09 (s, 2H), 1.16 (t, 3H).

### Synthesis Example No. IV-001

¹H-NMR (400 MHz, CDCl₃, δ, ppm) 7.44 (m, 2H), 7.26 (m, 1H), 7.16 (m, 1H), 6.86 (d, 1H), 6.81 (d, 1H), 4.19 (q, 2H), 3.86 (s, 2H), 1.23 (t, 3H).

### Synthesis Example No. V-001

¹H-NMR (400 MHz, CDCl₃, δ, ppm) 7.43 (m, 1H), 7.31 (m, 1H), 7.29 (m, 1H), 7.18 (m, 1H), 7.10 (s, 1H), 4.20 (q, 2H), 3.87 (s, 2H), 2.25 (s, 3H), 1.24 (t, 3H).

### Synthesis Example No. V-002

¹H-NMR (400 MHz, CDCl₃, δ, ppm) 7.53 (m, 1H), 7.43 (m, 1H), 7.32 (m, 1H), 7.21 (m, 1H), 7.13 (s, 1H), 3.86 (s, 2H), 2.46 (s, 3H).

### Synthesis Example No. 11-003

¹H-NMR (400 MHz, CDCl₃, δ, ppm) 8.50 (d, 1H), 8.28 (s, 1H), 7.53-7.39 (m, 3H), 7.28 (m, 1H), 7.09 (m, 1H), 4.41 (s, 2H), 4.24 (m, 2H), 1.27 (t, 3H).

### Synthesis Example No. 11-004

¹H-NMR (400 MHz, CDCl₃, δ, ppm) 11.09 (br s, 1H), 8.25 (s, 1H), 7.57-7.51 (m, 2H), 7.46-7.42 (m, 1H), 7.38 (dd, 1H), 7.31-7.25 (m, 2H), 4.07 (q, 2H), 4.01 (s, 2H), 1.10 (t, 3H).

### Synthesis Example No. I-011

¹H-NMR (400 MHz, CDCl₃, δ, ppm) 7.80 (d, 2H), 7.57-7.48 (m, 2H), 7.34-7.28 (m, 2H), 7.05 (d, 2H), 4.09 (q, 2H), 3.97 (s, 2H), 1.11 (t, 3H).

### Synthesis Example No. 11-005

¹H-NMR (400 MHz, CDCl₃, δ, ppm) 8.50 (d, 1H), 8.29 (d, 1H), 7.47-7.39 (m, 3H), 7.27 (m, 1H), 7.10-7.05 (m, 1H), 3.91 (s, 2H).

### Synthesis Example No. 11-006

¹H-NMR (400 MHz, CDCl₃, δ, ppm) 11.09 (br s, 1H), 8.29 (d, 1H), 7.56-7.53 (m, 2H), 7.50-7.44 (m, 2H), 7.36-7.27 (m, 2H), 3.96 (s, 2H).

### Synthesis Example No. 11-007

¹H-NMR (400 MHz, CDCl₃, δ, ppm) 8.54 (s, 1H), 8.31 (s, 1H), 7.66 (s, 1H), 711H)111117.49-7.36 (m, 2H), 7.27-7.23 (m, 1H), 7.08-7.03 (m, 1H), 4.21 (q, 2H), 3.90 (s, 2H), 1.26 (t, 3H).

### Synthesis Example No. 1-012

¹H-NMR (400 MHz, CDCl₃, δ, ppm) 711H)111117.48-7.43 (m, 2H), 7.27-7.26 (m, 1H), 7.20-7.07 (m, 4H), 4.21 (q, 2H), 3.93 (s, 2H), 1.26 (t, 3H).

### Synthesis Example No. 11-008

¹H-NMR (400 MHz, CDCl₃, δ, ppm) 8.54 (s, 1H), 8.31 (s, 1H), 7.66 (s, 1H), 711H)111117.49-7.36 (m, 2H), 7.27-7.23 (m, 1H), 7.08-7.03 (m, 1H), 3.92 (s, 2H).

### Synthesis Example No. 11-009

¹H-NMR (400 MHz, CDCl₃, δ, ppm) 11.26 (s, 1H), 8.71 (m, 2H), 8.09 (s, 1H), 7.66-7.59 (m, 1H), 711H)111117.50-7.43 (m, 1H), 7.37-7.24 (m, 2H), 7.08-7.03 (m, 1H), 4.77 (s, 2H), 3.95 (q, 2H), 0.95 (t, 3H).

### Synthesis Example No. II-010

¹H-NMR (400 MHz, CDCl₃, δ, ppm) 11.26 (s, 1H), 8.59 (d, 1H), 8.01 (d, 1H), 7.91 (d, 1H), 7.61-7.56 (m, 1H), 711H)111117.48-7.43 (m, 1H), 7.32-7.24 (m, 2H), 4.25-4.22 (d, 1H), 4.03-3.96 (m, 3H), 1.04 (t, 3H).

### Synthesis Example No. 1-013

¹H-NMR (400 MHz, CDCl₃, δ, ppm) 7.46-7.35 (m, 2H), 711H)111117.27-7.18 (m, 1H), 7.17-7.02 (m, 2H), 6.97-6.92 (m, 1H), 4.22 (q, 2H), 3.89 (s, 2H), 1.24 (t, 3H).

### Synthesis Example No. II-011

¹H-NMR (400 MHz, CDCl₃, δ, ppm) 8.51 (s, 1H), 8.28 (s, 1H), 711H)111117.53-7.38 (m, 3H), 7.32-7.26 (m, 1H), 7.12-7.07 (m, 1H), 4.40 (d, 2H), 4.25 (q, 2H), 1.27 (t, 3H).

### Synthesis Example No. 11-014

¹H-NMR (400 MHz, CDCl₃, δ, ppm) 7.80 (d, 2H), 7.57-7.48 (m, 2H), 7.34-7.28 (m, 2H), 7.05 (d, 2H), 3.97 (s, 2H).

### Synthesis Example No. 111-004

¹H-NMR (400 MHz, CDCl₃, δ, ppm) 9.22 (s, 1H), 8.67 (s, 2H), 7.25-7.22 (m, 3H), 4.22 (q, 2H), 3.91 (s, 2H), 1.26 (t, 3H).

### Synthesis Example No. 1-015

¹H-NMR (400 MHz, CDCl₃, δ, ppm) 7.42-7.36 (m, 2H), 7.22 (m, 1H), 7.16-6.95 (m, 4H), 4.21-4.13 (m, 3H), 1.62 (d, 3H), 1.22 (t, 3H).

### Synthesis Example No. VI-001

¹H-NMR (400 MHz, CDCl₃, δ, ppm) 8.94 (s, 1H), 8.68 (m, 1H), 8.59 (m, 1H), 7.55-7.46 (m, 2H), 7.34-7.27 (m, 2H), 4.10 (q, 2H), 4.01 (s, 2H), 1.12 (t, 3H).

### Synthesis Example No. 11-012

¹H-NMR (400 MHz, CDCl₃, δ, ppm) 8.24 (m, 1H), 7.60 (dd, 1H), 7.46 (dt, 1H), 7.37 (m, 1H), 7.33 (m, 1H), 7.23 (m, 1H), 7.05 (dt, 1H), 4.21 (q, 2H), 3.89 (s, 2H), 1.25 (t, 3H).

### Synthesis Example No. 1-016

¹H-NMR (400 MHz, CDCl₃, δ, ppm) 7.43 (m, 1H), 7.25-6.95 (m, 3H), 6.84 (m, 1H), 4.38 (s, 2H), 4.25 (q, 2H), 1.27 (t, 3H).

### Synthesis Example No. 11-013

¹H-NMR (400 MHz, CDCl₃, δ, ppm) 8.59 (m, 1H), 8.32 (m, 1H), 7.66 (m, 1H), 7.53-7.44 (m, 2H), 7.30 (m, 1H), 7.10 (m, 1H), 4.40 (s, 2H), 4.25 (q, 2H), 1.27 (t, 3H).

### Synthesis Example No. 1-019

¹H-NMR (400 MHz, CDCl₃, δ, ppm) 7.25-7.22 (m, 2H), 7.17-7.13 (m, 2H), 7.10-7.05 (m, 2H), 7.01-6.97 (m, 2H), 4.22 (q, 2H), 3.89 (s, 2H), 1.26 (t, 3H).

### Synthesis Example No. 1-017

¹H-NMR (400 MHz, CDCl₃, δ, ppm) 12.81 (bs, 1H), 7.78 (d, 2H), 7.59-7.48 (m, 2H), 7.33-7.29 (m, 2H), 7.07 (d, 2H), 3.92 (s, 2H).

### Synthesis Example No. 1-020

¹H-NMR (400 MHz, CDCl₃, δ, ppm) 7.25-7.23 (m, 2H), 7.20-7.16 (m, 1H), 7.07-6.95 (m, 4H), 4.22 (q, 2H), 3.89 (s, 2H), 1.26 (t, 3H).

### Synthesis Example No. I-021

¹H-NMR (400 MHz, CDCl₃, δ, ppm) 7.41 (m, 1H), 7.27 (m, 1H), 7.24-7.20 (m, 2H), 7.04-7.00 (m, 2H), 4.20 (q, 2H), 3.88 (s, 2H), 1.25 (t, 3H).

### Synthesis Example No. 1-022

¹H-NMR (400 MHz, d⁶-DMSO, δ, ppm) 7.71-7.12 (m, 7H), 4.15 (m, 1H), 1.52 (d, 3H).

### Synthesis Example No. 1-023

¹H-NMR (400 MHz, CDCl₃, δ, ppm) 7.39-7.30 (m, 2H), 7.24-7.20 (m, 2H), 7.02-6.95 (m, 3H), 6.79 (d, 1H), 4.20 (q, 2H), 3.87 (s, 2H), 3.43 (s, 3H), 1.25 (t, 3H).

### Synthesis Example No. 1-024

¹H-NMR (400 MHz, d⁶-DMSO, δ, ppm) 12.85 (bs, 1H), 7.75 (m, 1H), 7.64 (m, 1H), 7.58-7.45 (m, 2H), 7.37-7.28 (m, 3H), 4.02 (s, 2H).

### Synthesis Example No. 11-015

¹H-NMR (400 MHz, CDCl₃, δ, ppm) 8.22 (m, 1H), 7.49-7.38 (m, 4H), 7.26 (m, 1H, 7.05 (dt, 1H), 4.20 (q, 2H), 3.89 (s, 2H), 1.24 (t, 3H).

### Synthesis Example No. 1-016

¹H-NMR (400 MHz, CDCl₃, δ, ppm) 8.26 (m, 1H), 7.60 (dd, 1H), 7.45-7.40 (m, 2H), 7.35 (d, 1H), 7.26 (m, 1H), 7.10 (m, 1H), 3.89 (s, 2H).

### Synthesis Example No. 1-025

¹H-NMR (400 MHz, CDCl₃, δ, ppm) 7.38 (m, 1H), 7.31-7.21 (m, 3H), 6.90 (m, 1H), 6.80 (m, 1H), 4.20 (q, 2H), 3.88 (s, 2H), 1.24 (t, 3H).

### Synthesis Example No. 1-026

¹H-NMR (400 MHz, CDCl₃, δ, ppm) 7.42 (m, 1H), 7.32-7.26 (m, 2H), 7.15-7.08 (m, 2H) 6.96-6.93 (m, 1H), 4.19 (q, 2H), 3.87 (s, 2H), 1.24 (t, 3H).

### Synthesis Example No. 1-027

¹H-NMR (400 MHz, CDCl₃, δ, ppm) 7.39 (m, 1H), 7.17-7.08 (m, 2H), 6.98-6.93 (m, 2H), 6.81 (m, 1H), 4.20 (q, 2H), 3.88 (s, 2H), 1.25 (t, 3H).

### Synthesis Example No. VII-001

¹H-NMR (400 MHz, CDCl₃, δ, ppm) 7.55 (bs, 1H), 7.47-7.39 (m, 2H), 7.25 (m, 1H), 7.17-7.06 (m, 3H), 6.99 (m, 1H), 4.05 (d, 2H), 3.81 (s, 2H), 3.71 (s, 3H).

### Synthesis Example No. 1-014

¹H-NMR (400 MHz, CDCl₃, δ, ppm) 7.46-7.39 (m, 2H), 7.25 (m, 1H), 7.14-7.10 (m, 3H), 6.97 (m, 1H), 3.86 (s, 2H).

### Synthesis Example No. VII-002

¹H-NMR (400 MHz, CDCl₃, δ, ppm) 7.47-7.36 (m, 2H), 7.25 (m, 1H), 7.15-7.04 (m, 3H), 6.97 (m, 1H), 4.16 (s, 2H), 4,10 (s, 2H), 3.73 (s, 3H), 3.18 (s, 3H).

### Synthesis Example No. VIII-001

¹H-NMR (400 MHz, CDCl₃, δ, ppm) 7.53-7.42 (m, 2H), 7.28 (m, 1H), 6.85 (d, 1H), 4.20 (q, 2H), 3.80 (s, 2H), 1.25 (t, 3H).

### Synthesis Example No. VII-003

¹H-NMR (400 MHz, CDCl₃, δ, ppm) 7.52 (bt, 1H), 7.47-7.36 (m, 2H), 7.23 (m, 1H), 7.18-7.04 (m, 3H), 7.00 (m, 1H), 4.17 (q, 2H), 4.03 (d, 2H), 3.82 (s, 2H), 1.25 (t, 3H).

### Synthesis Example No. VII-004

¹H-NMR (400 MHz, CDCl₃, δ, ppm) 7.47-7.41 (m, 2H), 7.25-7.02 (m, 6H), 4.05 (d, 2H), 3.89 (s, 2H), 3.72 (s, 3H).

NMR Peak List Method

The 1H NMR data of selected examples are noted in the form of 1H NMR peak lists. For each signal peak, first the 6 value in ppm and then the signal intensity in round brackets are listed. The 6 value/signal intensity number pairs for different signal peaks are listed with separation from one another by semicolons.

The peak list for one example therefore takes the form of:

δ₁(intensity₁);δ₂(intensity₂); . . . ;δᵢ;(intensityᵢ); . . . ;δₙ(intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

For calibration of the chemical shift of 1H NMR spectra we use tetramethylsilane and/or the chemical shift of the solvent, particularly in the case of spectra measured in DMSO. Therefore, the tetramethylsilane peak may but need not occur in NMR peak lists.

The lists of the 1H NMR peaks are similar to the conventional 1H NMR printouts and thus usually contain all peaks listed in a conventional NMR interpretation.

In addition, like conventional 1H NMR printouts, they may show solvent signals, signals of stereoisomers of the target compounds, which likewise form part of the subject matter of the invention, and/or peaks of impurities.

In the reporting of compound signals in the delta range of solvents and/or water, our lists of 1H NMR peaks show the usual solvent peaks, for example peaks of DMSO in DMSO-D₆ and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the relevant peak picking in conventional 1H NMR interpretation.

Further details of 1H NMR peak lists can be found in the Research Disclosure Database Number 564025.

VI-002: ¹H-NMR (400.6 MHz, d₆-DMSO):
δ = 12.8357 (0.6); 8.9379 (3.3); 8.9343 (3.4); 8.6831 (3.2); 8.6772 (3.8); 8.6002 (2.7); 8.5963 (2.8); 8.5939 (2.5); 8.5901 (2.4);
7.5822 (0.6); 7.5779 (0.7); 7.5628 (1.3); 7.5584 (1.5); 7.5434 (0.8); 7.5390 (0.8); 7.5006 (0.6); 7.4958 (0.6); 7.4879 (0.6);
7.4835 (0.6); 7.4756 (0.5); 7.4673 (0.5); 7.3431 (0.8); 7.3406 (1.0); 7.3242 (1.2); 7.3210 (1.6); 7.3107 (1.0); 7.3078 (0.8);
7.3018 (0.8); 7.2900 (0.8); 7.2842 (1.0); 7.2811 (0.8); 7.2633 (0.7); 7.2602 (0.6); 5.7568 (16.0); 3.9748 (10.0); 3.3213 (18.7);
2.6744 (0.8); 2.6698 (1.1); 2.6652 (0.8); 2.5404 (1.1); 2.5236 (3.3); 2.5189 (4.3); 2.5101 (60.8); 2.5056 (133.6); 2.5010
(187.3); 2.4964 (129.6); 2.4918 (58.7); 2.4642 (0.8); 2.3327 (0.8); 2.3280 (1.1); 2.3235 (0.8); 1.9885 (0.7); 1.1689 (0.5);
0.0103 (0.6); 0.0080 (4.4); 0.0064 (1.2); 0.0056 (1.3); 0.0047 (1.7); 0.0039 (2.4); −0.0002 (151.4); −0.0050 (2.1); −0.0058 (1.8);
−0.0067 (1.6); −0.0085 (4.4); −0.0106 (0.8); −0.0114 (0.7); −0.0122 (0.6)
VII-005: ¹H-NMR (400.6 MHz, CDCl3):
δ = 7.3896 (0.6); 7.3866 (0.7); 7.3797 (0.6); 7.3768 (1.0); 7.3724 (3.8); 7.3699 (1.4); 7.3674 (1.8); 7.3623 (0.5); 7.3578 (0.9);
7.3539 (2.2); 7.3391 (0.8); 7.3315 (0.6); 7.2773 (2.3); 7.2735 (2.3); 7.2604 (19.9); 7.2533 (1.5); 7.2015 (0.7); 7.1830 (0.6);
7.1592 (0.5); 7.1453 (0.7); 7.1416 (0.8); 7.1306 (0.7); 7.1287 (0.8); 7.1223 (1.1); 7.1115 (0.6); 7.1079 (0.6); 7.0999 (0.6);
7.0874 (0.6); 5.2998 (6.8); 4.0637 (3.9); 4.0505 (3.9); 3.8259 (8.2); 3.7125 (16.0); 2.0453 (0.6); 1.5502 (4.2); 1.2595 (0.5);
0.0079 (0.8); −0.0002 (29.7); −0.0085 (0.9)
II-017: ¹H-NMR (400.6 MHz, d₆-DMSO):
δ = 12.8229 (0.9); 8.5977 (3.0); 8.5936 (3.3); 8.5856 (3.3); 8.5815 (3.2); 8.4763 (3.0); 8.4742 (3.3); 8.4706 (3.4); 8.4685 (3.2);
7.7351 (1.6); 7.7309 (1.9); 7.7296 (1.8); 7.7252 (1.6); 7.7153 (1.9); 7.7111 (2.1); 7.7097 (2.3); 7.7054 (1.8); 7.6479 (1.0);
7.6437 (1.2); 7.6285 (2.0); 7.6242 (2.2); 7.6092 (1.2); 7.6048 (1.3); 7.5378 (0.6); 7.5334 (0.6); 7.5251 (0.7); 7.5189 (1.0);
7.5145 (0.9); 7.5061 (1.0); 7.5018 (1.0); 7.4983 (1.0); 7.4938 (0.8); 7.4855 (0.8); 7.4810 (0.8); 7.4662 (2.1); 7.4640 (2.1);
7.4541 (2.0); 7.4519 (2.0); 7.4464 (1.8); 7.4442 (1.9); 7.4343 (1.9); 7.4320 (1.9); 7.3629 (1.2); 7.3602 (1.5); 7.3440 (2.0);
7.3409 (2.4); 7.3209 (2.2); 7.3175 (1.3); 7.2997 (1.2); 7.2946 (1.6); 7.2913 (1.3); 7.2736 (1.1); 7.2705 (1.0); 4.0377 (1.0);
4.0199 (1.0); 3.9568 (16.0); 3.3233 (77.7); 2.6745 (1.0); 2.6699 (1.4); 2.6653 (1.0); 2.5405 (5.3); 2.5237 (4.0); 2.5190 (5.7);
2.5103 (72.3); 2.5057 (159.3); 2.5011 (224.2); 2.4965 (157.2); 2.4919 (71.2); 2.4698 (0.7); 2.3328 (0.9); 2.3282 (1.3); 2.3236
(0.9); 1.9885 (4.8); 1.9083 (1.1); 1.6520 (1.7); 1.3550 (3.2); 1.2353 (0.6); 1.1921 (1.5); 1.1744 (3.0); 1.1566 (1.4); 0.1458
(0.5); 0.0103 (0.6); 0.0080 (5.0); 0.0064 (1.2); 0.0056 (1.2); 0.0048 (1.5); 0.0039 (2.0); 0.0023 (6.3); −0.0002 (174.8); −0.0050
(3.1); −0.0058 (2.6); −0.0067 (2.1); −0.0085 (5.3); −0.0106 (1.1); −0.0114 (1.0); −0.0122 (0.9); −0.0138 (0.5); −0.0146 (0.5);
−0.1493 (0.5)
III-005: ¹H-NMR (400.6 MHz, d₆-DMSO):
δ = 12.8568 (0.7); 9.2554 (7.2); 8.8381 (16.0); 7.6143 (0.7); 7.5956 (0.6); 7.4214 (0.7); 7.4175 (1.0); 7.4023 (0.8); 7.3846 (0.5);
7.3806 (0.6); 7.3719 (0.5); 7.3643 (0.6); 7.3599 (0.7); 7.3511 (0.6); 7.3467 (0.6); 6.8702 (0.6); 5.7567 (1.4); 3.9818 (9.2);
3.3199 (73.7); 2.6790 (0.6); 2.6745 (1.3); 2.6698 (1.8); 2.6651 (1.3); 2.6605 (0.6); 2.5404 (6.8); 2.5326 (1.4); 2.5280 (1.6);
2.5236 (5.4); 2.5189 (6.6); 2.5101 (98.2); 2.5056 (220.8); 2.5010 (313.5); 2.4964 (221.6); 2.4918 (101.7); 2.4713 (0.6); 2.4663
(0.7); 2.4614 (0.8); 2.4567 (0.8); 2.3373 (0.6); 2.3326 (1.4); 2.3280 (1.9); 2.3234 (1.4); 2.3188 (0.7); 2.1826 (1.0); 1.9885
(1.5); 1.9081 (0.8); 1.3549 (8.7); 1.2352 (1.9); 1.1744 (1.0); 0.1457 (0.7); 0.0317 (0.5); 0.0080 (6.1); 0.0063 (1.2); 0.0055
(1.3); 0.0046 (1.6); −0.0002 (226.6); −0.0051 (4.1); −0.0059 (3.4); −0.0068 (3.0); −0.0085 (7.2); −0.0139 (0.8); −0.0179 (0.5);
−0.1494 (0.7)
VII-006: ¹H-NMR (400.6 MHz, CDCl3):
δ = 8.6037 (1.0); 8.5996 (1.0); 8.5916 (1.0); 8.5875 (1.0); 8.5004 (1.3); 8.4964 (1.3); 7.6568 (0.7); 7.6523 (0.9); 7.6469 (0.7);
7.6370 (0.8); 7.6324 (1.0); 7.6271 (0.7); 7.4838 (0.5); 7.4795 (0.6); 7.4645 (1.1); 7.4604 (1.2); 7.4457 (0.9); 7.4414 (0.9);
7.3832 (0.5); 7.3208 (0.8); 7.3189 (0.8); 7.3086 (0.8); 7.3067 (0.8); 7.3010 (0.7); 7.2991 (0.7); 7.2888 (0.7); 7.2868 (0.7);
7.2613 (18.0); 7.2550 (0.7); 7.2518 (0.7); 7.2333 (1.0); 7.0887 (0.6); 7.0855 (0.6); 7.0678 (0.6); 7.0641 (1.0); 7.0604 (0.7);
7.0428 (0.6); 7.0396 (0.6); 5.3002 (6.6); 4.1309 (0.7); 4.1131 (0.7); 3.7656 (8.2); 3.5734 (16.0); 3.5581 (0.9); 3.5427 (2.4);
3.5273 (2.5); 3.5117 (1.0); 2.5239 (1.8); 2.5085 (3.1); 2.4931 (1.7); 2.0454 (3.1); 1.5705 (1.0); 1.2774 (0.9); 1.2596 (1.8);
1.2417 (0.8); 0.0080 (0.7); −0.0002 (27.4); −0.0085 (0.8)
VII-007: ¹H-NMR (400.6 MHz, CDCl3):
δ = 8.6051 (0.9); 8.6010 (1.0); 8.5929 (1.0); 8.5888 (1.0); 8.4964 (1.2); 8.4926 (1.2); 7.6601 (0.6); 7.6556 (0.8); 7.6503 (0.6);
7.6403 (0.7); 7.6350 (0.9); 7.6304 (0.7); 7.4842 (0.5); 7.4799 (0.6); 7.4649 (0.9); 7.4607 (1.0); 7.4461 (0.6); 7.4418 (0.7);
7.3235 (0.7); 7.3214 (0.7); 7.3113 (0.7); 7.3093 (0.7); 7.3037 (0.6); 7.3017 (0.6); 7.2916 (0.6); 7.2605 (49.9); 7.2372 (0.6);
7.2186 (1.0); 7.0829 (0.6); 7.0797 (0.6); 7.0621 (0.6); 7.0582 (1.0); 7.0544 (0.6); 7.0370 (0.6); 7.0337 (0.5); 5.3001 (4.9);
4.0637 (3.8); 4.0504 (3.8); 3.8357 (8.0); 3.7110 (16.0); 2.0454 (2.1); 1.5474 (2.9); 1.2775 (0.6); 1.2597 (1.2); 1.2418 (0.6);
0.0080 (2.3); −0.0002 (75.2); −0.0085 (2.1)
VII-008: ¹H-NMR (400.6 MHz, CDCl3):
δ = 8.5875 (0.8); 8.5839 (0.8); 8.5754 (0.9); 8.5718 (0.8); 8.4805 (1.0); 8.4761 (1.3); 7.6535 (0.6); 7.6480 (0.9); 7.6436 (0.8);
7.6337 (0.7); 7.6282 (1.0); 7.6238 (0.8); 7.4739 (0.8); 7.4697 (0.8); 7.4553 (0.6); 7.4508 (0.6); 7.3633 (0.5); 7.3089 (0.8);
7.2968 (0.8); 7.2951 (0.8); 7.2892 (0.8); 7.2769 (0.7); 7.2753 (0.7); 7.2622 (12.6); 7.2449 (0.7); 7.2432 (0.8); 7.2237 (1.1);
7.0597 (0.6); 7.0566 (0.6); 7.0389 (0.6); 7.0352 (0.9); 7.0315 (0.6); 5.3002 (10.6); 4.2356 (1.6); 4.1673 (6.1); 4.1351 (8.0);
4.1309 (1.8); 4.1129 (1.2); 3.9867 (1.8); 3.7333 (16.0); 3.1909 (12.9); 3.0185 (2.9); 2.0451 (6.0); 1.2773 (1.6); 1.2595 (3.4);
1.2417 (1.6); −0.0002 (18.5); −0.0085 (0.6)
VII-009: ¹H-NMR (400.6 MHz, CDCl3):
δ = 8.4704 (2.1); 8.4635 (2.1); 8.2948 (1.1); 8.2910 (2.0); 8.2873 (1.1); 7.5201 (0.6); 7.5161 (0.6); 7.5010 (1.0); 7.4968 (1.2);
7.4822 (1.0); 7.4780 (1.0); 7.4207 (1.0); 7.4164 (1.2); 7.4139 (1.2); 7.4095 (0.9); 7.4058 (0.6); 7.3989 (1.1); 7.3943 (1.0);
7.3921 (1.0); 7.3875 (0.8); 7.3851 (0.5); 7.2690 (0.7); 7.2610 (26.4); 7.2494 (1.0); 7.1008 (0.7); 7.0976 (0.6); 7.0799 (0.6);
7.0761 (1.0); 7.0724 (0.7); 7.0548 (0.6); 7.0516 (0.6); 5.3002 (5.6); 4.0627 (3.9); 4.0495 (3.9); 3.8378 (8.0); 3.7142 (16.0);
2.0454 (1.2); 1.5671 (2.7); 1.2774 (0.6); 1.2597 (1.4); 0.8989 (0.6); 0.8820 (2.0); 0.8642 (0.8); 0.0080 (1.1); −0.0002 (39.8);
−0.0085 (1.1)
VII-010: ¹H-NMR (400.6 MHz, CDCl3):
δ = 8.4700 (1.9); 8.4631 (2.0); 8.2975 (1.0); 8.2937 (1.8); 8.2901 (1.0); 7.5105 (0.5); 7.4955 (0.8); 7.4913 (0.9); 7.4768 (0.6);
7.4724 (0.6); 7.4215 (1.2); 7.4170 (1.3); 7.4147 (1.1); 7.4103 (1.3); 7.4064 (0.7); 7.4045 (0.7); 7.4023 (0.8); 7.3995 (1.2);
7.3951 (1.2); 7.3926 (1.1); 7.3899 (0.8); 7.3882 (1.0); 7.3857 (0.6); 7.2859 (0.5); 7.2843 (0.6); 7.2827 (0.6); 7.2813 (0.5);
7.2620 (14.8); 7.1077 (0.6); 7.1045 (0.6); 7.0869 (0.6); 7.0831 (1.0); 7.0794 (0.6); 7.0618 (0.5); 7.0586 (0.5); 5.3004 (4.8);
4.1309 (0.8); 4.1131 (0.8); 3.7682 (7.8); 3.5761 (16.0); 3.5562 (0.9); 3.5409 (2.2); 3.5256 (2.2); 3.5101 (0.9); 2.5218 (1.7);
2.5063 (2.7); 2.4911 (1.6); 2.0454 (3.8); 1.2774 (1.1); 1.2596 (2.3); 1.2418 (1.1); 0.0080 (0.6); −0.0002 (22.3); −0.0085 (0.6)
VII-011: ¹H-NMR (400.6 MHz, CDCl3):
δ = 7.4393 (0.7); 7.4351 (0.8); 7.4207 (0.6); 7.4162 (0.6); 7.4025 (0.5); 7.3879 (0.6); 7.3835 (0.5); 7.2620 (8.0); 7.2447 (0.7);
7.2255 (1.1); 7.1314 (0.6); 7.1259 (1.0); 7.1220 (0.6); 7.1185 (0.5); 7.1127 (0.6); 7.1103 (0.7); 7.1050 (1.3); 7.1015 (1.0);
7.0914 (0.6); 7.0858 (0.6); 7.0801 (1.1); 7.0619 (0.5); 7.0581 (0.8); 7.0546 (0.5); 6.9874 (0.6); 6.9830 (0.5); 5.3003 (4.2);
4.2298 (1.5); 4.1623 (5.6); 4.1181 (7.0); 3.9701 (1.7); 3.7315 (16.0); 3.1842 (11.4); 3.0133 (2.7); 1.5743 (12.3); 1.2593 (0.7);
1.2563 (0.7); −0.0002 (10.7)

The present invention further provides for the use of one or more compounds of the general formula (I) and/or salts thereof, as defined above, preferably in one of the embodiments identified as preferred or particularly preferred, in particular one or more compounds of the formulae (I.001) to (VII.002) and/or salts thereof, in each case as defined above,

as herbicide and/or plant growth regulator, preferably in crops of useful plants and/or ornamental plants.

The present invention further provides a method of controlling harmful plants and/or for regulating the growth of plants, characterized in that an effective amount
of one or more compounds of the general formula (I) and/or salts thereof, as defined above, preferably in one of the embodiments identified as preferred or particularly preferred, in particular one or more compounds of the formulae (I.001) to (VII.002) and/or salts thereof, in each case as defined above, or
of a composition of the invention, as defined below,
   is applied to the (harmful) plants, seeds of (harmful) plants, the soil in which or on which the (harmful) plants grow or the area under cultivation.

The present invention also provides a method for controlling unwanted plants, preferably in crops of useful plants, characterized in that an effective amount
of one or more compounds of the general formula (I) and/or salts thereof, as defined above, preferably in one of the embodiments identified as preferred or particularly preferred, in particular one or more compounds of the formulae (I.001) to (VII.002) and/or salts thereof, in each case as defined above, or
of a composition of the invention, as defined below,
   is applied to unwanted plants (for example harmful plants such as mono- or dicotyledonous weeds or unwanted crop plants), the seed of the unwanted plants (i.e. plant seeds, for example grains, seeds or vegetative propagation organs such as tubers or shoot parts with buds), the soil in which or on which the unwanted plants grow (for example the soil of crop land or non-crop land) or the area under cultivation (i.e. the area on which the unwanted plants will grow).

The present invention also further provides methods for controlling for regulating the growth of plants, preferably of useful plants, characterized in that an effective amount
of one or more compounds of the general formula (I) and/or salts thereof, as defined above, preferably in one of the embodiments identified as preferred or particularly preferred, in particular one or more compounds of the general formulae (I.001) to (VII.002) and/or salts thereof, in each case as defined above, or
of a composition of the invention, as defined below,
   is applied to the plant, the seed of the plant (i.e. plant seed, for example grains, seeds or vegetative propagation organs such as tubers or shoot parts with buds), the soil in which or on which the plants grow (for example the soil of crop land or non-crop land) or the area under cultivation (i.e. the area on which the plants will grow).

In this context, the compounds of the invention or the compositions of the invention can be deployed, for example, by pre-sowing (if appropriate also by incorporation into the soil), pre-emergence and/or post-emergence methods. Specific examples of some representatives of the monocotyledonous and dicotyledonous weed flora which can be controlled by the compounds of the invention are as follows, though there is no intention to restrict the enumeration to particular species.

In a method of the invention for controlling harmful plants or for regulating the growth of plants, preference is given to using one or more compounds of the general formula (I) and/or salts thereof for control of harmful plants or for regulation of growth in crops of useful plants or ornamental plants, where the useful plants or ornamental plants in a preferred configuration are transgenic plants.

The inventive compounds of the general formula (I) and/or their salts are suitable for controlling the following genera of monocotyledonous and dicotyledonous harmful plants:

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous harmful plants of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the compounds of the invention are applied to the soil surface before germination of the harmful plants (weed grasses and/or broad-leaved weeds) (pre-emergence method), either the seedlings of the weed grasses or broad-leaved weeds are prevented completely from emerging or they grow until they have reached the cotyledon stage, but then stop growing and eventually, after three to four weeks have elapsed, die completely.

If the active ingredients are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

Although the compounds of the invention display excellent herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Miscanthus, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, are damaged only to an insignificant extent, or not at all, depending on the structure of the respective compound of the invention and its application rate. For these reasons, the present compounds are very suitable for selective control of unwanted plant growth in plant crops such as agriculturally useful plants or ornamental plants.

In addition, the compounds of the invention (depending on their particular structure and the application rate deployed) have outstanding growth-regulating properties in crop plants. They intervene in the plants' own metabolism with regulatory effect, and can thus be used for the controlled influencing of plant constituents and to facilitate harvesting, for example by triggering desiccation and stunted growth. Furthermore, they are also suitable for the general control and inhibition of unwanted vegetative growth without killing the plants in the process. Inhibition of vegetative growth plays a major role for many mono- and dicotyledonous crops since, for example, this can reduce or completely prevent lodging.

By virtue of their herbicidal and plant growth regulatory properties, the active ingredients can also be used to control harmful plants in crops of genetically modified plants or plants modified by conventional mutagenesis. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses.

Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material.

It is preferred with a view to transgenic crops to use the compounds of the invention and/or their salts in economically important transgenic crops of useful plants and ornamentals, for example of cereals such as wheat, barley, rye, oats, millet, rice and corn or else crops of sugar beet, cotton, soybean, oilseed rape, potato, tomato, peas and other vegetables.

It is also preferred to employ the compounds of the invention as herbicides in crops of useful plants which are resistant, or have been made resistant by recombinant means, to the phytotoxic effects of the herbicides.

By virtue of their herbicidal and plant growth regulatory properties, the active ingredients can also be used to control harmful plants in crops of genetically modified plants which are known or are yet to be developed. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further special properties may be tolerance or resistance to abiotic stressors, for example heat, cold, drought, salinity and ultraviolet radiation.

Preference is given to the use of the inventive compounds of the general formula (I) or salts thereof in economically important transgenic crops of useful plants and ornamentals, for example of cereals such as wheat, barley, rye, oats, triticale, millet, rice, cassava and corn, or else crops of sugar beet, cotton, soybean, oilseed rape, potatoes, tomatoes, peas and other vegetables.

It is preferable to employ the compounds of the general formula (I) as herbicides in crops of useful plants which are resistant, or have been made resistant by recombinant means, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods.

A large number of molecular-biological techniques by means of which novel transgenic plants with modified properties can be generated are known to the person skilled in the art. For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. To connect the DNA fragments to each other, adapters or linkers may be added to the fragments.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product.

To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Sequences of this kind are known to the person skilled in the art (see, for example, Braun et al., EMBO J. 11 (1992), 3219-3227). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Obtainable in this way are transgenic plants having properties altered by overexpression, suppression or inhibition of homologous (=natural) genes or gene sequences or expression of heterologous (=foreign) genes or gene sequences.

It is preferable to employ the inventive compounds (I) in transgenic crops which are resistant to growth regulators such as, for example, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, glyphosates, glufosinates or benzoylisoxazoles and analogous active ingredients.

When the active ingredients of the invention are employed in transgenic crops, not only do the effects towards harmful plants observed in other crops occur, but frequently also effects which are specific to the application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the inventive compounds of the general formula (I) and/or their salts as herbicides for controlling harmful plants in crops of useful plants or ornamentals, optionally in transgenic crop plants.

Preference is given to the use in cereals, here preferably corn, wheat, barley, rye, oats, millet or rice, by the pre- or post-emergence method.

Preference is also given to the use in soybeans by the pre- or post-emergence method.

The inventive use for control of harmful plants or for regulation of plant growth also includes the case in which the active ingredient of the general formula (I) or its salt is not formed from a precursor substance (“prodrug”) until after application on the plant, in the plant or in the soil.

The invention also provides for the use of one or more compounds of the general formula (I) or salts thereof or of a composition of the invention (as defined below) (in a method) for controlling harmful plants or for regulating the growth of plants which comprises applying an effective amount of one or more compounds of the general formula (I) or salts thereof onto the plants (harmful plants, if appropriate together with the useful plants), plant seeds, the soil in which or on which the plants grow or the area under cultivation.

The invention also provides a herbicidal and/or plant growth-regulating composition, characterized in that the composition comprises

(a) one or more compounds of the general formula (I) and/or salts thereof, as defined above, preferably in one of the embodiments identified as preferred or particularly preferred, in particular one or more compounds of the formulae (I.001) to (VII.002) and/or salts thereof, in each case as defined above,

and

(b) one or more further substances selected from groups (i) and/or (ii):

(i) one or more further agrochemically active substances, preferably selected from the group consisting of insecticides, acaricides, nematicides, further herbicides (i.e. those not conforming to the general formula (I) defined above), fungicides, safeners, fertilizers and/or further growth regulators,

(ii) one or more formulation auxiliaries customary in crop protection.

The further agrochemically active substances of component (i) of a composition of the invention are preferably selected from the group of substances mentioned in “The Pesticide Manual”, 16th edition, The British Crop Protection Council and the Royal Soc. of Chemistry, 2012.

A herbicidal or plant growth-regulating composition of the invention comprises preferably one, two, three or more formulation auxiliaries (ii) customary in crop protection selected from the group consisting of surfactants, emulsifiers, dispersants, film formers, thickeners, inorganic salts, dusting agents, carriers that are solid at 25° C. and 1013 mbar, preferably adsorptive granulated inert materials, wetting agents, antioxidants, stabilizers, buffer substances, antifoam agents, water, organic solvents, preferably organic solvents miscible with water in any ratio at 25° C. and 1013 mbar.

The inventive compounds of the general formula (I) can be used in the form of wettable powders, emulsifiable concentrates, sprayable solutions, dusting products or granules in the customary formulations. The invention therefore also provides herbicidal and plant growth-regulating compositions which comprise compounds of the general formula (I) and/or salts thereof.

The compounds of the general formula (I) and/or salts thereof can be formulated in various ways according to which biological and/or physicochemical parameters are required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes.

These individual formulation types and the formulation auxiliaries, such as inert materials, surfactants, solvents and further additives, are known to the person skilled in the art and are described, for example, in: Watkins, “Handbook of Insecticide Dust Diluents and Carriers”, 2nd ed., Darland Books, Caldwell N.J., H. v. Olphen, “Introduction to Clay Colloid Chemistry”, 2nd ed., J. Wiley & Sons, N.Y., C. Marsden, “Solvents Guide”, 2nd ed., Interscience, N.Y. 1963, McCutcheon's “Detergents and Emulsifiers Annual”, MC Publ. Corp., Ridgewood N.J., Sisley and Wood, “Encyclopedia of Surface Active Agents”, Chem. Publ. Co. Inc., N.Y. 1964, Schönfeldt, “Grenzflächenaktive Äthylenoxidaddukte” [Interface-active Ethylene Oxide Adducts], Wiss. Verlagsgesellschaft, Stuttgart 1976, Winnacker-Küchler, “Chemische Technologie”, Volume 7, C. Hanser Verlag Munich, 4th ed. 1986.

Wettable powders are preparations which can be dispersed uniformly in water and, in addition to the active ingredient, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the active herbicidal ingredients are finely ground, for example in customary apparatuses such as hammer mills, blower mills and air-jet mills, and simultaneously or subsequently mixed with the formulation auxiliaries.

Emulsifiable concentrates are produced by dissolving the active ingredient in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonate salts, for example calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active ingredient with finely distributed solids, for example talc, natural clays, such as kaolin, bentonite and pyrophyllite, or diatomaceous earth.

Suspension concentrates may be water- or oil-based. They may be produced, for example, by wet-grinding by means of commercial bead mills and optional addition of surfactants as already listed above, for example, for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be produced either by spraying the active ingredient onto granular inert material capable of adsorption or by applying active ingredient concentrates to the surface of carrier substances, such as sand, kaolinites or granular inert material, by means of adhesives, for example polyvinyl alcohol, sodium polyacrylate or else mineral oils. Suitable active ingredients can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan granules, fluidized bed granules, extruder granules and spray granules, see, for example, processes in “Spray-Drying Handbook” 3rd ed. 1979, G. Goodwin Ltd., London; J. E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations, preferably herbicidal or plant growth-regulating compositions, of the present invention preferably comprise a total amount of 0.1 to 99% by weight, preferably 0.5 to 95% by weight, more preferably 1 to 90% by weight, especially preferably 2 to 80% by weight, of active ingredients of the general formula (I) and their salts.

In wettable powders, the active ingredient concentration is, for example, about 10% to 90% by weight, the remainder to 100% by weight consisting of customary formulation constituents. In emulsifiable concentrates, the active ingredient concentration may be about 1% to 90% and preferably 5% to 80% by weight. Formulations in the form of dusts comprise 1% to 30% by weight of active ingredient, preferably usually 5% to 20% by weight of active ingredient; sprayable solutions contain about 0.05% to 80% by weight, preferably 2% to 50% by weight of active ingredient. In the case of water-dispersible granules, the active ingredient content depends partly on whether the active compound is present in liquid or solid form and on which granulation auxiliaries, fillers, and so forth are used. In the water-dispersible granules, the content of active ingredient is, for example, between 1% and 95% by weight, preferably between 10% and 80% by weight.

In addition, the active ingredient formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity.

Examples of formulation auxiliaries are described, inter alia, in “Chemistry and Technology of Agrochemical Formulations”, ed. D. A. Knowles, Kluwer Academic Publishers (1998).

The compounds of the general formula (I) or salts thereof can be used as such or in the form of their preparations (formulations) in a combination with other pesticidally active substances, for example insecticides, acaricides, nematicides, herbicides, fungicides, safeners, fertilizers and/or growth regulators, for example in the form of a finished formulation or of a tank mix. The combination formulations can be produced on the basis of the abovementioned formulations, taking account of the physical properties and stabilities of the active ingredients to be combined.

Combination partners usable for the inventive compounds of the general formula (I) in mixed formulations or in a tankmix are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II, protoporphyrinogen oxidase, as described, for example, in Weed Research 26 (1986) 441-445 or “The Pesticide Manual”, 16th edition, The British Crop Protection Council and the Royal Soc. of Chemistry, 2012, and the literature cited therein.

Of particular interest is the selective control of harmful plants in crops of useful plants and ornamentals. Although the inventive compounds of the general formula (I) have already demonstrated very good to adequate selectivity in a large number of crops, in principle, in some crops and in particular also in the case of mixtures with other, less selective herbicides, phytotoxicities on the crop plants may occur. In this connection, combinations of inventive compounds (I) that are of particular interest are those which comprise the compounds of the general formula (I) or their combinations with other herbicides or pesticides and safeners. The safeners, which are used in an antidotically effective amount, reduce the phytotoxic side effects of the herbicides/pesticides employed, for example in economically important crops, such as cereals (wheat, barley, rye, corn, rice, millet), sugarbeet, sugarcane, oilseed rape, cotton and soybeans, preferably cereals.

The weight ratios of herbicide (mixture) to safener depend generally on the herbicide application rate and the efficacy of the safener in question and may vary within wide limits, for example in the range from 200:1 to 1:200, preferably 100:1 to 1:100, in particular 20:1 to 1:20. Analogously to the compounds of the general formula (I) or mixtures thereof, the safeners can be formulated with further herbicides/pesticides and be provided and employed as a finished formulation or tank mix with the herbicides.

For application, the herbicide formulations or herbicide-safener formulations in the commercial form are diluted if appropriate in a customary manner, for example with water in the case of wettable powders, emulsifiable concentrates, dispersions and water-dispersible granules. Preparations in dust form, granules for soil application or granules for scattering and sprayable solutions are not normally diluted further with other inert substances prior to application.

The application rate of the compounds of the general formula (I) and/or their salts is affected to a certain extent by external conditions such as temperature, humidity, etc. The application rate may vary within wide limits. For the application as a herbicide for controlling harmful plants, the total amount of compounds of the general formula (I) and their salts is preferably in the range from 0.001 to 10.0 kg/ha, with preference in the range from 0.005 to 5 kg/ha, more preferably in the range from 0.01 to 1.5 kg/ha, particularly preferably in the range from 0.05 to 1 kg/ha. This applies both to pre-emergence and to post-emergence application.

When compounds of the general formula (I) and/or their salts are used as plant growth regulator, for example as culm stabilizer for crop plants like those mentioned above, preferably cereal plants, such as wheat, barley, rye, triticale, millet, rice or corn, the total application rate is preferably in the range of from 0.001 to 2 kg/ha, preferably in the range of from 0.005 to 1 kg/ha, in particular in the range of from 10 to 500 g/ha, very particularly preferably in the range from 20 to 250 g/ha. This applies both to pre-emergence and to post-emergence application.

The application as culm stabilizer may take place at various stages of the growth of the plants. Preferred is, for example, the application after the tillering phase, at the beginning of the longitudinal growth.

As an alternative, application as plant growth regulator is also possible by treating the seed, which includes various techniques for dressing and coating seed. The application rate depends on the particular techniques and can be determined in preliminary tests.

Combination partners usable for the inventive compounds of the general formula (I) in compositions of the invention (e.g. mixed formulations or in a tankmix) are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase, as described, for example, from Weed Research 26 (1986) 441-445 or “The Pesticide Manual”, 16th edition, The British Crop Protection Council and the Royal Soc. of Chemistry, 2012, and literature cited therein. Known herbicides or plant growth regulators which can be combined with the compounds of the invention are, for example, the following, where said active ingredients are referred to either by their “common name” in accordance with the International Organization for Standardization (ISO) or by the chemical name or by the code number. They always encompass all the use forms, for example acids, salts, esters and also all isomeric forms such as stereoisomers and optical isomers, even if they are not mentioned explicitly.

### Examples of Such Herbicidal Mixing Partners are

acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro methylphenyl)-5-fluoropyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamine, -ethyl, 2-ethylhexyl, -isobutyl, -isooctyl, -isopropylammonium, -potassium, -triisopropanolammonium and -trolamine, 2,4-DB, 2,4-DB-butyl, -dimethylammonium, isooctyl, -potassium and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-[2-chloro-4-fluoro-5-[4-(3-fluoropropyl)-4,5-dihydro-5-oxo-1H-tetrazol-1-yl]-phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. 0-(2,4-dimethyl-6-nitrophenyl) O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl(2,4-dichlorophenoxy)acetate, imazamethabenz, Imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPB-methyl, -ethyl and -sodium, mecoprop, mecoprop-sodium, and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinat, monolinuron, monosulfuron, monosulfuron-ester, MT-5950, i.e. N-[3-chloro-4-(1-methylethyl)phenyl]-2-methylpentanamide, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trifluoroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, and the following compounds:

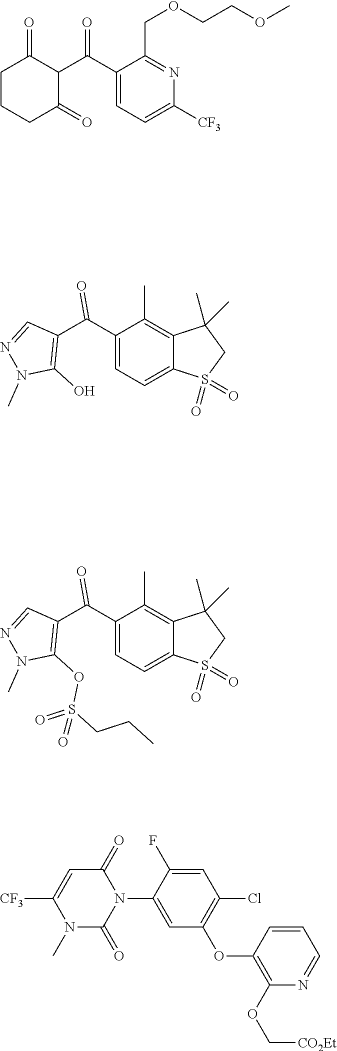 [SMILES: COCCOCc1nc(C(F)(F)F)ccc1C(=O)C1C(=O)CCCC1=O ; Cc1c(C(=O)c2cnn(C)c2O)ccc2c1C(C)(C)CS2(=O)=O ; CCCS(=O)(=O)Oc1c(C(=O)c2ccc3c(c2C)C(C)(C)CS3(=O)=O)cnn1C ; CCOC(=O)COc1ncccc1Oc1cc(-n2c(=O)cc(C(F)(F)F)n(C)c2=O)c(F)cc1Cl]

Examples of plant growth regulators as possible mixing partners are:

acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenolate mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

Useful combination partners for the inventive compounds of the general formula (I) also include, for example, the following safeners:

S1) Compounds from the group of heterocyclic carboxylic acid derivatives:

S1^{a}) Compounds of the dichlorophenylpyrazoline-3-carboxylic acid type (S P), preferably compounds such as

1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;

S1^{b}) Derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl) isopropylpyrazole-3-carboxylate (S1-3), ethyl 1-(2,4-dichlorophenyl)-5-(1,1-dimethylethyl)pyrazole carboxylate (S1-4) and related compounds as described in EP-A-333131 and EP-A-269806;

S1^{c}) Derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described, for example, in EP-A-268554;

S1^{d}) Compounds of the triazolecarboxylic acid type (S1^{d}), preferably compounds such as fenchlorazole(-ethyl ester), i.e. ethyl 1-(2,4-dichlorophenyl)-5-trichloromethyl-1H-1,2,4-triazole-3-carboxylate (S1-7), and related compounds, as described in EP-A-174562 and EP-A-346620;

S1^{e}) Compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (51-9) and related compounds as described in WO-A-91/08202, or 5,5-diphenyl-2-isoxazolinecarboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13) as described in patent application WO-A-95/07897.

S2) Compounds from the group of the 8-quinolinoxy derivatives (S2):

S2^{a}) Compounds of the 8-quinolinoxyacetic acid type (S2^{a}), preferably 1-methylhexyl(5-chloro-8-quinolinoxy)acetate (“cloquintocet-mexyl”) (S2-1), 1,3-dimethylbut-1-yl(5-chloro-8-quinolinoxy)acetate (S2-2), 4-allyloxybutyl(5-chloro-8-quinolinoxy)acetate (S2-3), 1-allyloxyprop-2-yl(5-chloro-8-quinolinoxy)acetate (S2-4), ethyl(5-chloro-8-quinolinoxy)acetate (S2-5),

methyl (5-chloro-8-quinolinoxy)acetate (S2-6),

allyl(5-chloro-8-quinolinoxy)acetate (S2-7), 2-(2-propylideneiminoxy)-1-ethyl(5-chloro-8-quinolinoxy)acetate (S2-8), 2-oxoprop-1-yl(5-chloro-8-quinolinoxy)acetate (S2-9) and related compounds, as described in EP-A-86750, EP-A-94349 and EP-A-191736 or EP-A-0 492 366, and also (5-chloro-8-quinolinoxy)acetic acid (S2-10), hydrates and salts thereof, for example the lithium, sodium, potassium, calcium, magnesium, aluminium, iron, ammonium, quaternary ammonium, sulfonium or phosphonium salts thereof, as described in WO-A-2002/34048;

S2^{b}) Compounds of the (5-chloro-8-quinolinoxy)malonic acid type (S2^{b}), preferably compounds such as diethyl(5-chloro-8-quinolinoxy)malonate, diallyl(5-chloro-8-quinolinoxy)malonate, methyl ethyl(5-chloro-8-quinolinoxy)malonate and related compounds, as described in EP-A-0 582 198.

S3) Active ingredients of the dichloroacetamide type (S3), which are frequently used as pre-emergence safeners (soil-acting safeners), for example

“dichlormid” (N,N-diallyl-2,2-dichloroacetamide) (S3-1),

“R-29148” (3-dichloroacetyl-2,2,5-trimethyl-1,3-oxazolidine) from Stauffer (S3-2),

“R-28725” (3-dichloroacetyl-2,2-dimethyl-1,3-oxazolidine) from Stauffer (S3-3),

“benoxacor” (4-dichloroacetyl-3,4-dihydro-3-methyl-2H-1,4-benzoxazine) (S3-4),

“PPG-1292” (N-allyl-N-[(1,3-dioxolan-2-yl]methylldichloroacetamide) from PPG Industries (S3-5),

“DKA-24” (N-allyl-N-Rallylaminocarbonyl)methyl]dichloroacetamide) from Sagro-Chem (S3-6),

“AD-67” or “MON 4660” (3-dichloroacetyl-1-oxa-3-azaspiro[4.5]decane) from Nitrokemia or Monsanto (S3-7),

“TI-35” (1-dichloroacetylazepane) from TRI-Chemical RT (S3-8), “diclonon” (dicyclonon) or “BAS145138” or “LAB145138” (S3-9)

((RS)-1-dichloroacetyl-3,3,8a-trimethylperhydropyrrolo[1,2-a]pyrimidin-6-one) from BASF,

“furilazole” or “MON 13900” ((RS)-3-dichloroacetyl-5-(2-furyl)-2,2-dimethyloxazolidine) (S3-10), and the (R) isomer thereof (S3-11).

S4) Compounds from the class of the acylsulfonamides (S4):

S4^{a})N-Acylsulfonamides of the formula (S4^{a}) and salts thereof, as described in WO-A-97/45016,

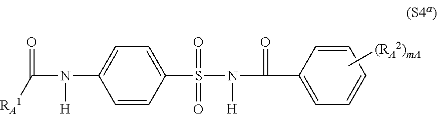

in which

R_{A}¹ is (C₁-C₆)-alkyl, (C₃-C₆)-cycloalkyl, where the 2 latter radicals are substituted by v_{A} substituents from the group of halogen, (C₁-C₄)-alkoxy, (C₁-C₆)-haloalkoxy and (C₁-C₄)-alkylthio and, in the case of cyclic radicals, also by (C₁-C₄)-alkyl and (C₁-C₄)-haloalkyl;

R_{A}² is halogen, (C₁-C₄)-alkyl, (C₁-C₄)-alkoxy, CF₃;

m_{A} is 1 or 2;

V_{A} is 0, 1, 2 or 3;

S4^{b}) Compounds of the 4-(benzoylsulfamoyl)benzamide type of the formula (S4^{b}) and salts thereof, as described in WO-A-99/16744,

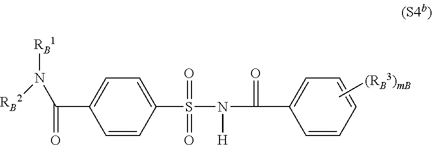

in which

R_{B}¹, R_{B}² are independently hydrogen, (C₁-C₆)-alkyl, (C₃-C₆)-cycloalkyl, (C₃-C₆)-alkenyl, (C₃-C₆)-alkynyl,

R_{B}³ is halogen, (C₁-C₄)-alkyl, (C₁-C₄)-haloalkyl or (C₁-C₄)-alkoxy and

m_{B} is 1 or 2,

e.g. those in which

R_{B}¹=cyclopropyl, R_{B}²=hydrogen and (R_{B}³)=2-OMe (“cyprosulfamide”, S4-1),

R_{B}¹=cyclopropyl, R_{B}²=hydrogen and (R_{B}³)=5-C₁-2-OMe (S4-2),

R_{B}¹=ethyl, R_{B}²=hydrogen and (R_{B}³)=2-OMe (S4-3),

R_{B}¹=isopropyl, R_{B}²=hydrogen and (R_{B}³)=5-C₁-2-OMe (S4-4) and

R_{B}¹=isopropyl, R_{B}²=hydrogen and (R_{B}³)=2-OMe (S4-5);

S4c) Compounds from the class of the benzoylsulfamoylphenylureas of the formula (S4^{c}), as described in EP-A-365484,

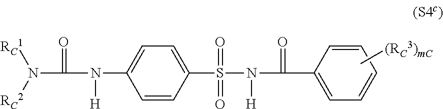

in which

R_{c}¹, R_{c}² are independently hydrogen, (C₃-C₈)-alkyl, (C₃-C₈)-cycloalkyl, (C₃-C₆)-alkenyl, (C₃-C₆)-alkynyl,

R_{c}³ is halogen, (C₁-C₄)-alkyl, (C₁-C₄)-alkoxy, CF₃ and

mc is 1 or 2;

for example

1-[4-(N-2-methoxybenzoylsulfamoyl)phenyl]-3-methylurea,

1-[4-(N-2-methoxybenzoylsulfamoyl)phenyl]-3,3-dimethylurea,

1-[4-(N-4,5-dimethylbenzoylsulfamoyl)phenyl]-3-methylurea;

S4^{d}) Compounds of the N-phenylsulfonylterephthalamide type of the formula (S4^{d}) and salts thereof, which are known, for example, from CN 101838227,

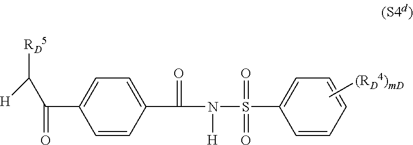

in which

R_{D}⁴ is halogen, (C₁-C₄)-alkyl, (C₁-C₄)-alkoxy, CF₃;

m_{D} is 1 or 2;

R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.

S5) Active ingredients from the class of the hydroxyaromatics and the aromatic-aliphatic carboxylic acid derivatives (S5), for example

ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.

S6) Active ingredients from the class of the 1,2-dihydroquinoxalin-2-ones (S6), for example

1-methyl-3-(2-thienyl)-1,2-dihydroquinoxalin-2-one, 1-methyl-3-(2-thienyl)-1,2-dihydroquinoxaline thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.

S7) Compounds from the class of the diphenylmethoxyacetic acid derivatives (S7), e.g. methyl diphenylmethoxyacetate (CAS Reg. No. 41858-19-9) (S7-1), ethyl diphenylmethoxyacetate or diphenylmethoxyacetic acid, as described in WO-A-98/38856.

S8) Compounds of the formula (S8), as described in WO-A-98/27049,

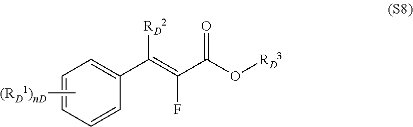

in which the symbols and indices are defined as follows:

R_{D}¹ is halogen, (C₁-C₄)-alkyl, (C₁-C₄)-haloalkyl, (C₁-C₄)-alkoxy, (C₁-C₄)-haloalkoxy,

R_{D}² is hydrogen or (C₁-C₄)-alkyl,

R_{D}³ is hydrogen, (C₁-C₈)-alkyl, (C₂-C₄)-alkenyl, (C₂-C₄)-alkynyl or aryl, where each of the aforementioned carbon-containing radicals is unsubstituted or substituted by one or more, preferably up to three, identical or different radicals from the group consisting of halogen and alkoxy; or salts thereof,

n_{D} is an integer from 0 to 2.

S9) Active ingredients from the class of the 3-(5-tetrazolylcarbonyl)-2-quinolones (S9), for example

1,2-dihydro-4-hydroxy-1-ethyl-3-(5-tetrazolylcarbonyl)-2-quinolone (CAS Reg. No.: 219479-18-2), 1,2-dihydro-4-hydroxy-1-methyl-3-(5-tetrazolylcarbonyl)-2-quinolone (CAS Reg. No. 95855-00-8), as described in WO-A-1999/000020.

S10) Compounds of the formulae (S10a) or (S10^{b})
as described in WO-A-2007/023719 and WO-A-2007/023764
   in which

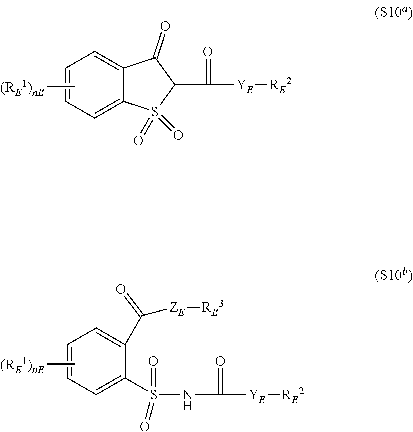

R_{E}¹ is halogen, (C₁-C₄)-alkyl, methoxy, nitro, cyano, CF₃, OCF₃,

Y_{E}, Z_{E} are independently O or S,

n_{E} is an integer from 0 to 4,

R_{E}² is (C₁-C₁₆)-alkyl, (C₂-C₆)-alkenyl, (C₃-C₆)-cycloalkyl, aryl; benzyl, halobenzyl,

R_{E}³ is hydrogen or (C₁-C₆)-alkyl.

S11) Active ingredients of the oxyimino compounds type (S11), which are known as seed-dressing agents, for example

“oxabetrinil” ((Z)-1,3-dioxolan-2-ylmethoxyimino(phenyl)acetonitrile) (S11-1), which is known as a seed-dressing safener for millet/sorghum against metolachlor damage,

“fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against metolachlor damage, and

“cyometrinil” or “CGA-43089” ((Z)-cyanomethoxyimino(phenyl)acetonitrile) (S11-3), which is known as a seed-dressing safener for millet/sorghum against metolachlor damage.

S12) Active ingredients from the class of the isothiochromanones (S12), for example methyl[(3-oxo-1H-2-benzothiopyran-4(3H)-ylidene)methoxy]acetate (CAS Reg. No. 205121-04-6) (512-1) and related compounds from WO-A-1998/13361.

S13) One or more compounds from group (S13):

“naphthalic anhydride” (1,8-naphthalenedicarboxylic anhydride) (S13-1), which is known as a seed-dressing safener for corn against thiocarbamate herbicide damage,

“fenclorim” (4,6-dichloro-2-phenylpyrimidine) (S13-2), which is known as a safener for pretilachlor in sown rice,

“flurazole” (benzyl 2-chloro-4-trifluoromethyl-1,3-thiazole-5-carboxylate) (513-3), which is known as a seed-dressing safener for millet/sorghum against alachlor and metolachlor damage,

“CL 304415” (CAS Reg. No. 31541-57-8)

(4-carboxy-3,4-dihydro-2H-1-benzopyran-4-acetic acid) (513-4) from American Cyanamid, which is known as a safener for corn against damage by imidazolinones,

“MG 191” (CAS Reg. No. 96420-72-3) (2-dichloromethyl-2-methyl-1,3-dioxolane) (513-5) from Nitrokemia, which is known as a safener for corn,

“MG 838” (CAS Reg. No. 133993-74-5)

(2-propenyl 1-oxa-4-azaspiro[4.5]decane-4-carbodithioate) (S13-6) from Nitrokemia

“disulfoton” (0,0-diethyl S-2-ethylthioethyl phosphorodithioate) (S13-7),

“dietholate” (0,0-diethyl 0-phenyl phosphorothioate) (S13-8),

“mephenate” (4-chlorophenyl methylcarbamate) (513-9).

S14) Active ingredients which, in addition to herbicidal action against harmful plants, also have safener action on crop plants such as rice, for example

“dimepiperate” or “MY-93” (S-1-methyl 1-phenylethylpiperidine-1-carbothioate), which is known as a safener for rice against damage by the herbicide molinate,

“daimuron” or “SK 23” (1-(1-methyl-1-phenylethyl)-3-p-tolylurea), which is known as a safener for rice against damage by the herbicide imazosulfuron,

“cumyluron”=“JC-940” (3-(2-chlorophenylmethyl)-1-(1-methyl-1-phenylethyl)urea, see JP-A-60087270), which is known as a safener for rice against damage by some herbicides,

“methoxyphenone” or “NK 049” (3,3′-dimethyl-4-methoxybenzophenone), which is known as a safener for rice against damage by some herbicides,

“CSB” (1-bromo-4-(chloromethylsulfonyl)benzene) from Kumiai, (CAS Reg. No. 54091-06-4), which is known as a safener against damage by some herbicides in rice.

S15) Compounds of the formula (S15) or tautomers thereof

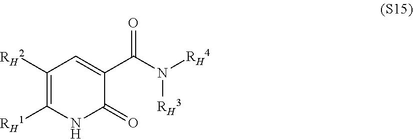

as described in WO-A-2008/131861 and WO-A-2008/131860
in which

R_{H}¹ is a (C₁-C₆)-haloalkyl radical and

R_{H}² is hydrogen or halogen and

R_{H}³, R_{H}⁴ are independently hydrogen, (C₁-C₁₆)-alkyl, (C₂-C₁₆)-alkenyl or (C₂-C₁₆)-alkynyl,

where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,

or (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,

where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄) -haloalkoxy]carbonyl, (C₃-C₆)-cycloalkyl which is unsubstituted or substituted, phenyl which is unsubstituted or substituted, and heterocyclyl which is unsubstituted or substituted,

or

R_{H}³ is (C₁-C₄)-alkoxy, (C₂-C₄)-alkenyloxy, (C₂-C₆)-alkynyloxy or (C₂-C₄)-haloalkoxy and

R_{H}⁴ is hydrogen or (C₁-C₄)-alkyl or

R_{H}³ and R_{H}⁴ together with the directly attached nitrogen atom represent a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.

S16) Active ingredients which are used primarily as herbicides but also have safener action on crop plants, for example

(2,4-dichlorophenoxy)acetic acid (2,4-D),

(4-chlorophenoxy)acetic acid,

(R,S)-2-(4-chloro-o-tolyloxy)propionic acid (mecoprop),

4-(2,4-dichlorophenoxy)butyric acid (2,4-DB),

(4-chloro-o-tolyloxy)acetic acid (MCPA),

4-(4-chloro-o-tolyloxy)butyric acid,

4-(4-chlorophenoxy)butyric acid,

3,6-dichloro-2-methoxybenzoic acid (dicamba),

1-(ethoxycarbonyl)ethyl 3,6-dichloro-2-methoxybenzoate (lactidichloro-ethyl).

Preferred safeners in combination with the compounds of the formula (I) according to the invention and/or salts thereof, in particular with the compounds of the formulae (I.1) to (1.182) and/or salts thereof, are: cloquintocet-mexyl, cyprosulfamide, fenchlorazole ethyl ester, isoxadifen-ethyl, mefenpyr-diethyl, fenclorim, cumyluron, S4-1 and S4-5, and particularly preferred safeners are: cloquintocet-mexyl, cyprosulfamide, isoxadifen-ethyl and mefenpyr-diethyl.

### Biological Examples

1. Pre-emergence herbicidal effect and crop plant compatibility

Seeds of monocotyledonous and dicotyledonous weed plants and crop plants are laid out in sandy loam soil in wood-fibre pots and covered with soil. The compounds of the invention, formulated in the form of wettable powders (WP) or as emulsion concentrates (EC), are then applied to the surface of the covering soil as aqueous suspension or emulsion at a water application rate equating to 600 to 800 L/ha with addition of 0.2% wetting agent.

After the treatment, the pots are placed in a greenhouse and kept under good growth conditions for the trial plants. The damage to the test plants is scored visually after a test period of 3 weeks by comparison with untreated controls (herbicidal activity in percent (%): 100% activity=the plants have died, 0% activity=like control plants).

Undesired Plants/Weeds:

| | | | |
|---|---|---|---|
| ALOMY: | Alopecurus myosuroides | SETVI: | Setaria viridis |
| AMARE: | Amaranthus retroflexus | KCHSC: | Bassia scoparia |
| CYPES: | Cyperus esculentus | ECHCG: | Echinochloa crus-galli |
| LOLRI: | Lolium rigidum | STEME: | Stellaria media |
| VERPE: | Veronica persica | MATIN: | Tripleurospermum inodorum |
| POAAN: | Poa annua | ABUTH: | Abutylon threophrasti |
| DIGSA: | Digitaria sanguinalis | | |

1. Pre-Emergence Effectiveness

As shown by the results from Tables 1.1 to 1.10, compounds of the invention have good herbicidal pre-emergence efficacy against a broad spectrum of weed grasses and broad-leaved weeds.

TABLE 1
Pre-emergence action against ALOMY

| Example | Dosage | ALOMY |
|---|---|---|
| number | [g/ha] | [%] |
| I-002 | 1280 | 90 |
| I-005 | 1280 | 90 |
| I-009 | 1280 | 90 |
| II-001 | 1280 | 100 |
| I-004 | 1280 | 90 |
| I-006 | 1280 | 90 |
| I-010 | 1280 | 100 |
| I-001 | 1280 | 100 |
| II-005 | 1280 | 100 |
| II-007 | 1280 | 100 |
| II-011 | 1280 | 100 |
| II-008 | 1280 | 100 |
| I-013 | 1280 | 100 |
| II-013 | 1280 | 100 |
| I-015 | 1280 | 100 |
| I-022 | 1280 | 90 |
| I-018 | 1280 | 90 |
| I-016 | 1280 | 90 |
| I-020 | 1280 | 90 |
| I-019 | 1280 | 90 |
| I-023 | 1280 | 90 |
| I-024 | 1280 | 90 |
| II-012 | 1280 | 90 |
| II-016 | 1280 | 90 |
| I-014 | 1280 | 100 |
| VII-001 | 1280 | 90 |
| VII-005 | 1280 | 90 |
| II-017 | 1280 | 90 |
| VII-011 | 1280 | 90 |
| VII-007 | 1280 | 90 |
| VII-006 | 1280 | 90 |
| VII-010 | 1280 | 90 |
| VII-009 | 1280 | 90 |

TABLE 2
Pre-emergence action against DIGSA

| Example | Dosage | DIGSA |
|---|---|---|
| number | [g/ha] | [%] |
| I-002 | 1280 | 90 |
| I-005 | 1280 | 100 |
| V-001 | 1280 | 90 |
| I-009 | 1280 | 100 |
| II-001 | 1280 | 100 |
| I-004 | 1280 | 100 |
| I-006 | 1280 | 100 |
| I-010 | 1280 | 100 |
| III-002 | 1280 | 100 |
| I-001 | 1280 | 100 |
| II-005 | 1280 | 100 |
| I-012 | 1280 | 100 |
| II-007 | 1280 | 100 |
| II-011 | 1280 | 100 |
| II-008 | 1280 | 100 |
| I-013 | 1280 | 100 |
| II-013 | 1280 | 100 |
| I-015 | 1280 | 100 |
| I-022 | 1280 | 100 |
| I-018 | 1280 | 100 |
| VI-001 | 1280 | 90 |
| I-016 | 1280 | 100 |
| I-020 | 1280 | 100 |
| I-019 | 1280 | 100 |
| I-023 | 1280 | 100 |
| I-024 | 1280 | 100 |
| II-016 | 1280 | 100 |
| I-027 | 1280 | 100 |
| I-014 | 1280 | 100 |
| VII-001 | 1280 | 100 |

TABLE 3
Pre-emergence action against ECHCG

| Example | Dosage | ECHCG |
|---|---|---|
| number | [g/ha] | [%] |
| I-009 | 1280 | 90 |
| II-001 | 1280 | 100 |
| I-004 | 1280 | 90 |
| I-006 | 1280 | 90 |
| I-010 | 1280 | 100 |
| III-002 | 1280 | 100 |
| II-005 | 1280 | 100 |
| I-012 | 1280 | 100 |
| II-007 | 1280 | 100 |
| II-011 | 1280 | 100 |
| II-008 | 1280 | 100 |
| I-013 | 1280 | 100 |
| II-013 | 1280 | 100 |
| I-015 | 1280 | 100 |
| I-022 | 1280 | 90 |
| I-018 | 1280 | 100 |
| I-016 | 1280 | 100 |
| I-019 | 1280 | 100 |
| I-024 | 1280 | 90 |
| II-012 | 1280 | 90 |
| II-016 | 1280 | 100 |
| I-014 | 1280 | 90 |
| VII-002 | 1280 | 100 |
| VII-003 | 1280 | 100 |
| VII-005 | 1280 | 90 |
| III-005 | 1280 | 90 |
| II-017 | 1280 | 90 |
| VII-007 | 1280 | 100 |
| VII-006 | 1280 | 90 |
| VI-002 | 1280 | 90 |
| VII-010 | 1280 | 90 |
| VII-009 | 1280 | 90 |

TABLE 4
Pre-emergence action against LOLRI

| Example | Dosage | LOLRI |
|---|---|---|
| number | [g/ha] | [%] |
| I-002 | 1280 | 100 |
| I-005 | 1280 | 100 |
| IV-001 | 1280 | 90 |
| I-009 | 1280 | 100 |
| II-001 | 1280 | 100 |
| I-004 | 1280 | 100 |
| I-006 | 1280 | 90 |
| I-010 | 1280 | 100 |
| I-001 | 1280 | 100 |
| II-005 | 1280 | 100 |
| I-012 | 1280 | 90 |
| II-007 | 1280 | 100 |
| II-011 | 1280 | 100 |
| II-008 | 1280 | 100 |
| I-013 | 1280 | 100 |
| II-013 | 1280 | 100 |
| I-015 | 1280 | 100 |
| I-022 | 1280 | 100 |
| I-018 | 1280 | 100 |
| I-016 | 1280 | 90 |
| I-020 | 1280 | 100 |
| I-024 | 1280 | 100 |
| II-012 | 1280 | 100 |
| II-016 | 1280 | 100 |
| I-027 | 1280 | 90 |
| I-014 | 1280 | 100 |
| VII-001 | 1280 | 100 |
| VII-003 | 1280 | 100 |
| VII-005 | 1280 | 90 |
| VII-010 | 1280 | 100 |
| VII-009 | 1280 | 100 |

TABLE 5
Pre-emergence action against POAAN

| Example | Dosage | POAAN |
|---|---|---|
| number | [g/ha] | [%] |
| I-002 | 1280 | 100 |
| I-005 | 1280 | 100 |
| V-001 | 1280 | 90 |
| IV-001 | 1280 | 90 |
| I-009 | 1280 | 100 |
| V-002 | 1280 | 100 |
| II-001 | 1280 | 100 |
| I-004 | 1280 | 100 |
| I-006 | 1280 | 100 |
| I-010 | 1280 | 100 |
| III-002 | 1280 | 100 |
| I-001 | 1280 | 100 |
| II-005 | 1280 | 100 |
| I-012 | 1280 | 100 |
| II-007 | 1280 | 100 |
| II-011 | 1280 | 100 |
| II-008 | 1280 | 100 |
| I-013 | 1280 | 100 |
| II-013 | 1280 | 100 |
| I-015 | 1280 | 100 |
| I-022 | 1280 | 90 |
| I-018 | 1280 | 100 |
| VI-001 | 1280 | 100 |
| I-016 | 1280 | 100 |
| I-020 | 1280 | 100 |
| I-019 | 1280 | 100 |
| I-023 | 1280 | 100 |
| I-024 | 1280 | 100 |
| II-012 | 1280 | 100 |
| II-015 | 1280 | 90 |
| II-016 | 1280 | 100 |
| I-027 | 1280 | 100 |
| I-014 | 1280 | 100 |
| VII-001 | 1280 | 100 |
| VII-003 | 1280 | 100 |
| VII-004 | 1280 | 100 |
| VII-005 | 1280 | 100 |
| III-005 | 1280 | 100 |
| II-017 | 1280 | 100 |
| VII-011 | 1280 | 100 |
| VII-008 | 1280 | 90 |
| VII-007 | 1280 | 100 |
| VII-006 | 1280 | 100 |
| VI-002 | 1280 | 100 |
| VII-010 | 1280 | 100 |
| VII-009 | 1280 | 100 |

TABLE 6
Pre-emergence action against SETVI

| Example | Dosage | SETVI |
|---|---|---|
| number | [g/ha] | [%] |
| I-002 | 1280 | 100 |
| I-005 | 1280 | 100 |
| I-009 | 1280 | 100 |
| V-002 | 1280 | 100 |
| II-001 | 1280 | 100 |
| I-004 | 1280 | 100 |
| I-006 | 1280 | 100 |
| I-010 | 1280 | 100 |
| III-002 | 1280 | 100 |
| I-001 | 1280 | 100 |
| II-005 | 1280 | 100 |
| I-012 | 1280 | 100 |
| II-007 | 1280 | 100 |
| II-011 | 1280 | 100 |
| II-008 | 1280 | 100 |
| I-013 | 1280 | 100 |
| II-013 | 1280 | 100 |
| I-015 | 1280 | 100 |
| I-022 | 1280 | 100 |
| I-018 | 1280 | 100 |
| I-016 | 1280 | 100 |
| I-020 | 1280 | 100 |
| I-019 | 1280 | 100 |
| I-023 | 1280 | 90 |
| I-024 | 1280 | 100 |
| II-012 | 1280 | 90 |
| II-016 | 1280 | 100 |
| I-027 | 1280 | 100 |
| I-014 | 1280 | 100 |
| VII-001 | 1280 | 100 |
| VII-002 | 1280 | 100 |
| VII-003 | 1280 | 100 |
| VII-005 | 1280 | 90 |
| III-005 | 1280 | 90 |
| II-017 | 1280 | 90 |
| VII-007 | 1280 | 100 |
| VII-006 | 1280 | 90 |
| VI-002 | 1280 | 90 |
| VII-010 | 1280 | 100 |
| VII-009 | 1280 | 100 |

TABLE 7
Pre-emergence action against ABUTH

| Example | Dosage | ABUTH |
|---|---|---|
| number | [g/ha] | [%] |
| II-005 | 1280 | 90 |
| II-007 | 1280 | 90 |
| II-011 | 1280 | 90 |
| II-013 | 1280 | 90 |
| I-019 | 1280 | 90 |
| I-014 | 1280 | 100 |
| VII-009 | 1280 | 90 |

TABLE 8
Pre-emergence action against AMARE

| Example | Dosage | AMARE |
|---|---|---|
| number | [g/ha] | [%] |
| I-001 | 1280 | 100 |
| I-002 | 1280 | 100 |
| I-004 | 1280 | 100 |
| I-005 | 1280 | 100 |
| I-006 | 1280 | 100 |
| I-009 | 1280 | 100 |
| I-010 | 1280 | 100 |
| I-012 | 1280 | 100 |
| I-013 | 1280 | 100 |
| I-022 | 1280 | 90 |
| II-001 | 1280 | 100 |
| II-005 | 1280 | 100 |
| II-007 | 1280 | 100 |
| II-008 | 1280 | 100 |
| II-011 | 1280 | 100 |
| II-013 | 1280 | 100 |
| III-002 | 1280 | 100 |
| IV-001 | 1280 | 100 |
| V-001 | 1280 | 100 |
| V-002 | 1280 | 100 |

TABLE 9
Pre-emergence action against KCHSC

| Example | Dosage | KCHSC |
|---|---|---|
| number | [g/ha] | [%] |
| II-005 | 1280 | 90 |
| II-007 | 1280 | 100 |
| II-011 | 1280 | 100 |
| I-013 | 1280 | 90 |
| II-013 | 1280 | 90 |
| I-015 | 1280 | 100 |
| I-016 | 1280 | 100 |
| I-019 | 1280 | 100 |
| I-014 | 1280 | 90 |
| VII-002 | 1280 | 90 |
| VII-007 | 1280 | 90 |
| VII-010 | 1280 | 90 |

TABLE 10
Pre-emergence action against MATIN

| Example | Dosage | MATIN |
|---|---|---|
| number | [g/ha] | [%] |
| I-002 | 1280 | 100 |
| I-005 | 1280 | 100 |
| V-001 | 1280 | 90 |
| IV-001 | 1280 | 90 |
| I-009 | 1280 | 100 |
| V-002 | 1280 | 100 |
| II-001 | 1280 | 100 |
| I-004 | 1280 | 100 |
| I-006 | 1280 | 100 |
| I-010 | 1280 | 100 |
| III-002 | 1280 | 100 |
| I-001 | 1280 | 90 |
| II-005 | 1280 | 100 |
| I-012 | 1280 | 100 |
| II-007 | 1280 | 100 |
| II-011 | 1280 | 100 |
| II-008 | 1280 | 100 |
| I-013 | 1280 | 100 |
| II-013 | 1280 | 100 |
| I-015 | 1280 | 100 |
| I-022 | 1280 | 90 |
| I-018 | 1280 | 100 |
| VI-001 | 1280 | 90 |
| I-016 | 1280 | 100 |
| I-020 | 1280 | 100 |
| I-019 | 1280 | 100 |
| I-023 | 1280 | 100 |
| I-024 | 1280 | 100 |
| II-012 | 1280 | 100 |
| II-015 | 1280 | 100 |
| II-016 | 1280 | 100 |
| I-027 | 1280 | 100 |
| I-026 | 1280 | 100 |
| I-014 | 1280 | 100 |
| VII-001 | 1280 | 100 |
| VII-003 | 1280 | 100 |
| VII-005 | 1280 | 100 |
| III-005 | 1280 | 90 |
| II-017 | 1280 | 90 |
| VII-011 | 1280 | 90 |
| VII-007 | 1280 | 90 |
| VII-006 | 1280 | 90 |
| VI-002 | 1280 | 90 |
| VII-010 | 1280 | 100 |
| VII-009 | 1280 | 100 |

TABLE 11
Pre-emergence action against STEME

| Example | Dosage | STEME |
|---|---|---|
| number | [g/ha] | [%] |
| I-002 | 1280 | 100 |
| I-005 | 1280 | 100 |
| IV-001 | 1280 | 100 |
| I-009 | 1280 | 100 |
| II-001 | 1280 | 90 |
| I-004 | 1280 | 100 |
| I-006 | 1280 | 90 |
| I-010 | 1280 | 100 |
| III-002 | 1280 | 100 |
| I-001 | 1280 | 100 |
| II-005 | 1280 | 100 |
| I-012 | 1280 | 100 |
| II-007 | 1280 | 100 |
| II-011 | 1280 | 100 |
| II-008 | 1280 | 100 |
| I-013 | 1280 | 100 |
| II-013 | 1280 | 100 |
| I-015 | 1280 | 100 |
| I-022 | 1280 | 100 |
| I-018 | 1280 | 100 |
| VI-001 | 1280 | 90 |
| I-016 | 1280 | 100 |
| I-020 | 1280 | 100 |
| I-019 | 1280 | 90 |
| I-023 | 1280 | 100 |
| I-024 | 1280 | 100 |
| II-012 | 1280 | 100 |
| II-015 | 1280 | 100 |
| II-016 | 1280 | 100 |
| I-027 | 1280 | 100 |
| I-014 | 1280 | 100 |
| VII-001 | 1280 | 100 |
| VII-002 | 1280 | 100 |
| VII-003 | 1280 | 100 |
| VII-004 | 1280 | 100 |
| VII-005 | 1280 | 100 |
| III-005 | 1280 | 90 |
| II-017 | 1280 | 90 |
| VII-007 | 1280 | 90 |
| VII-006 | 1280 | 90 |
| VI-002 | 1280 | 90 |
| VII-010 | 1280 | 90 |
| VII-009 | 1280 | 100 |

TABLE 12
Pre-emergence action against VERPE

| Example | Dosage | VERPE |
|---|---|---|
| number | [g/ha] | [%] |
| I-002 | 1280 | 100 |
| I-005 | 1280 | 100 |
| IV-001 | 1280 | 90 |
| I-009 | 1280 | 90 |
| II-001 | 1280 | 100 |
| I-004 | 1280 | 100 |
| I-006 | 1280 | 100 |
| I-010 | 1280 | 100 |
| III-002 | 1280 | 100 |
| I-001 | 1280 | 100 |
| II-005 | 1280 | 100 |
| I-012 | 1280 | 100 |
| II-007 | 1280 | 100 |
| II-011 | 1280 | 100 |
| II-008 | 1280 | 100 |
| I-013 | 1280 | 100 |
| II-013 | 1280 | 100 |
| I-015 | 1280 | 100 |
| I-022 | 1280 | 100 |
| I-018 | 1280 | 100 |
| I-016 | 1280 | 100 |
| I-020 | 1280 | 100 |
| I-019 | 1280 | 100 |
| I-023 | 1280 | 100 |
| I-024 | 1280 | 100 |
| II-012 | 1280 | 100 |
| II-016 | 1280 | 90 |
| I-027 | 1280 | 90 |
| I-014 | 1280 | 100 |
| VII-001 | 1280 | 100 |
| VII-002 | 1280 | 90 |
| VII-003 | 1280 | 100 |
| VII-005 | 1280 | 100 |
| III-005 | 1280 | 90 |
| II-017 | 1280 | 100 |
| VII-011 | 1280 | 90 |
| VII-007 | 1280 | 90 |
| VII-006 | 1280 | 90 |
| VII-010 | 1280 | 100 |
| VII-009 | 1280 | 100 |

For example, compound nos. 11-005, 11-007, II-011 and 11-013 in tables 1 to 12, at an application rate of 1280 g/ha, each show 90-100% efficacy against Alopecurus myrosoroides, Digitaria sanguinalis, Echinochloa crus-galli, Lolium rigidu and Setaria viridis.

The compounds of the invention are therefore suitable for control of unwanted plant growth by the pre-emergence method.

2. Post-emergence herbicidal effect and crop plant compatibility

Seeds of monocotyledonous and dicotyledonous weed and crop plants are laid out in sandy loam soil in wood-fibre pots, covered with soil and cultivated in a greenhouse under good growth conditions. 2 to 3 weeks after sowing, the test plants are treated at the one-leaf stage. The compounds of the invention, formulated in the form of wettable powders (WP) or as emulsion concentrates (EC), are then sprayed onto the green parts of the plants as aqueous suspension or emulsion at a water application rate equating to 600 to 800 l/ha with addition of 0.2% wetting agent. After the test plants have been left to stand in the greenhouse under optimal growth conditions for about 3 weeks, the action of the preparations is assessed visually in comparison to untreated controls (herbicidal action in percent (%): 100% activity=the plants have died, 0% activity=like control plants).

As shown by the results from Tables 13 to 24, compounds of the invention have good herbicidal post-emergence efficacy against a broad spectrum of weed grasses and broad-leaved weeds.

TABLE 13
Post-emergence action against ALOMY

| Example | Dosage | ALOMY |
|---|---|---|
| number | [g/ha] | [%] |
| I-002 | 1280 | 100 |
| I-005 | 1280 | 100 |
| II-001 | 1280 | 100 |
| I-004 | 1280 | 100 |
| I-006 | 1280 | 100 |
| I-010 | 1280 | 100 |
| I-001 | 1280 | 100 |
| II-005 | 1280 | 100 |
| I-012 | 1280 | 100 |
| II-007 | 1280 | 100 |
| II-011 | 1280 | 100 |
| II-008 | 1280 | 100 |
| I-013 | 1280 | 100 |
| I-013 | 1280 | 100 |
| I-015 | 1280 | 100 |
| I-022 | 1280 | 100 |
| VI-001 | 1280 | 90 |
| I-016 | 1280 | 100 |
| I-020 | 1280 | 100 |
| I-019 | 1280 | 100 |
| I-024 | 1280 | 100 |
| II-012 | 1280 | 90 |
| II-016 | 1280 | 100 |
| I-027 | 1280 | 100 |
| I-014 | 1280 | 100 |
| VII-001 | 1280 | 100 |
| VII-003 | 1280 | 100 |
| VII-005 | 1280 | 100 |
| II-017 | 1280 | 90 |
| VII-011 | 1280 | 90 |
| VII-007 | 1280 | 90 |
| VII-006 | 1280 | 90 |
| VI-002 | 1280 | 90 |
| VII-010 | 1280 | 90 |
| VII-009 | 1280 | 90 |

TABLE 14
Post-emergence action against DIGSA

| Example | Dosage | DIGSA |
|---|---|---|
| number | [g/ha] | [%] |
| I-002 | 1280 | 100 |
| I-005 | 1280 | 100 |
| II-001 | 1280 | 100 |
| I-006 | 1280 | 100 |
| I-010 | 1280 | 90 |
| I-001 | 1280 | 100 |
| II-005 | 1280 | 100 |
| I-012 | 1280 | 100 |
| II-007 | 1280 | 100 |
| II-011 | 1280 | 100 |
| II-008 | 1280 | 100 |
| I-013 | 1280 | 100 |
| I-013 | 1280 | 100 |
| I-022 | 1280 | 90 |
| I-018 | 1280 | 90 |
| I-016 | 1280 | 90 |
| I-020 | 1280 | 90 |
| I-019 | 1280 | 100 |
| I-024 | 1280 | 100 |
| II-016 | 1280 | 100 |
| I-027 | 1280 | 100 |
| I-014 | 1280 | 100 |
| VII-001 | 1280 | 100 |

TABLE 15
Post-emergence action against ECHCG

| Example | Dosage | ECHCG |
|---|---|---|
| number | [g/ha] | [%] |
| II-001 | 1280 | 100 |
| I-010 | 1280 | 100 |
| I-001 | 1280 | 100 |
| II-005 | 1280 | 100 |
| I-012 | 1280 | 100 |
| II-007 | 1280 | 100 |
| II-011 | 1280 | 100 |
| II-008 | 1280 | 100 |
| II-013 | 1280 | 100 |
| I-015 | 1280 | 100 |
| I-022 | 1280 | 100 |
| I-018 | 1280 | 90 |
| I-016 | 1280 | 90 |
| I-020 | 1280 | 90 |
| I-024 | 1280 | 90 |
| II-012 | 1280 | 90 |
| II-016 | 1280 | 100 |
| I-014 | 1280 | 90 |
| VII-001 | 1280 | 100 |
| VII-005 | 1280 | 90 |
| III-005 | 1280 | 90 |
| II-017 | 1280 | 90 |
| VII-007 | 1280 | 90 |
| VI-002 | 1280 | 90 |
| VII-010 | 1280 | 90 |
| VII-009 | 1280 | 90 |

TABLE 16
Post-emergence action against LOLRI

| Example | Dosage | LOLRI |
|---|---|---|
| number | [g/ha] | [%] |
| II-005 | 1280 | 90 |
| I-014 | 1280 | 90 |
| VII-010 | 1280 | 90 |

TABLE 17
Post-emergence action against POAAN

| Example | Dosage | POAAN |
|---|---|---|
| number | [g/ha] | [%] |
| I-009 | 1280 | 100 |
| II-001 | 1280 | 100 |
| I-004 | 1280 | 100 |
| I-006 | 1280 | 100 |
| I-010 | 1280 | 100 |
| III-002 | 1280 | 100 |
| I-001 | 1280 | 100 |
| II-005 | 1280 | 100 |
| I-012 | 1280 | 100 |
| II-007 | 1280 | 100 |
| II-011 | 1280 | 100 |
| II-008 | 1280 | 100 |
| I-013 | 1280 | 100 |
| II-013 | 1280 | 100 |
| I-015 | 1280 | 100 |
| I-022 | 1280 | 100 |
| I-018 | 1280 | 100 |
| VI-001 | 1280 | 100 |
| I-016 | 1280 | 90 |
| I-020 | 1280 | 90 |
| I-019 | 1280 | 100 |
| I-024 | 1280 | 100 |
| II-012 | 1280 | 100 |
| II-015 | 1280 | 90 |
| II-016 | 1280 | 100 |
| I-027 | 1280 | 90 |
| I-014 | 1280 | 100 |
| VII-001 | 1280 | 100 |
| VII-002 | 1280 | 100 |
| VII-003 | 1280 | 100 |
| VII-005 | 1280 | 100 |
| II-017 | 1280 | 100 |
| VII-007 | 1280 | 90 |
| VII-006 | 1280 | 100 |
| VII-010 | 1280 | 100 |
| VII-009 | 1280 | 100 |

TABLE 18
Post-emergence action against SETVI

| Example | Dosage | SETVI |
|---|---|---|
| number | [g/ha] | [%] |
| I-002 | 1280 | 100 |
| II-001 | 1280 | 90 |
| I-001 | 1280 | 90 |
| II-005 | 1280 | 100 |
| I-012 | 1280 | 90 |
| II-007 | 1280 | 90 |
| II-011 | 1280 | 100 |
| II-008 | 1280 | 90 |
| II-013 | 1280 | 90 |
| I-018 | 1280 | 100 |
| I-020 | 1280 | 90 |
| I-019 | 1280 | 90 |
| I-024 | 1280 | 100 |
| II-012 | 1280 | 90 |
| I-014 | 1280 | 90 |
| VII-001 | 1280 | 90 |
| VII-002 | 1280 | 90 |
| VII-005 | 1280 | 100 |
| II-017 | 1280 | 90 |
| VII-007 | 1280 | 90 |
| VII-006 | 1280 | 90 |
| VII-010 | 1280 | 90 |
| VII-009 | 1280 | 90 |

TABLE 19
Post-emergence action against ABUTH

| Example | Dosage | ABUTH |
|---|---|---|
| number | [g/ha] | [%] |
| I-002 | 1280 | 90 |
| I-009 | 1280 | 90 |
| II-001 | 1280 | 90 |
| I-010 | 1280 | 90 |
| I-001 | 1280 | 90 |
| II-005 | 1280 | 90 |
| II-007 | 1280 | 90 |
| II-011 | 1280 | 90 |
| II-008 | 1280 | 90 |
| I-013 | 1280 | 90 |
| II-013 | 1280 | 100 |
| I-015 | 1280 | 90 |
| I-022 | 1280 | 90 |
| I-018 | 1280 | 90 |
| VI-001 | 1280 | 90 |
| I-019 | 1280 | 100 |
| I-024 | 1280 | 90 |
| II-015 | 1280 | 90 |
| I-014 | 1280 | 90 |
| VII-001 | 1280 | 90 |
| VII-005 | 1280 | 100 |
| III-005 | 1280 | 90 |
| VII-008 | 1280 | 90 |
| VII-007 | 1280 | 90 |
| VII-010 | 1280 | 90 |
| VII-009 | 1280 | 90 |

TABLE 20
Post-emergence action against AMARE

| Example | Dosage | AMARE |
|---|---|---|
| number | [g/ha] | [%] |
| I-001 | 1280 | 90 |
| I-002 | 1280 | 90 |
| I-004 | 1280 | 90 |
| I-005 | 1280 | 90 |
| I-006 | 1280 | 100 |
| I-009 | 1280 | 100 |
| I-010 | 1280 | 90 |
| I-012 | 1280 | 90 |
| I-013 | 1280 | 100 |
| II-001 | 1280 | 100 |
| II-005 | 1280 | 90 |
| II-007 | 1280 | 100 |
| II-008 | 1280 | 90 |
| II-011 | 1280 | 90 |
| II-013 | 1280 | 90 |
| IV-001 | 1280 | 90 |
| V-001 | 1280 | 100 |
| VI-001 | 1280 | 90 |

TABLE 21
Post-emergence action against KCHSC

| Example | Dosage | KCHSC |
|---|---|---|
| number | [g/ha] | [%] |
| I-009 | 1280 | 100 |
| I-006 | 1280 | 90 |
| I-010 | 1280 | 90 |
| I-001 | 1280 | 90 |
| II-005 | 1280 | 90 |
| I-012 | 1280 | 90 |
| II-011 | 1280 | 100 |
| I-013 | 1280 | 90 |
| II-013 | 1280 | 90 |
| I-015 | 1280 | 90 |
| I-022 | 1280 | 90 |
| I-016 | 1280 | 90 |
| I-020 | 1280 | 90 |
| I-019 | 1280 | 100 |
| I-024 | 1280 | 90 |
| I-027 | 1280 | 90 |
| I-014 | 1280 | 100 |
| VII-001 | 1280 | 90 |
| VII-005 | 1280 | 100 |
| III-005 | 1280 | 90 |
| VII-011 | 1280 | 90 |
| VII-008 | 1280 | 90 |
| VII-007 | 1280 | 90 |
| VII-006 | 1280 | 90 |
| VII-010 | 1280 | 90 |
| VII-009 | 1280 | 90 |

TABLE 22
Post-emergence action against MATIN

| Example | Dosage | MATIN |
|---|---|---|
| number | [g/ha] | [%] |
| I-002 | 1280 | 100 |
| I-005 | 1280 | 100 |
| I-009 | 1280 | 100 |
| II-001 | 1280 | 100 |
| I-004 | 1280 | 100 |
| I-006 | 1280 | 100 |
| I-010 | 1280 | 100 |
| III-002 | 1280 | 90 |
| I-001 | 1280 | 100 |
| II-005 | 1280 | 100 |
| I-012 | 1280 | 90 |
| II-007 | 1280 | 100 |
| II-011 | 1280 | 90 |
| II-008 | 1280 | 100 |
| I-013 | 1280 | 100 |
| II-013 | 1280 | 100 |
| I-022 | 1280 | 100 |
| I-018 | 1280 | 90 |
| VI-001 | 1280 | 100 |
| I-016 | 1280 | 90 |
| I-020 | 1280 | 90 |
| I-019 | 1280 | 100 |
| I-024 | 1280 | 100 |
| II-012 | 1280 | 90 |
| II-015 | 1280 | 90 |
| II-016 | 1280 | 100 |
| I-014 | 1280 | 100 |
| VII-001 | 1280 | 90 |
| VII-002 | 1280 | 90 |
| VII-003 | 1280 | 90 |
| VII-005 | 1280 | 100 |
| III-005 | 1280 | 90 |
| II-017 | 1280 | 90 |
| VII-007 | 1280 | 90 |
| VII-006 | 1280 | 90 |
| VI-002 | 1280 | 90 |
| VII-010 | 1280 | 90 |
| VII-009 | 1280 | 90 |

TABLE 23
Post-emergence action against STEME

| Example | Dosage | STEME |
|---|---|---|
| number | [g/ha] | [%] |
| I-002 | 1280 | 100 |
| I-005 | 1280 | 100 |
| I-009 | 1280 | 100 |
| II-001 | 1280 | 90 |
| I-004 | 1280 | 90 |
| I-006 | 1280 | 100 |
| I-010 | 1280 | 90 |
| III-002 | 1280 | 90 |
| I-001 | 1280 | 100 |
| II-005 | 1280 | 100 |
| I-012 | 1280 | 100 |
| II-007 | 1280 | 100 |
| II-011 | 1280 | 100 |
| II-008 | 1280 | 100 |
| I-013 | 1280 | 100 |
| II-013 | 1280 | 100 |
| I-022 | 1280 | 90 |
| I-016 | 1280 | 90 |
| I-020 | 1280 | 90 |
| I-019 | 1280 | 90 |
| II-016 | 1280 | 100 |
| I-027 | 1280 | 90 |
| I-014 | 1280 | 100 |
| VII-001 | 1280 | 90 |
| VII-002 | 1280 | 90 |
| VII-003 | 1280 | 90 |
| VII-005 | 1280 | 90 |
| VII-007 | 1280 | 90 |
| VII-009 | 1280 | 100 |

TABLE 24
Post-emergence action against VERPE

| Example | Dosage | VERPE |
|---|---|---|
| number | [g/ha] | [%] |
| I-002 | 1280 | 90 |
| I-005 | 1280 | 90 |
| V-001 | 1280 | 90 |
| IV-001 | 1280 | 90 |
| I-009 | 1280 | 90 |
| II-001 | 1280 | 90 |
| I-004 | 1280 | 90 |
| I-006 | 1280 | 90 |
| I-010 | 1280 | 90 |
| III-002 | 1280 | 100 |
| I-001 | 1280 | 100 |
| II-005 | 1280 | 100 |
| I-012 | 1280 | 100 |
| II-007 | 1280 | 100 |
| II-011 | 1280 | 90 |
| II-008 | 1280 | 100 |
| I-013 | 1280 | 100 |
| II-013 | 1280 | 100 |
| I-015 | 1280 | 100 |
| I-022 | 1280 | 100 |
| I-018 | 1280 | 100 |
| VI-001 | 1280 | 90 |
| I-016 | 1280 | 90 |
| I-020 | 1280 | 90 |
| I-019 | 1280 | 100 |
| I-023 | 1280 | 90 |
| I-024 | 1280 | 100 |
| II-012 | 1280 | 90 |
| II-015 | 1280 | 90 |
| II-016 | 1280 | 100 |
| I-027 | 1280 | 100 |
| I-026 | 1280 | 90 |
| I-014 | 1280 | 90 |
| VII-001 | 1280 | 90 |
| VII-002 | 1280 | 90 |
| VII-003 | 1280 | 90 |
| VII-005 | 1280 | 100 |
| III-005 | 1280 | 90 |
| II-017 | 1280 | 90 |
| VII-011 | 1280 | 90 |
| VII-008 | 1280 | 90 |
| VII-007 | 1280 | 90 |
| VII-006 | 1280 | 90 |
| VI-002 | 1280 | 90 |
| VII-010 | 1280 | 90 |
| VII-009 | 1280 | 90 |

For example, all inventive compounds II-001, 11-005, 11-007 and 11-013 in tables 13 and 24, at an application rate of 1280 g/ha, each show 90-100% efficacy against Alopecurus myosuroides, Digitaria sanguinalis, Setaria viridis and Echinochloa crus-galli, and are therefore suitable for control of unwanted plant growth by the post-emergence method.

1. [(1,4,5-Trisubstituted 1H-pyrazol-3-yl)sulfanyl]acetic acid derivatives of the general formula (I) or an agrochemically acceptable salt thereof, 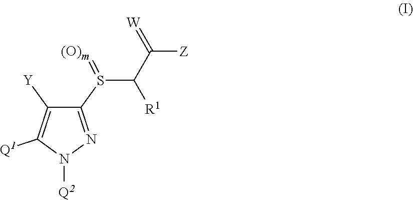
in which
Q¹ is phenyl and hetaryl,
where the phenyl and the hetaryl are unsubstituted or each independently substituted by m radicals selected from the group consisting of hydrogen, halogen, cyano, isocyano, nitro, hydroxy, (C₁-C₆)-alkyl, (C₁-C₆)-haloalkyl, (C₃-C₆)-cycloalkyl, (C₃-C₆)-halocycloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, (C₁-C₄)-haloalkyl-S(O)ₙ, CHO, (C₁-C₄)-alkyloxycarbonyl and NH₂ (amino),
Q² is phenyl,
which is unsubstituted or in each case independently substituted by m radicals selected from the group consisting of hydrogen, halogen, cyano, isocyano, NO₂, (C₁-C₆)-alkyl, (C₁-C₆)-haloalkyl, (C₁-C₆)-haloalkoxy, (C₃-C₆)-cycloalkyl, (C₃-C₆)-halocycloalkyl, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, (C₁-C₄)-haloalkyl-S(O)ₙ, CHO, (C₁-C₄)-alkyloxycarbonyl and NH₂ (amino),
Z is the groups 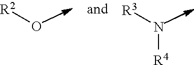
Y is halogen, cyano, isocyano, NO₂, NH₂ (amino), (C₁-C₆)-alkyl, (C₁-C₆)-haloalkyl, (C₁-C₆)-cyanoalkyl, (C₁-C₆)-hydroxyalkyl, (C₁-C₆)-alkoxy-(C₁-C₆)-alkyl, (C₃-C₆)-cycloalkyl, (C₁-C₄-alkyloxycarbonyl, (C₁-C₆)-alkylcarbonyl, CHO, (C₃-C₆)-halocycloalkyl, (C₁-C₆)-alkoxy, (C₁-C₆)-haloalkoxy, (C₁-C₄)-alkyl-S(O)ₙ, (C₁-C₄)-haloalkyl-S(O)ₙ, (C₂-C₆)-alkynyl, (C₂-C₆)-haloalkynyl, (C₂-C₆)-alkenyl and (C₂-C₆)-haloalkenyl,
W is oxygen or sulfur,
R¹ is hydrogen, cyano, (C₁-C₆)-alkyl, (C₁-C₆)-haloalkyl, (C₁-C₆)-alkoxy, (C₁-C₆)-alkoxy-(C₂-C₆)-alkyl, (C₃-C₆)-cycloalkyl, (C₃-C₆)-halocycloalkyl, (C₂-C₆)-alkynyl, (C₂-C₆)-haloalkynyl, (C₂-C₆)-alkenyl and (C₂-C₆)-haloalkenyl,
R² is hydrogen, (C₁-C₁₀)-alkyl and (C₃-C₁₀)-cycloalkyl,
where the alkyl and cycloalkyl are unsubstituted or each independently substituted by m radicals selected from the group consisting of halogen, cyano, isocyano, nitro, NH₂ (amino), (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, CHO, (C₁-C₄)-alkyloxycarbonyl, heterocyclyl-(C₁-C₄)-alkyl, heteroaryl-(C₁-C₄)-alkyl and aryl-(C₁-C₄)-alkyl, where the aryl, heterocyclyl and heteroaryl are unsubstituted or each independently substituted by m radicals selected from the group consisting of halogen, (C₁-C₆)-alkyl, (C₁-C₆)-haloalkyl,
R³ is hydrogen and (C₁-C₁₂)-alkyl,
R⁴ is hydrogen, cyano, nitro, (C₁-C₁₂)-alkyl, (C₁-C₁₀)-haloalkyl, (C₂-C₁₀)-alkenyl, (C₃-C₁₀)-alkynyl, (C₁-C₁₀)-alkoxy-(C₁-C₁₀) -alkyl, (C₁-C₁₀)-haloalkoxy-(C₁-C₁₀)-alkyl, (C₁-C₄)-alkyl-S(O)ₙ—(C₁-C₄)-alkyl, (C₁-C₄)-haloalkyl-S(O)ₙ—(C₁-C₄)-alkyl, (C₃-C₁₀)-cycloalkyl, (C₃-C₁₀)-halocycloalkyl, hydroxy-(C₁-C₁₀)-alkylcarbonyl, amino-(C₁-C₁₀)-alkyl, (C₁-C₁₀)-alkoxycarbonyl-(C₁-C₁₀)-alkyl, (C₁-C₁₀)-cyanoalkyl, S(O)ₙR⁵, OR⁵, SO₂NR⁶R⁷, CO₂R⁸, COR⁶, NR⁶R⁸, NR⁶COR⁸, NR⁶CO₂R⁸, NR⁶SO₂R⁸, aryl, heteroaryl and heterocyclyl,
which is unsubstituted or each independently substituted by m radicals selected from the group consisting of hydrogen, halogen, cyano, nitro, OR⁵, S(O)ₙR⁵, SO₂NR⁶R⁷, CO₂R⁸, CONR⁶R⁸, COR^{E}, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;
Or
R³ and R⁴ together with the nitrogen atom to which they are bonded form a fully saturated or partly saturated 3- to 10-membered monocyclic or bicyclic ring optionally interrupted by heteroatoms and optionally having further substitution,
R⁵ is hydrogen, (C₁-C₈)-alkyl, (C₃-C₆)-cycloalkyl, (C₁-C₈)-haloalkyl and aryl,
R⁶ is hydrogen and R⁵,
R⁷ is hydrogen, (C₁-C₆)-alkyl, (C₃-C₆)-cycloalkyl, (C₃-C₄)-alkenyl, (C₃-C₄)-alkynyl and (C₁-C₁₀)-alkylcarbonyl-(C₁-C₆)-alkyl,
R⁸ is hydrogen, (C₁-C₆)-alkyl, (C₃-C₆)-cycloalkyl, (C₃-C₄)-alkenyl, and (C₃-C₄)-alkynyl,
m is 0, 1 or 2,
and
n is 0, 1 or 2,
excluding the compounds ethyl {[4-nitro-1-phenyl-5-(1H-tetrazol-5-yl)-1H-pyrazol-3-yl]sulfanyl}acetate CAS [1360702-86-8] and methyl {[5-(5-methyl-1,3,4-oxadiazol-2-yl)-4-nitro-1-phenyl-1H-pyrazol-3-yl]sulfanyl}acetate CAS [1360690-60-3].

2. Compounds of the formula (I) according to claim 1 or an agrochemically acceptable salt thereof, in which
Q¹ is the groups Q¹-1.1 to Q¹-6.5 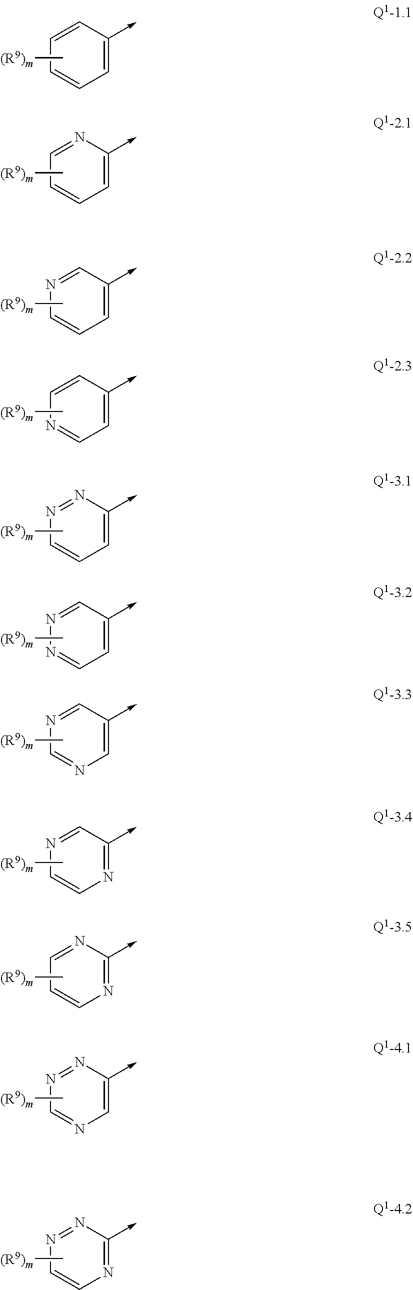 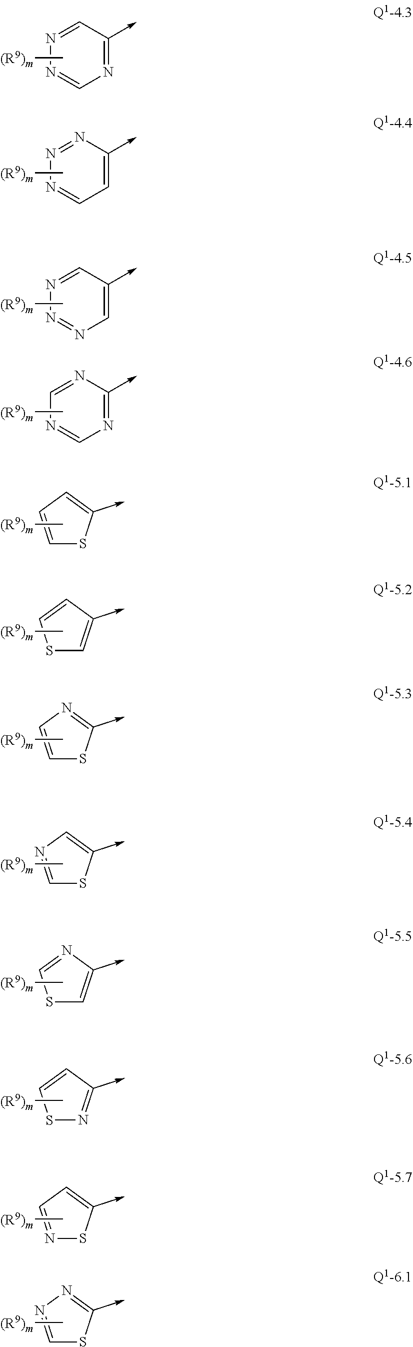 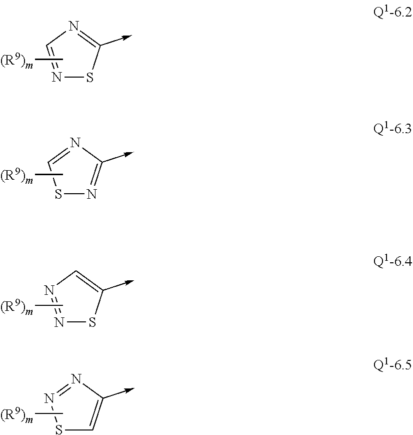
Q² is phenyl,
which is unsubstituted or in each case independently substituted by m radicals selected from the group consisting of hydrogen, halogen, cyano, isocyano, NO₂, (C₁-C₆)-alkyl, (C₁-C₆)-haloalkyl, (C₁-C₆)-haloalkoxy, (C₃-C₆)-cycloalkyl, (C₃-C₆)-halocycloalkyl, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, (C₁-C₄)-haloalkyl-S(O)ₙ, CHO, (C₁-C₄)-alkyloxycarbonyl and NH₂ (amino),
Z is the groups 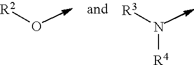
Y is halogen, cyano, isocyano, NO₂, NH₂ (amino), (C₁-C₆)-alkyl, (C₁-C₆)-haloalkyl, (C₁-C₆)-cyanoalkyl, (C₁-C₆)-hydroxyalkyl, (C₁-C₆)-alkoxy-(C₁-C₆)-alkyl, (C₃-C₆)-cycloalkyl, (C₁-C₄)-alkyloxycarbonyl, (C₁-C₆)-alkylcarbonyl, CHO, (C₃-C₆)-halocycloalkyl, (C₁-C₆)-alkoxy, (C₁-C₆)-haloalkoxy, (C₁-C₄)-alkyl-S(O)ₙ, (C₁-C₄)-haloalkyl-S(O)ₙ, (C₂-C₆)-alkynyl, (C₂-C₆)-haloalkynyl, (C₂-C₆)-alkenyl and (C₂-C₆)-haloalkenyl,
W is oxygen or sulfur,
R¹ is hydrogen, cyano, (C₁-C₆)-alkyl, (C₁-C₆)-haloalkyl, (C₁-C₆)-alkoxy, (C₁-C₆)-alkoxy-(C₁-C₆)-alkyl, (C₃-C₆)-cycloalkyl, (C₃-C₆)-halocycloalkyl, (C₂-C₆)-alkynyl, (C₂-C₆)-haloalkynyl, (C₂-C₆)-alkenyl and (C₂-C₆)-haloalkenyl,
R² is hydrogen, (C₁-C₁₀)-alkyl and (C₃-C₁₀)-cycloalkyl,
where the alkyl and cycloalkyl are unsubstituted or each independently substituted by m radicals selected from the group consisting of halogen, cyano, isocyano, nitro, NH₂ (amino), (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, CHO, (C₁-C₄)-alkyloxycarbonyl, heterocyclyl-(C₁-C₆)-alkyl, heteroaryl-(C₁-C₄)-alkyl and aryl-(C₁-C₄)-alkyl, where the aryl, heterocyclyl and heteroaryl are unsubstituted or each independently substituted by m radicals selected from the group consisting of halogen, (C₁-C₆)-alkyl and (C₁-C₆)-haloalkyl,
R³ is hydrogen and (C₁-C₁₀)-alkyl,
R⁴ is hydrogen, cyano, nitro, (C₁-C₁₀)-alkyl, (C₁-C₁₀)-haloalkyl, (C₂-C₁₀)-alkenyl, (C₃-C₁₀)-alkyl, (C₁-C₁₀)-alkoxy-(C₁-C₁₀)-alkyl, (C₁-C₁₀)-haloalkoxy-(C₁-C₁₀)-alkyl, (C₁-C₄)-alkyl-S(O)ₙ—(C₁-C₄)-alkyl, (C₁-C₄)-haloalkyl-S(O)ₙ—(C₁-C₄)-alkyl, (C₃-C₁₀)-cycloalkyl, (C₃-C₁₀)-halocycloalkyl, hydroxy-(C₁-C₁₀)-alkylcarbonyl, amino-(C₁-C₁₀)-alkyl, (C₁-C₁₀)-alkoxycarbonyl-(C₁-C₁₀)-alkyl, (C₁-C₁₀)-cyanoalkyl, S(O)ₙR⁵, OR⁵, SO₂NR⁶R⁷, CO₂R⁸, COR⁸, NR⁶R⁸, NR⁶COR⁸, NR⁶CO₂R⁸, NR⁶SO₂R⁸; aryl, heteroaryl and heterocyclyl,
which are unsubstituted or each independently substituted by m radicals selected from the group consisting of hydrogen, halogen, cyano, nitro, OR⁵, S(O)ₙR⁵, SO₂NR⁶R⁷, CO₂R⁸, CONR⁶R⁸, COR^{E}, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;
or R³ and R⁴ together with the nitrogen atom to which they are bonded form a fully saturated or partly saturated 3- to 10-membered monocyclic or bicyclic ring optionally interrupted by heteroatoms and optionally having further substitution,
R⁵ is hydrogen, (C₁-C₈)-alkyl, (C₃-C₆)-cycloalkyl, (C₁-C₈)-haloalkyl and aryl,
R⁶ is hydrogen and R⁵,
R⁷ is hydrogen, (C₁-C₆)-alkyl, (C₃-C₆)-cycloalkyl, (C₃-C₄)-alkenyl, (C₃-C₄)-alkynyl and (C₁-C₆)-alkylcarbonyl-(C₁-C₄)-alkyl,
R⁸ is hydrogen, (C₁-C₆)-alkyl, (C₃-C₆)-cycloalkyl, (C₃-C₄)-alkenyl and (C₃-C₄)-alkynyl,
R⁹ is hydrogen, halogen, cyano, isocyano, nitro, hydroxy, (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, CHO, (C₁-C₄)-alkyloxycarbonyl and NH₂ (amino),
m is 0,1, and 2,
n is 0, 1 and 2.

3. Compounds of the formula (I) according to claim 1 or an agrochemically acceptable salt thereof, in which
Q¹ is the groups Q¹-1.1 to Q¹-6.3, 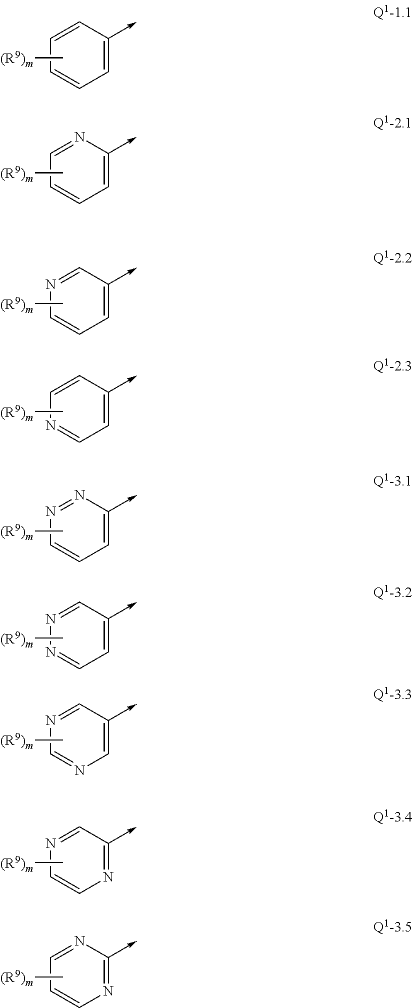 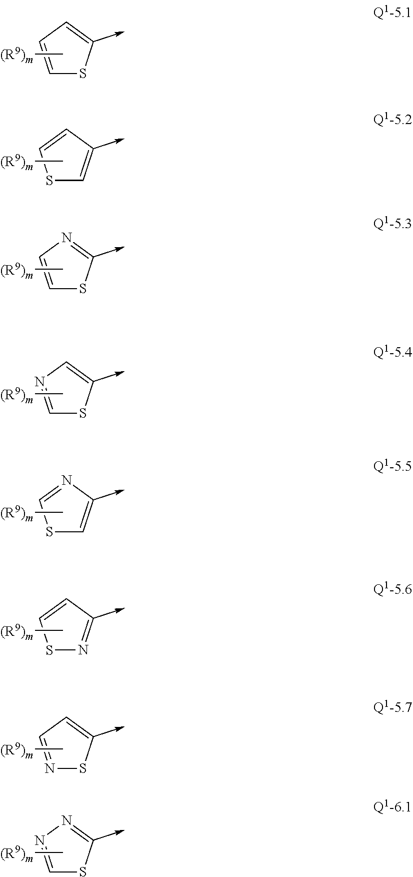 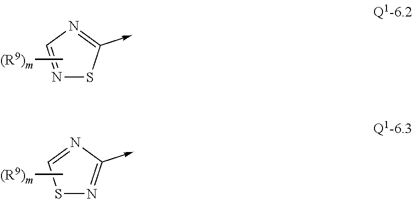
Q² is phenyl,
which is unsubstituted or in each case independently substituted by m radicals selected from the group consisting of hydrogen, halogen, cyano, (C₁-C₆)-alkyl, (C₁-C₆)-haloalkyl, (C₁-C₆)-haloalkoxy, (C₃-C₆)-cycloalkyl, (C₁-C₆)-alkoxy, (C₁-C₄)-alkyl-S(O)ₙ and (C₁-C₄)-haloalkyl-S(O)ₙ,
Z is the groups 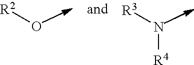
Y is halogen, cyano, isocyano, NO₂, (C₁-C₆)-alkyl, (C₁-C₆)-haloalkyl, (C₁-C₆)-alkoxy-(C₁-C₆)-alkyl, (C₁-C₄)-alkyloxycarbonyl, (C₁-C₆)-alkylcarbonyl, (C₁-C₆)-alkoxy, (C₁-C₆)-haloalkoxy, (C₁-C₄)-alkyl-S(O)ₙ, (C₁-C₄)-haloalkyl-S(O)ₙ, (C₂-C₆)-alkynyl, (C₂-C₆)-haloalkynyl, (C₂-C₆)-alkenyl and (C₂-C₆)-haloalkenyl,
W is oxygen,
R¹ is hydrogen, cyano, (C₁-C₆)-alkyl, (C₁-C₆)-haloalkyl, (C₁-C₆)-alkoxy, (C₁-C₄)-alkoxy-(C₁-C₄)-alkyl, (C₃-C₆)-cycloalkyl, (C₂-C₆)-alkynyl, (C₂-C₆)-haloalkynyl, (C₂-C₆)-alkenyl and (C₂-C₆)-haloalkenyl,
R² is hydrogen, (C₁-C₆)-alkyl and (C₃-C₆)-cycloalkyl,
where the alkyl and cycloalkyl are unsubstituted or each independently substituted by m radicals selected from the group consisting of halogen, cyano, nitro, NH₂ (amino), (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, CHO, (C₁-C₄)-alkyloxycarbonyl, heterocyclyl-(C₁-C₄)-alkyl, heteroaryl-(C₁-C₄)-alkyl and aryl-(C₁-C₄)-alkyl, where the aryl, heterocyclyl and heteroaryl are unsubstituted or each independently substituted by m radicals selected from the group consisting of halogen, (C₁-C₆)-alkyl and (C₁-C₆)-haloalkyl,
R³ is hydrogen and (C₁-C₆)-alkyl,
R⁴ is hydrogen, cyano, nitro, (C₁-C₆)-alkyl, (C₁-C₆)-haloalkyl, (C₂-C₆)-alkenyl, (C₃-C₆)-alkynyl, (C₁-C₆)-alkoxy-(C₁-C₆)-alkyl, (C₁-C₆)-haloalkoxy-(C₁-C₆)-alkyl, (C₁-C₄)-alkyl-S(O)ₙ—(C₁-C₄)-alkyl and (C₁-C₄)-haloalkyl-S(O)ₙ—(C₁-C₄)-alkyl, (C₃-C₆)-cycloalkyl, (C₃-C₆)-halocycloalkyl, hydroxy-(C₁-C₆)-alkylcarbonyl, amino-(C₁-C₆)-alkyl, (C₁-C₆)-alkoxycarbonyl-(C₁-C₆)-alkyl, S(O)₁₁R⁵, OR⁵, SO₂NR⁶R⁷, CO₂R⁸, COR⁶, NR⁶R⁸, NR⁶COR⁸, NR⁶CO₂R⁸, NR⁶SO₂R⁸; aryl, heteroaryl and heterocyclyl,
which are unsubstituted or each independently substituted by m radicals selected from the group consisting of hydrogen, halogen, cyano, nitro, OR⁵, S(O)ₙR⁵, SO₂NR⁶R⁷, CO₂R⁸, CONR⁶R⁸, COR^{E}, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;
or R³ and R⁴ together with the nitrogen atom to which they are bonded form a fully saturated or partly saturated 3- to 10-membered monocyclic or bicyclic ring optionally interrupted by heteroatoms and optionally having further substitution,
R⁵ is hydrogen, (C₁-C₈)-alkyl, (C₃-C₆)-cycloalkyl, (C₁-C₈)-haloalkyl and aryl,
R⁶ is hydrogen and R⁵,
R⁷ is hydrogen, (C₁-C₆)-alkyl, (C₃-C₆)-cycloalkyl, (C₃-C₄)-alkenyl, and (C₃-C₄)-alkynyl,
R⁸ is hydrogen, (C₁-C₆)-alkyl, (C₃-C₆)-cycloalkyl, (C₃-C₄)-alkenyl, (C₃-C₄)-alkynyl and (C₁-C₁₀)-alkylcarbonyl-(C₁-C₆)-alkyl,
R⁹ is hydrogen, halogen, cyano, isocyano, nitro, hydroxy, (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, CHO, (C₁-C₄)-alkyloxycarbonyl and NH₂ (amino),
m is 0, 1 and 2,
n is 0, 1 and 2.

4. Compounds of the formula (I) according to claim 1 or an agrochemically acceptable salt thereof, in which
Q¹ is the groups Q¹-1.1 to Q¹-6.3, 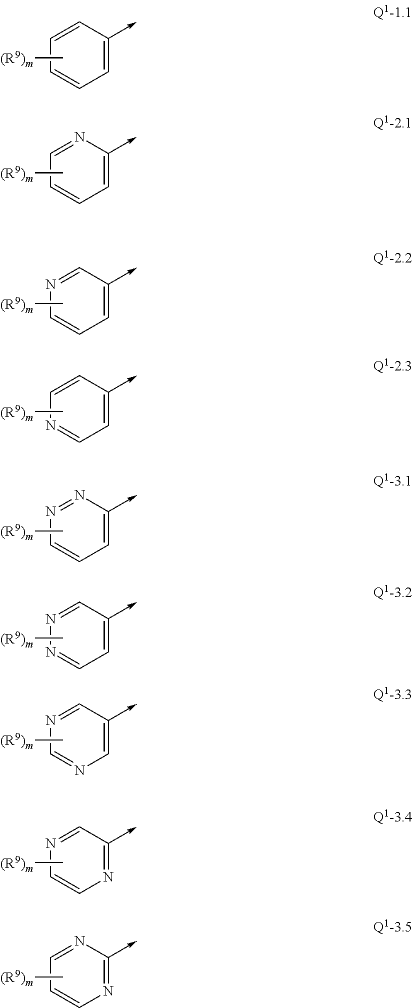 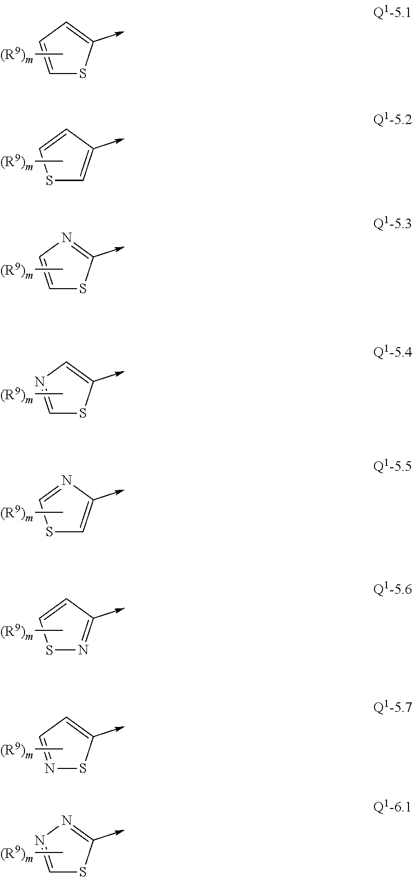 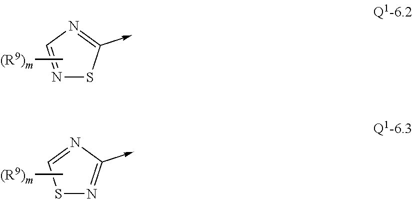
Q² is phenyl,
which is unsubstituted or in each case independently substituted by m radicals selected from the group consisting of hydrogen, fluorine, chlorine and bromine,
Z is the groups 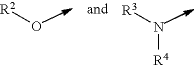
Y is fluorine, chlorine, bromine, cyano, NO₂, (C₁-C₂)-alkyl, (C₁-C₂)-haloalkyl, (C₁-C₂)-alkylcarbonyl, (C₁-C₂)-alkoxy, (C₁-C₂)-haloalkoxy, (C₁-C₂)-alkyl-S(O)ₙ, and (C₁-C₂)-haloalkyl-S(O)ₙ,
W is oxygen,
R¹ is hydrogen, (C₁-C₄)-alkyl, (C₁-C₄)-haloalkyl and (C₁-C₆)-alkoxy,
R² is hydrogen, (C₁-C₆)-alkyl and (C₃-C₆)-cycloalkyl,
where the alkyl and cycloalkyl are unsubstituted or each independently substituted by m radicals selected from the group consisting of halogen, cyano, nitro, NH₂ (amino), (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, CHO, (C₁-C₄)-alkyloxycarbonyl, heterocyclyl-(C₁-C₄)-alkyl, heteroaryl-(C₁-C₄)-alkyl and aryl-(C₁-C₄)-alkyl, where the aryl, heterocyclyl and heteroaryl are unsubstituted or each independently substituted by m radicals selected from the group consisting of halogen, (C₁-C₆)-alkyl and (C₁-C₆)-haloalkyl,
R³ is hydrogen and (C₁-C₆)-alkyl,
R⁴ is hydrogen, cyano, nitro, (C₁-C₆)-alkyl, (C₁-C₆)-haloalkyl, (C₂-C₆)-alkenyl, (C₃-C₆)-alkynyl, (C₁-C₆)-alkoxy-(C₁-C₆)-alkyl, (C₁-C₆)-haloalkoxy-(C₁-C₆)-alkyl, (C₁-C₄)-alkyl-S(O)ₙ—(C₁-C₄)-alkyl and (C₁-C₄)-haloalkyl-S(O)ₙ—(C₁-C₄)-alkyl, (C₃-C₆)-cycloalkyl, (C₃-C₆)-halocycloalkyl, hydroxy-(C₁-C₆)-alkylcarbonyl, amino-(C₁-C₆)-alkyl, (C₁-C₆)-alkoxycarbonyl-(C₁-C₆)-alkyl, S(O)ₙR⁵, OR⁵, SO₂NR⁶R⁷, CO₂R⁸, COR⁶, NR⁶R⁸, NR⁶COR⁸, NR⁶CO₂R⁸, NR⁶SO₂R⁸; aryl, heteroaryl and heterocyclyl,
which are unsubstituted or each independently substituted by m radicals selected from the group consisting of hydrogen, halogen, cyano, nitro, OR⁵, S(O)ₙR⁵, SO₂NR⁶R⁸, CO₂R⁸, CONR⁶R⁸, COR^{E}, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;
or R³ and R⁴ together with the nitrogen atom to which they are bonded form a fully saturated or partly saturated 3- to 10-membered monocyclic or bicyclic ring optionally interrupted by heteroatoms and optionally having further substitution,
R⁵ is hydrogen, (C₁-C₈)-alkyl, (C₃-C₆)-cycloalkyl, (C₁-C₈)-haloalkyl and aryl,
R⁶ is hydrogen and R⁵,
R⁷ is hydrogen, (C₁-C₆)-alkyl, (C₃-C₆)-cycloalkyl, (C₃-C₄)-alkenyl, (C₃-C₄)-alkynyl and (C₁-C₁₀)-alkylcarbonyl-(C₁-C₆)-alkyl,
R⁸ is hydrogen, (C₁-C₆)-alkyl, (C₃-C₆)-cycloalkyl, (C₃-C₄)-alkenyl and (C₃-C₄)-alkynyl,
R⁹ is hydrogen, halogen, cyano, isocyano, nitro, hydroxy, (C₁-C₆)-alkyl, (C₁-C₆)-haloalkyl, (C₁-C₆)-haloalkoxy, (C₂-C₃)-alkenyl, (C₂-C₃)-haloalkenyl, (C₁-C₆)-alkoxy, (C₂-C₃)-alkynyl, (C₂-C₃)-haloalkynyl, (C₁-C₄)-alkyl-S(O)ₙ, CHO, (C₁-C₄)-alkyloxycarbonyl and NH₂ (amino),
m is 0, 1 and 2,
n is 0, 1 and 2.

5. Compounds of the formula (I) according to claim 1 or an agrochemically acceptable salt thereof, in which
Q¹ is the groups Q¹-1.1 to Q¹-5.7 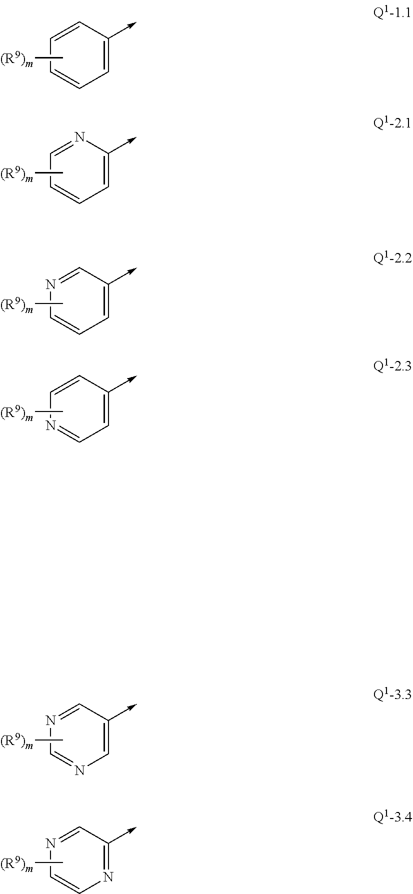 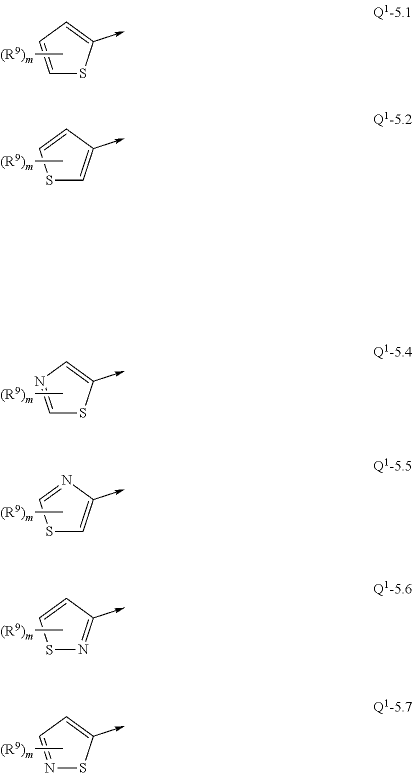
Q² is phenyl,
which is unsubstituted or in each case independently substituted by m radicals selected from the group consisting of hydrogen, fluorine, chlorine and bromine,
Z is the groups 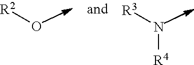
Y is fluorine, chlorine, bromine, cyano, NO₂, methyl, CF₃ and OCF₃,
W is oxygen,
R¹ is hydrogen, methyl and ethyl,
R² is hydrogen, methyl, ethyl, allyl, propargyl and PhCH₂,
R³ is hydrogen,
R⁴ is (C₁-C₆)-alkyl, S(O)₁₁R⁵, OR⁵, SO₂NR⁶R⁷, CO₂R⁸, COR⁶,
which are unsubstituted or in each case independently of one another substituted by m radicals selected from the group consisting of hydrogen, OR⁵, S(O)₁₁R⁵, SO₂NR⁶R⁷, CO₂R⁸, COR^{E}, NR⁶CO₂R⁸,
R⁵ is methyl, ethyl, CF₃, CH₂CF₃,
R⁶ is hydrogen and R⁵,
R⁷ is hydrogen, methyl, ethyl, and ethyl-2-ethanoyl,
R⁸ is methyl and ethyl,
R⁹ is hydrogen, fluorine, chlorine, bromine, cyano, hydroxy, methyl, ethyl, OCH₃, CF₃, and OCF₃,
m is 0, 1 and 2,
n is 0, 1 and 2.

6. Process for preparing compounds of the general formula (Ia) or an agrochemically acceptable salt thereof according to claim 1 by converting compounds of the general formulae (II) and (III) to compounds of the general formula (Ia) 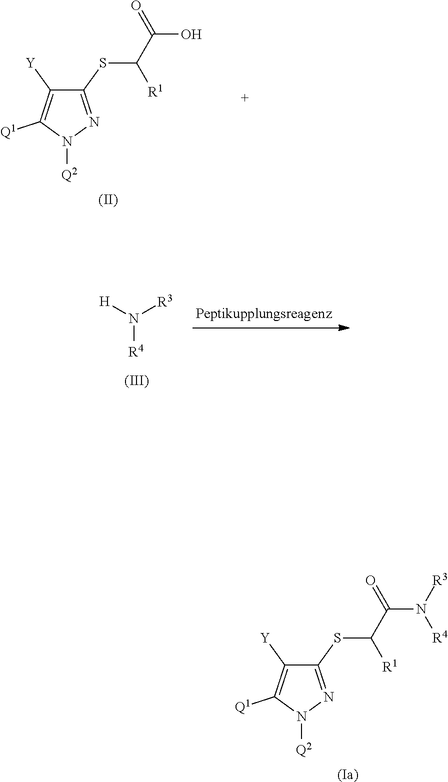
in which Q¹, Q², Y, R¹, R², R³ and R⁴ have the definitions given above.

7. Agrochemical composition comprising a) at least one compound of the formula (I) or an agrochemically acceptable salt thereof as defined in claim 1, and b) auxiliaries and additives customary in crop protection.

8. Agrochemical composition comprising
a) at least one compound of the formula (I) or an agrochemically acceptable salt thereof as defined in claim 1,
b) one or more active agrochemical ingredients other than component a), and optionally
c) auxiliaries and additives customary in crop protection.

9. Method of controlling unwanted plants or for regulating the growth of plants, wherein an effective amount of at least one compound of the formula (I) or an agrochemically acceptable salt thereof, as defined in claim 1, is applied to the plants, the seed or the area in which the plants grow.

10. Use of compounds of the formula (I) or an agrochemically acceptable salt thereof, as defined in claim 1, as herbicides or plant growth regulators.

11. Use according to claim 10, wherein the compounds of the formula (I) or an agrochemically acceptable salt thereof are used for controlling harmful plants or for regulating growth in plant crops.

12. Use according to claim 11, wherein the crop plants are transgenic or nontransgenic crop plants.
